SEQUENCE LISTING (68 sequences)

SEQ ID NO: 1 - RNA, 41 nt, Artificial Sequence
  source: /note=“Description of Artificial Sequence Synthetic oligonucleotide”
ccaguagccagccccguccguuuuagagcuaugcuguuuug

SEQ ID NO: 2 - RNA, 85 nt, Artificial Sequence
  source: /note=“Description of Artificial Sequence Synthetic oligonucleotide”
ggaaccauucaaaacagcauagcaaguuaaaauaaggcuaguccguuaucaacuugaaaaagggcaccgagucggugcuuuuuuu

SEQ ID NO: 3 - RNA, 104 nt, Artificial Sequence
  source: /note=“Description of Artificial Sequence Synthetic polynucleotide”
gcauuucgguaugcauaugaaugaguuuuagagcuagaaauagcaaguuaaaauaaggcuaguccguuaucaacuugaaaaaguggcaccgagucggugcuuuu

SEQ ID NO: 4 - DNA, 23 bp, Artificial Sequence
  source: /note=“Description of Artificial Sequence Synthetic oligonucleotide”
ccatcatatgcataccgaaatgc

SEQ ID NO: 5 - DNA, 23 bp, Artificial Sequence
  source: /note=“Description of Artificial Sequence Synthetic oligonucleotide”
  modified_base (21)..(21): a, c, t, g, unknown or other
gcatttcggtatgcatatgangg

SEQ ID NO: 6 - DNA, 23 bp, Artificial Sequence
  source: /note=“Description of Artificial Sequence Synthetic primer”
gttttagagctagaaatagcaag

SEQ ID NO: 7 - DNA, 22 bp, Artificial Sequence
  source: /note=“Description of Artificial Sequence Synthetic primer”
aaaagcaccgactcggtgccac

SEQ ID NO: 8 - DNA, 19 bp, Artificial Sequence
  source: /note=“Description of Artificial Sequence Synthetic primer”
taatacgactcactatagg

SEQ ID NO: 9 - DNA, 14 bp, Artificial Sequence
  source: /note=“Description of Artificial Sequence Synthetic oligonucleotide”
gttttagagctaga

SEQ ID NO: 10 - DNA, 19 bp, Artificial Sequence
  source: /note=“Description of Artificial Sequence Synthetic oligonucleotide”
catttctcagtcctaaaca

SEQ ID NO: 11 - DNA, 20 bp, Artificial Sequence
  source: /note=“Description of Artificial Sequence Synthetic oligonucleotide”
taatacgactcactataggg

SEQ ID NO: 12 - DNA, 19 bp, Artificial Sequence
  source: /note=“Description of Artificial Sequence Synthetic oligonucleotide”
ccagtagccagccccgtcc

SEQ ID NO: 13 - DNA, 19 bp, Artificial Sequence
  source: /note=“Description of Artificial Sequence Synthetic oligonucleotide”
tcgtgtccctgtacgcgga

SEQ ID NO: 14 - DNA, 40 bp, Artificial Sequence
  source: /note=“Description of Artificial Sequence Synthetic oligonucleotide”
taatacgactcactataggggcatttctcagtcctaaaca

SEQ ID NO: 15 - DNA, 40 bp, Artificial Sequence
  source: /note=“Description of Artificial Sequence Synthetic oligonucleotide”
gctatttctagctctaaaactgtttaggactgagaaatgc

SEQ ID NO: 16 - DNA, 60 bp, Artificial Sequence
  source: /note=“Description of Artificial Sequence Synthetic oligonucleotide”
gttttagagctagaaatagcaagttaaaataaggctagtccgttatcaacttgaaaaagt

SEQ ID NO: 17 - DNA, 60 bp, Artificial Sequence
  source: /note=“Description of Artificial Sequence Synthetic oligonucleotide”
aaaagcaccgactcggtgccactttttcaagttgataacggactagccttattttaactt

SEQ ID NO: 18 - DNA, 22 bp, Artificial Sequence
  source: /note=“Description of Artificial Sequence Synthetic oligonucleotide”
aaaagcaccgactcggtgccac

SEQ ID NO: 19 - DNA, 23 bp, Artificial Sequence
  source: /note=“Description of Artificial Sequence Synthetic oligonucleotide”
gttttagagctagaaatagcaag

SEQ ID NO: 20 - DNA, 22 bp, Artificial Sequence
  source: /note=“Description of Artificial Sequence Synthetic oligonucleotide”
aaaagcaccgactcggtgccac

SEQ ID NO: 21 - DNA, 39 bp, Artificial Sequence
  source: /note=“Description of Artificial Sequence Synthetic oligonucleotide”
gctatttctagctctaaaactgtttaggactgagaaatg

SEQ ID NO: 22 - DNA, 34 bp, Artificial Sequence
  source: /note=“Description of Artificial Sequence Synthetic oligonucleotide”
ttctagctctaaaactgtttaggactgagaaatg

SEQ ID NO: 23 - DNA, 39 bp, Artificial Sequence
  source: /note=“Description of Artificial Sequence Synthetic oligonucleotide”
taatacgactcactataggcatttctcagtcctaaacag

SEQ ID NO: 24 - DNA, 34 bp, Artificial Sequence
  source: /note=“Description of Artificial Sequence Synthetic oligonucleotide”
taatacgactcactataggcatttctcagtccta

SEQ ID NO: 25 - DNA, 19 bp, Artificial Sequence
  source: /note=“Description of Artificial Sequence Synthetic oligonucleotide”
taatacgactcactatagg

SEQ ID NO: 26 - DNA, 22 bp, Artificial Sequence
  source: /note=“Description of Artificial Sequence Synthetic oligonucleotide”
aaaagcaccgactcggtgccac

SEQ ID NO: 27 - DNA, 21 bp, Artificial Sequence
  source: /note=“Description of Artificial Sequence Synthetic oligonucleotide”
aaagcaccgactcggtgccac

SEQ ID NO: 28 - DNA, 20 bp, Artificial Sequence
  source: /note=“Description of Artificial Sequence Synthetic oligonucleotide”
aagcaccgactcggtgccac

SEQ ID NO: 29 - DNA, 19 bp, Artificial Sequence
  source: /note=“Description of Artificial Sequence Synthetic oligonucleotide”
agcaccgactcggtgccac

SEQ ID NO: 30 - DNA, 18 bp, Artificial Sequence
  source: /note=“Description of Artificial Sequence Synthetic oligonucleotide”
gcaccgactcggtgccac

SEQ ID NO: 31 - DNA, 35 bp, Artificial Sequence
  source: /note=“Description of Artificial Sequence Synthetic oligonucleotide”
taatacgactcactatagggccagtagccagcccc

SEQ ID NO: 32 - DNA, 34 bp, Artificial Sequence
  source: /note=“Description of Artificial Sequence Synthetic oligonucleotide”
taatacgactcactataggccagtagccagcccc

SEQ ID NO: 33 - DNA, 33 bp, Artificial Sequence
  source: /note=“Description of Artificial Sequence Synthetic oligonucleotide”
taatacgactcactatagccagtagccagcccc

SEQ ID NO: 34 - DNA, 9823 bp, Artificial Sequence
  source: /note=“Description of Artificial Sequence Synthetic polynucleotide”
  modified_base (7336)..(7355): a, c, t, g, unknown or other
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggagcggccgccaccatgggcaagcccatccctaaccccctgttggggctggacagcaccgctcccaaaaagaaaaggaaggtgggcattcacggcgtgcctgcggccgacaaaaagtacagcatcggccttgatatcggcaccaatagcgtgggctgggccgttatcacagacgaatacaaggtacccagcaagaagttcaaggtgctggggaatacagacaggcactctatcaagaaaaaccttatcggggctctgctgtttgactcaggcgagaccgccgaggccaccaggttgaagaggaccgcaaggcgaaggtacacccggaggaagaacaggatctgctatctgcaggagatcttcagcaacgagatggccaaggtggacgacagcttcttccacaggctggaggagagcttccttgtcgaggaggataagaagcacgaacgacaccccatcttcggcaacatagtcgacgaggtcgcttatcacgagaagtaccccaccatctaccacctgcgaaagaaattggtggatagcaccgataaagccgacttgcgacttatctacttggctctggcgcacatgattaagttcaggggccacttcctgatcgagggcgaccttaaccccgacaacagtgacgtagacaaattgttcatccagcttgtacagacctataaccagctgttcgaggaaaaccctattaacgccagcggggtggatgcgaaggccatacttagcgccaggctgagcaaaagcaggcgcttggagaacctgatagcccagctgcccggtgaaaagaagaacggcctcttcggtaatctgattgccctgagcctgggcctgacccccaacttcaagagcaacttcgacctggcagaagatgccaagctgcagttgagtaaggacacctatgacgacgacttggacaatctgctcgcccaaatcggcgaccagtacgctgacctgttcctcgccgccaagaacctttctgacgcaatcctgcttagcgatatccttagggtgaacacagagatcaccaaggcccccctgagcgccagcatgatcaagaggtacgacgagcaccatcaggacctgacccttctgaaggccctggtgaggcagcaactgcccgagaagtacaaggagatctttttcgaccagagcaagaacggctacgccggctacatcgacggcggagccagccaagaggagttctacaagttcatcaagcccatcctggagaagatggatggcaccgaggagctgctggtgaagctgaacagggaagatttgctccggaagcagaggacctttgacaacggtagcatcccccaccagatccacctgggcgagctgcacgcaatactgaggcgacaggaggatttctaccccttcctcaaggacaatagggagaaaatcgaaaagattctgaccttcaggatcccctactacgtgggccctcttgccaggggcaacagccgattcgcttggatgacaagaaagagcgaggagaccatcaccccctggaacttcgaggaagtggtggacaaaggagcaagcgcgcagtctttcatcgaacggatgaccaatttcgacaaaaacctgcctaacgagaaggtgctgcccaagcacagcctgctttacgagtacttcaccgtgtacaacgagctcaccaaggtgaaatatgtgaccgagggcatgcgaaaacccgctttcctgagcggcgagcagaagaaggccatcgtggacctgctgttcaagaccaacaggaaggtgaccgtgaagcagctgaaggaggactacttcaagaagatcgagtgctttgatagcgtggaaataagcggcgtggaggacaggttcaacgccagcctgggcacctaccacgacttgttgaagataatcaaagacaaggatttcctggataatgaggagaacgaggatatactcgaggacatcgtgctgactttgaccctgtttgaggaccgagagatgattgaagaaaggctcaaaacctacgcccacctgttcgacgacaaagtgatgaaacaactgaagagacgaagatacaccggctggggcagactgtccaggaagctcatcaacggcattagggacaagcagagcggcaagaccatcctggatttcctgaagtccgacggcttcgccaaccgaaacttcatgcagctgattcacgatgacagcttgaccttcaaggaggacatccagaaggcccaggttagcggccagggcgactccctgcacgaacatattgcaaacctggcaggctcccctgcgatcaagaagggcatactgcagaccgttaaggttgtggacgaattggtcaaggtcatgggcaggcacaagcccgaaaacatagttatagagatggccagagagaaccagaccacccaaaagggccagaagaacagccgggagcgcatgaaaaggatcgaggagggtatcaaggaactcggaagccagatcctcaaagagcaccccgtggagaatacccagctccagaacgagaagctgtacctgtactacctgcagaacggcagggacatgtacgttgaccaggagttggacatcaacaggctttcagactatgacgtggatcacatagtgccccagagctttcttaaagacgatagcatcgacaacaaggtcctgacccgctccgacaaaaacaggggcaaaagcgacaacgtgccaagcgaagaggtggttaaaaagatgaagaactactggaggcaactgctcaacgcgaaattgatcacccagagaaagttcgataacctgaccaaggccgagaggggcggactctccgaacttgacaaagcgggcttcataaagaggcagctggtcgagacccgacagatcacgaagcacgtggcccaaatcctcgacagcagaatgaataccaagtacgatgagaatgacaaactcatcagggaagtgaaagtgattaccctgaagagcaagttggtgtccgactttcgcaaagatttccagttctacaaggtgagggagatcaacaactaccaccatgcccacgacgcatacctgaacgccgtggtcggcaccgccctgattaagaagtatccaaagctggagtccgaatttgtctacggcgactacaaagtttacgatgtgaggaagatgatcgctaagagcgaacaggagatcggcaaggccaccgctaagtatttcttctacagcaacatcatgaactttttcaagaccgagatcacacttgccaacggcgaaatcaggaagaggccgcttatcgagaccaacggtgagaccggcgagatcgtgtgggacaagggcagggacttcgccaccgtgaggaaagtcctgagcatgccccaggtgaatattgtgaaaaaaactgaggtgcagacaggcggctttagcaaggaatccatcctgcccaagaggaacagcgacaagctgatcgcccggaagaaggactgggaccctaagaagtatggaggcttcgacagccccaccgtagcctacagcgtgctggtggtcgcgaaggtagagaaggggaagagcaagaaactgaagagcgtgaaggagctgctcggcataaccatcatggagaggtccagctttgagaagaaccccattgactttttggaagccaagggctacaaagaggtcaaaaaggacctgatcatcaaactccccaagtactccctgtttgaattggagaacggcagaaagaggatgctggcgagcgctggggaactgcaaaagggcaacgaactggcgctgcccagcaagtacgtgaattttctgtacctggcgtcccactacgaaaagctgaaaggcagccccgaggacaacgagcagaagcagctgttcgtggagcagcacaagcattacctggacgagataatcgagcaaatcagcgagttcagcaagagggtgattctggccgacgcgaacctggataaggtcctcagcgcctacaacaagcaccgagacaaacccatcagggagcaggccgagaatatcatacacctgttcaccctgacaaatctgggcgcacctgcggcattcaaatacttcgataccaccatcgacaggaaaaggtacactagcactaaggaggtgctggatgccaccttgatccaccagtccattaccggcctgtatgagaccaggatcgacctgagccagcttggaggcgactctagggcggacccaaaaaagaaaaggaaggtggaattctctagaggcagtggagagggcagaggaagtctgctaacatgcggtgacgtcgaggagaatcctggcccaatgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaacttccccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagcttgaaactggagaacaaggaggcaaaggtctcgaagcgggagaaggcggtgtgggtgctgaaccctgaggcggggatgtggcagtgtctgctgagtgactcgggacaggtcctgctggaatccaacatcaaggttctgcccacatggtcgaccccggtgcagccaatggccctgattgtgctggggggcgtcgccggcctcctgcttttcattgggctaggcatcttcttctgtgtcaggtgccggcacaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnngttttagagctagaaatagcaagttaaaataaggctagtccgttatcaacttgaaaaagtggcaccgagtcggtgcttttttctagtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 35 - DNA, 9220 bp, Artificial Sequence
  source: /note=“Description of Artificial Sequence Synthetic polynucleotide”
  modified_base (6733)..(6752): a, c, t, g, unknown or other
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggagcggccgccaccatgggcaagcccatccctaaccccctgttggggctggacagcaccgctcccaaaaagaaaaggaaggtgggcattcacggcgtgcctgcggccgacaaaaagtacagcatcggccttgatatcggcaccaatagcgtgggctgggccgttatcacagacgaatacaaggtacccagcaagaagttcaaggtgctggggaatacagacaggcactctatcaagaaaaaccttatcggggctctgctgtttgactcaggcgagaccgccgaggccaccaggttgaagaggaccgcaaggcgaaggtacacccggaggaagaacaggatctgctatctgcaggagatcttcagcaacgagatggccaaggtggacgacagcttcttccacaggctggaggagagcttccttgtcgaggaggataagaagcacgaacgacaccccatcttcggcaacatagtcgacgaggtcgcttatcacgagaagtaccccaccatctaccacctgcgaaagaaattggtggatagcaccgataaagccgacttgcgacttatctacttggctctggcgcacatgattaagttcaggggccacttcctgatcgagggcgaccttaaccccgacaacagtgacgtagacaaattgttcatccagcttgtacagacctataaccagctgttcgaggaaaaccctattaacgccagcggggtggatgcgaaggccatacttagcgccaggctgagcaaaagcaggcgcttggagaacctgatagcccagctgcccggtgaaaagaagaacggcctcttcggtaatctgattgccctgagcctgggcctgacccccaacttcaagagcaacttcgacctggcagaagatgccaagctgcagttgagtaaggacacctatgacgacgacttggacaatctgctcgcccaaatcggcgaccagtacgctgacctgttcctcgccgccaagaacctttctgacgcaatcctgcttagcgatatccttagggtgaacacagagatcaccaaggcccccctgagcgccagcatgatcaagaggtacgacgagcaccatcaggacctgacccttctgaaggccctggtgaggcagcaactgcccgagaagtacaaggagatctttttcgaccagagcaagaacggctacgccggctacatcgacggcggagccagccaagaggagttctacaagttcatcaagcccatcctggagaagatggatggcaccgaggagctgctggtgaagctgaacagggaagatttgctccggaagcagaggacctttgacaacggtagcatcccccaccagatccacctgggcgagctgcacgcaatactgaggcgacaggaggatttctaccccttcctcaaggacaatagggagaaaatcgaaaagattctgaccttcaggatcccctactacgtgggccctcttgccaggggcaacagccgattcgcttggatgacaagaaagagcgaggagaccatcaccccctggaacttcgaggaagtggtggacaaaggagcaagcgcgcagtctttcatcgaacggatgaccaatttcgacaaaaacctgcctaacgagaaggtgctgcccaagcacagcctgctttacgagtacttcaccgtgtacaacgagctcaccaaggtgaaatatgtgaccgagggcatgcgaaaacccgctttcctgagcggcgagcagaagaaggccatcgtggacctgctgttcaagaccaacaggaaggtgaccgtgaagcagctgaaggaggactacttcaagaagatcgagtgctttgatagcgtggaaataagcggcgtggaggacaggttcaacgccagcctgggcacctaccacgacttgttgaagataatcaaagacaaggatttcctggataatgaggagaacgaggatatactcgaggacatcgtgctgactttgaccctgtttgaggaccgagagatgattgaagaaaggctcaaaacctacgcccacctgttcgacgacaaagtgatgaaacaactgaagagacgaagatacaccggctggggcagactgtccaggaagctcatcaacggcattagggacaagcagagcggcaagaccatcctggatttcctgaagtccgacggcttcgccaaccgaaacttcatgcagctgattcacgatgacagcttgaccttcaaggaggacatccagaaggcccaggttagcggccagggcgactccctgcacgaacatattgcaaacctggcaggctcccctgcgatcaagaagggcatactgcagaccgttaaggttgtggacgaattggtcaaggtcatgggcaggcacaagcccgaaaacatagttatagagatggccagagagaaccagaccacccaaaagggccagaagaacagccgggagcgcatgaaaaggatcgaggagggtatcaaggaactcggaagccagatcctcaaagagcaccccgtggagaatacccagctccagaacgagaagctgtacctgtactacctgcagaacggcagggacatgtacgttgaccaggagttggacatcaacaggctttcagactatgacgtggatcacatagtgccccagagctttcttaaagacgatagcatcgacaacaaggtcctgacccgctccgacaaaaacaggggcaaaagcgacaacgtgccaagcgaagaggtggttaaaaagatgaagaactactggaggcaactgctcaacgcgaaattgatcacccagagaaagttcgataacctgaccaaggccgagaggggcggactctccgaacttgacaaagcgggcttcataaagaggcagctggtcgagacccgacagatcacgaagcacgtggcccaaatcctcgacagcagaatgaataccaagtacgatgagaatgacaaactcatcagggaagtgaaagtgattaccctgaagagcaagttggtgtccgactttcgcaaagatttccagttctacaaggtgagggagatcaacaactaccaccatgcccacgacgcatacctgaacgccgtggtcggcaccgccctgattaagaagtatccaaagctggagtccgaatttgtctacggcgactacaaagtttacgatgtgaggaagatgatcgctaagagcgaacaggagatcggcaaggccaccgctaagtatttcttctacagcaacatcatgaactttttcaagaccgagatcacacttgccaacggcgaaatcaggaagaggccgcttatcgagaccaacggtgagaccggcgagatcgtgtgggacaagggcagggacttcgccaccgtgaggaaagtcctgagcatgccccaggtgaatattgtgaaaaaaactgaggtgcagacaggcggctttagcaaggaatccatcctgcccaagaggaacagcgacaagctgatcgcccggaagaaggactgggaccctaagaagtatggaggcttcgacagccccaccgtagcctacagcgtgctggtggtcgcgaaggtagagaaggggaagagcaagaaactgaagagcgtgaaggagctgctcggcataaccatcatggagaggtccagctttgagaagaaccccattgactttttggaagccaagggctacaaagaggtcaaaaaggacctgatcatcaaactccccaagtactccctgtttgaattggagaacggcagaaagaggatgctggcgagcgctggggaactgcaaaagggcaacgaactggcgctgcccagcaagtacgtgaattttctgtacctggcgtcccactacgaaaagctgaaaggcagccccgaggacaacgagcagaagcagctgttcgtggagcagcacaagcattacctggacgagataatcgagcaaatcagcgagttcagcaagagggtgattctggccgacgcgaacctggataaggtcctcagcgcctacaacaagcaccgagacaaacccatcagggagcaggccgagaatatcatacacctgttcaccctgacaaatctgggcgcacctgcggcattcaaatacttcgataccaccatcgacaggaaaaggtacactagcactaaggaggtgctggatgccaccttgatccaccagtccattaccggcctgtatgagaccaggatcgacctgagccagcttggaggcgactctagggcggacccaaaaaagaaaaggaaggtggaattctctagaggcagtggagagggcagaggaagtctgctaacatgcggtgacgtcgaggagaatcctggcccaatgaacctgagcaaaaacgtgagcgtgagcgtgtatatgaaggggaacgtcaacaatcatgagtttgagtacgacggggaaggtggtggtgatccttatacaggtaaatattccatgaagatgacgctacgtggtcaaaattccctacccttttcctatgatatcattaccacggcatttcagtatggtttccgcgtatttacaaaataccctgagggaattgttgactattttaaggactcgcttcccgacgcattccagtggaacagacgaattgtgtttgaagatggtggagtactaaacatgagcagtgatatcacatataaagataatgttctgcatggtgacgtcaaggctgagggagtgaacttcccgccgaatgggccagtgatgaagaatgaaattgtgatggaggaaccgactgaagaaacatttactccaaaaaacggggttcttgttggcttttgtcccaaagcgtacttacttaaagacggttcctattactatggaaatatgacaacattttacagatccaagaaatctggccaggcacctcctgggtatcactttgttaagcatcgtctcgtcaagaccaatgtgggacatggatttaagacggttgagcagactgaatatgccactgctcatgtcagtgatcttcccaagttcgaagcttgataatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnngttttagagctagaaatagcaagttaaaataaggctagtccgttatcaacttgaaaaagtggcaccgagtcggtgcttttttctagtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 36 - DNA, 23 bp, Artificial Sequence
  source: /note=“Description of Artificial Sequence Synthetic oligonucleotide”
gaaattaatacgactcactatag

SEQ ID NO: 37 - DNA, 34 bp, Artificial Sequence
  source: /note=“Description of Artificial Sequence Synthetic oligonucleotide”
ccctggcccaccctcgtgaccaccttcacctacg

SEQ ID NO: 38 - DNA, 34 bp, Artificial Sequence
  source: /note=“Description of Artificial Sequence Synthetic oligonucleotide”
gggaccgggtgggagcactggtggaagtggatgc

SEQ ID NO: 39 - DNA, 40 bp, Artificial Sequence
  source: /note=“Description of Artificial Sequence Synthetic oligonucleotide”
ccctggcccaccctcgtgaccaccttcacctacggcgtgc

SEQ ID NO: 40 - DNA, 40 bp, Artificial Sequence
  source: /note=“Description of Artificial Sequence Synthetic oligonucleotide”
gggcacgggaccgggtgggagcactggtggaagtggatgc

SEQ ID NO: 41 - DNA, 40 bp, Artificial Sequence
  source: /note=“Description of Artificial Sequence Synthetic oligonucleotide”
cccgtgccctggcccaccctcgtgaccaccttcacctacg

SEQ ID NO: 42 - DNA, 40 bp, Artificial Sequence
  source: /note=“Description of Artificial Sequence Synthetic oligonucleotide”
gggaccgggtgggagcactggtggaagtggatgccgcacg

SEQ ID NO: 43 - DNA, 17 bp, Artificial Sequence
  source: /note=“Description of Artificial Sequence Synthetic oligonucleotide”
cttcacctacggcgtgc

SEQ ID NO: 44 - DNA, 19 bp, Artificial Sequence
  source: /note=“Description of Artificial Sequence Synthetic oligonucleotide”
cactggtggaagtggatgc

SEQ ID NO: 45 - DNA, 19 bp, Artificial Sequence
  source: /note=“Description of Artificial Sequence Synthetic oligonucleotide”
gtgaccaccttcacctacg

SEQ ID NO: 46 - DNA, 17 bp, Artificial Sequence
  source: /note=“Description of Artificial Sequence Synthetic oligonucleotide”
gaagtggatgccgcacg

SEQ ID NO: 47 - DNA, 53 bp, Artificial Sequence
  source: /note=“Description of Artificial Sequence Synthetic oligonucleotide”
caccggcaagctgcccgtgccctggcccaccctcgtgaccaccttcacctacg

SEQ ID NO: 48 - DNA, 53 bp, Artificial Sequence
  source: /note=“Description of Artificial Sequence Synthetic oligonucleotide”
gtggccgttcgacgggcacgggaccgggtgggagcactggtggaagtggatgc

SEQ ID NO: 49 - DNA, 59 bp, Artificial Sequence
  source: /note=“Description of Artificial Sequence Synthetic oligonucleotide”
caccggcaagctgcccgtgccctggcccaccctcgtgaccaccttcacctacggcgtgc

SEQ ID NO: 50 - DNA, 59 bp, Artificial Sequence
  source: /note=“Description of Artificial Sequence Synthetic oligonucleotide”
gacgtggtggccgttcgacgggcacgggaccgggtgggagcactggtggaagtggatgc

SEQ ID NO: 51 - DNA, 59 bp, Artificial Sequence
  source: /note=“Description of Artificial Sequence Synthetic oligonucleotide”
ctgcaccaccggcaagctgcccgtgccctggcccaccctcgtgaccaccttcacctacg

SEQ ID NO: 52 - DNA, 59 bp, Artificial Sequence
  source: /note=“Description of Artificial Sequence Synthetic oligonucleotide”
gtggccgttcgacgggcacgggaccgggtgggagcactggtggaagtggatgccgcacg

SEQ ID NO: 53 - DNA, 97 bp, Artificial Sequence
  source: /note=“Description of Artificial Sequence Synthetic oligonucleotide”
catctgcaccaccggcaagctgcccgtgccctggcccaccctcgtgaccaccttcacctacggcgtgcagtgcttcgcccgctaccccgaccacatg

SEQ ID NO: 54 - DNA, 400 bp, Artificial Sequence
  source: /note=“Description of Artificial Sequence Synthetic polynucleotide”
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggagg

SEQ ID NO: 55 - DNA, 100 bp, Artificial Sequence
  source: /note=“Description of Artificial Sequence Synthetic polynucleotide”
gcatttctcagtcctaaacagttttagagctagaaatagcaagttaaaataaggctagtccgttatcaacttgaaaaagtggcaccgagtcggtgctttt

SEQ ID NO: 56 - DNA, 18 bp, Artificial Sequence
  source: /note=“Description of Artificial Sequence Synthetic primer”
acatcagcagctgttctg

SEQ ID NO: 57 - DNA, 18 bp, Artificial Sequence
  source: /note=“Description of Artificial Sequence Synthetic primer”
ggctgaaaggagagaact

SEQ ID NO: 58 - DNA, 100 bp, Artificial Sequence
  source: /note=“Description of Artificial Sequence Synthetic polynucleotide”
actcgaaggtcacaaattcggttttagagctagaaatagcaagttaaaataaggctagtccgttatcaacttgaaaaagtggcaccgagtcggtgctttt

SEQ ID NO: 59 - DNA, 21 bp, Artificial Sequence
  source: /note=“Description of Artificial Sequence Synthetic primer”
gactctgagcccatctcttgg

SEQ ID NO: 60 - DNA, 20 bp, Artificial Sequence
  source: /note=“Description of Artificial Sequence Synthetic primer”
ccatcttcagtctcctcacc

SEQ ID NO: 61 - DNA, 100 bp, Artificial Sequence
  source: /note=“Description of Artificial Sequence Synthetic polynucleotide”
gtattgaacgatgtcttgaggttttagagctagaaatagcaagttaaaataaggctagtccgttatcaacttgaaaaagtggcaccgagtcggtgctttt

SEQ ID NO: 62 - DNA, 23 bp, Artificial Sequence
  source: /note=“Description of Artificial Sequence Synthetic primer”
gtttgaggcatgtaattcatacc

SEQ ID NO: 63 - DNA, 23 bp, Artificial Sequence
  source: /note=“Description of Artificial Sequence Synthetic primer”
gagctcacagaacactggatctg

SEQ ID NO: 64 - protein, 34 aa, Artificial Sequence
  source: /note=“Description of Artificial Sequence Synthetic polypeptide”
LTPEQVVAIASHDGGKQALETVQRLLPVLCQAHG

SEQ ID NO: 65 - DNA, 10 bp, Artificial Sequence
  source: /note=“Description of Artificial Sequence Synthetic oligonucleotide”
cacttacgga

SEQ ID NO: 66 - DNA, 11 bp, Artificial Sequence
  source: /note=“Description of Artificial Sequence Synthetic oligonucleotide”
tcgttcaacgc

SEQ ID NO: 67 - DNA, 10 bp, Artificial Sequence
  source: /note=“Description of Artificial Sequence Synthetic oligonucleotide”
aggctacggt

SEQ ID NO: 68 - DNA, 10 bp, Artificial Sequence
  source: /note=“Description of Artificial Sequence Synthetic oligonucleotide”
tagctaccta

(19) United States
(12) Patent Application Publication
(10) Pub. No.: US 2019/0390229 A1
(43) Pub. Date: Dec. 26, 2019

(54) GENE EDITING REAGENTS WITH REDUCED TOXICITY

(71) Applicant: LIFE TECHNOLOGIES CORPORATION, CARLSBAD, CA (US)

(72) Inventors: Robert Jason POTTER, San Marcos, CA (US); Jason CARTER, Carlsbad, CA (US)

(21) Appl. No.: 16/095,147

(22) PCT Filed: Apr. 20, 2017
(86) PCT No.: PCT/US2017/028482

§ 371 Date: Oct. 19, 2018

Related U.S. Application Data
(60) Provisional application No. 62/325,805, filed on Apr. 21, 2016.

(51) Int. Cl. C12N 15/90; C12N 15/11; C12N 9/22; C12P 19/34

(52) U.S. Cl. CPC C12N 15/907; C12N 15/11; C12N 2800/80; C12P 19/34; C12N 2310/20; C12N 9/22

(57) ABSTRACT

The present disclosure generally relates to compositions and methods for the genetic modification of cells. In particular, the disclosure relates to CRISPR reagents and the use of such reagents. Further disclosed are nucleic acid compositions with reduced cytotoxicity.

### RELATED APPLICATIONS

This application is a § 371 National Phase Application of International Application No. PCT/US2017/028482 filed on Apr. 20, 2017, which claims the benefit of U.S. Provisional Application No. 62/325,805 filed Apr. 21, 2016, each of which are hereby incorporated by reference in their entireties.

### SEQUENCE LISTING

This application hereby incorporates by reference the material of the electronic Sequence Listing filed concurrently herewith. The material in the electronic Sequence Listing is submitted as a text (.txt) file entitled LT01144PCT_SL.txt” created on May 9, 2017 which has a file size of 43 KB, and is herein incorporated by reference in its entirety.

### FIELD

The present disclosure generally relates to compositions and methods for the genetic modification of cells. In particular, the disclosure relates to CRISPR reagents and the use of such reagents. Further disclosed are nucleic acid compositions with reduced cytotoxicity.

### BACKGROUND

A number of genome-editing systems, such as designer zinc fingers, transcription activator-like effectors (TALEs), CRISPRs, and homing meganucleases, have been developed. One issue with these systems is that they require a both the identification of target sites for modification and the designing of a reagents specific for those sites, which is often laborious and time consuming. In one aspect, the invention allows for the efficient design, preparation, and use of genome editing reagents.

### SUMMARY

The present disclosure relates, in part, to compositions and methods for editing of nucleic acid molecules. There exists a substantial need for efficient systems and techniques for modifying genomes. This invention addresses this need and provides related advantages.

It has been found that dephosphorylation of termini of RNA molecules reduces the toxicity of these molecules. The invention relates, in part, to RNA molecules that do not contain terminal phosphate groups on one or both termini and, where appropriate, one or both strands. Such RNA molecules include RNAi molecules (e.g., shRNA, siRNA), guide RNA molecules, and mRNA molecules (e.g., mRNA encoding a Cas9 protein or a TAL effector protein).

CRISPR systems do not require the generation of customized proteins to target specific sequences but rather a single Cas enzyme that can be directed to a target nucleotide sequence (a target locus) by a short RNA molecule with sequence complementarity to the target. Some RNA molecules, however, are known to be toxic to cells.

The present disclosure is directed, in part, to gene editing system modifications that increase the usefulness of these systems. One problem associated with gene editing systems is the amount of time and labor required to design and produce target locus specific gene editing reagents. The invention provides, in part, compositions and methods for the efficient, cost-effective production of CRISPR components. The invention provides, in part, compositions and methods for gene editing within cells where the gene editing compositions exhibit high gene editing activity and/or low cytotoxicity.

In some specific aspects, the invention is directed to three types of sequence specific nucleic acid binding activities. Using the Cas9 proteins as an example, these three systems include those where Cas9 proteins are employed with (1) double-stranded cutting activity (e.g., one Cas9 protein gene editing systems), (2) nickase activity (e.g., two Cas9 protein gene editing systems, referred to as “dual nickase” systems), and (3) no cutting activity but with the retention of nucleic acid binding activity (e.g., “dead” Cas9, referred to as dCas9, useful, for example, for gene repression, gene activation, DNA methylation, etc.).

The invention includes methods for introducing one or more dephosphorylated RNA molecule into a cell, the methods comprising: (a) performing in vitro transcription on a DNA molecule to form an RNA molecule, (b) removing one or more terminal phosphate groups from the RNA molecule formed in (b) to produce a dephosphorylated RNA molecule, and (c) contacting a cell with the dephosphorylated RNA molecule under conditions that allow for uptake of the dephosphorylated RNA molecule by the cell, wherein the RNA molecule participates in gene editing or encodes a protein that participates in gene editing. In some instances, the RNA molecule is a guide RNA molecule or a messenger RNA molecule (e.g., a mRNA encoding a zinc finger protein, a TAL effector protein, a Cas9 protein, etc.). In many instances, the cell will be a eukaryotic cell such as a plant cell or an animal cell (e.g., a human cell, an insect cell, a mouse cell, etc.). Further, the dephosphorylated RNA molecule will often be contacted with the cell in the presence of a transfection reagent.

The invention also includes methods for producing one or more dephosphorylated RNA molecules, the methods comprising: (a) generating a DNA molecule by performing polymerase chain reactions (PCR) in a reaction mixture containing: (i) a double-stranded nucleic acid segment and (ii) at least one oligonucleotide capable of hybridizing to nucleic acid at one terminus of the double-stranded nucleic acid segment, wherein the DNA molecule is produced by the PCR reaction, and wherein the DNA molecule contains at or near one terminus a promoter suitable for in vitro transcription, (b) performing in vitro transcription to form an RNA molecule, and (c) removing one or more terminal phosphate groups from the RNA molecule formed in (b) to produce the dephosphorylated RNA molecule. In some instances, the nucleic acid molecule is produced by the PCR reaction encodes an RNA molecule from about 35 to about 150 (e.g., from about 35 to about 150, from about 70 to about 150, from about 35 to about 150, from about 50 to about 150, from about 35 to about 90, etc.) nucleotides in length. In addition, the nucleic acid molecule produced by the PCR reaction may encode an RNA molecule comprising at least two hairpin turns. In some instances, the nucleic acid molecule produced by the PCR reaction encodes a CRISPR RNA or a guide RNA.

The invention further includes methods for producing one or more dephosphorylated RNA molecule, the methods comprising: (a) performing polymerase chain reaction (PCR) in a reaction mixture comprising: (i) a double-stranded nucleic acid segment comprising a first terminus and a second terminus, (ii) a first oligonucleotide comprising a first terminus and a second terminus, wherein the second terminus of the first oligonucleotide is capable of hybridizing to the first terminus of the double-stranded nucleic acid segment, and (iii) a second oligonucleotide comprising a first terminus and a second terminus, wherein the second terminus of the second oligonucleotide is capable of hybridizing to the first terminus of the first oligonucleotide, to produce the nucleic acid molecule, wherein the product nucleic acid molecule contains a promoter suitable for in vitro transcription at or near one terminus and encodes a CRISPR RNA, (b) performing in vitro transcription to form an RNA molecule, and (c) removing one or more terminal phosphate groups from the RNA molecule formed in (b) to produce the dephosphorylated RNA molecule. Further, the nucleic acid molecule is produced by the PCR reaction may encode a guide RNA. In some instances, the reaction mixture further comprises a first primer and a second primer, wherein the first primer is capable of hybridizing at or near the first terminus of the second oligonucleotide and the second primer is capable of hybridizing at or near the second terminus of the double-stranded nucleic acid segment.

The invention also includes methods for producing one or more dephosphorylated RNA molecules, the methods comprising: (a) performing polymerase chain reaction in a reaction mixture containing: (i) a first double-stranded nucleic acid segment comprising a first terminus and a second terminus, (ii) a second double-stranded nucleic acid segment comprising a first terminus and a second terminus, and (iii) at least one oligonucleotide comprising a first terminus and a second terminus, wherein the first terminus of the oligonucleotide is capable of hybridizing to nucleic acid at the first terminus of the first double-stranded nucleic acid segment to produce the nucleic acid molecule, wherein the second terminus of the oligonucleotide is capable of hybridizing to nucleic acid at the second terminus of the second double-stranded nucleic acid segment to produce the nucleic acid molecule, and wherein the product nucleic acid molecule contains a promoter suitable for in vitro transcription at or near one terminus, (b) performing in vitro transcription to form an RNA molecule, and (c) removing one or more terminal phosphate groups from the RNA molecule formed in (b) to produce the dephosphorylated RNA molecule.

The invention further includes methods for producing one or more nucleic acid molecules, the methods comprising: (a) performing polymerase chain reaction in a reaction mixture containing: (i) a first double-stranded nucleic acid segment comprising a first terminus and a second terminus, (ii) a second double-stranded nucleic acid segment comprising a first terminus and a second terminus, (iii) a first oligonucleotide comprising a first terminus and a second terminus, and (iv) a second oligonucleotide comprising a first terminus and a second terminus, wherein the second terminus of the first oligonucleotide is capable of hybridizing to nucleic acid at the first terminus of the second double-stranded nucleic acid segment, wherein the second terminus of the second oligonucleotide is capable of hybridizing to the first terminus of the first oligonucleotide, wherein the second terminus of the second oligonucleotide is capable of hybridizing to the first terminus of the second double-stranded nucleic acid segment, and wherein the product nucleic acid molecule contains a promoter suitable for in vitro transcription at or near one terminus, (b) performing in vitro transcription to form an RNA molecule, and (c) removing one or more terminal phosphate groups from the RNA molecule formed in (b) to produce the dephosphorylated RNA molecule.

The invention additionally includes methods for producing a CRISPR RNA molecules, the methods comprising contacting two or more linear RNA segments with each other under conditions that allow for the 5′ terminus of a first RNA segment to be covalently linked with the 3′ terminus of a second RNA segment to form the CRISPR RNA molecules, wherein terminal phosphate groups are removed from one or both termini of the CRISPR RNA. Further, the CRISPR RNA molecule may be separated from reaction mixture components, for example, by high-performance liquid chromatography.

The invention also includes methods for producing a guide RNA molecule, the method comprising: (a) separately producing a crRNA molecule and a tracrRNA molecule, and (b) contacting the crRNA molecule and the tracrRNA molecule with each other under conditions that allow for the covalently linking of the 3′ terminus of the crRNA to the 5′ terminus of the tracrRNA to produce the guide RNA molecule, wherein terminal phosphate groups are removed from one or both termini of the guide RNA molecule. Further, the guide RNA molecule may have a region of sequence complementarity of at least 10 (e.g., from about 10 to about 30, from about 10 to about 25, from about 15 to about 30, from about 15 to about 25, etc.) nucleotides to a target locus. Additionally, the target locus may be a naturally occurring chromosomal locus in a eukaryotic cell.

The invention also includes compositions comprising two RNA molecules connected by a triazole group, wherein one of the RNA molecules has a region of sequence complementarity of at least 10 nucleotides to a target locus, wherein terminal phosphate groups are removed from one or both termini of the two RNA molecules connected by linking group (e.g., a triazole group).

The invention further includes methods for gene editing at target loci within cells, these methods comprising introducing into a cell at least one CRISPR protein and at least one CRISPR RNA molecule, wherein the at least one CRISPR RNA molecule has a region of sequence complementarity of at least 10 (e.g., from about 10 to about 30, from about 10 to about 25, from about 15 to about 30, from about 15 to about 25, etc.) base pairs to the target locus, and wherein terminal phosphate groups are removed from one or both termini of the at least one CRISPR RNA molecule. Further, a linear DNA segment that has sequence homology at both termini to the target locus may be also introduced into the cell. In some instances, the at least one CRISPR protein is Cas9 protein. The Cas9 protein may have the ability to make a double-stranded cut in DNA or, in some instances, two Cas9 proteins may be introduced into the cell and each Cas9 protein may have the ability to nick double-stranded DNA. Further, one of the Cas9 proteins may have a mutation that renders to HNH domain inactive and the other Cas9 protein may have a mutation that renders to RuvC domain rendering that domain inactive. Of course, either the HNH domain or the RuvC domain may be inactivated in both Cas9 proteins that interact at the cut site. Thus, only one Cas9 protein, could be used. Along such lines, two RNA molecules, each with sequence complementarity to different target sequences, are introduced into the cell. In some instances, the different target sequences may be located within twenty base pairs of each other.

The invention further includes compositions comprising a mixture of capped mRNA molecules and uncapped mRNA molecules having a hydroxyl group on the 5′ terminus, wherein less than 3% of the mRNA molecules in the mixture contain a phosphate group at the 5′ terminus. In some instances, less than 1% of the mRNA molecules in the mixture may contain a phosphate group at the 5′ terminus. Such compositions may further comprise a transfection reagent. In some instances, mRNA molecules in the mixture encode one or more of a Cas9 protein, a transcription activator-like effector protein, or a zinc finger protein. Further, such proteins may be fusion proteins. These fusion proteins may contain one or more nuclear localization signal and/or one or more heterologous nuclease domain, as well as other protein regions conferring one or more functional activity (e.g., transcriptional activation or repression, etc.).

The invention further includes methods for preparing populations of mRNA molecules, these methods comprising: (a) performing in vitro transcription to generate a mixture containing capped mRNA molecules, (b) treating the mixture generated in (a) with a phosphatase under condition suitable for the removal of 5′ phosphate groups from mRNA molecules present in the mixture. Further, the phosphatase may be calf intestinal alkaline phosphatase (CIAP).

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a representative diagram of some aspects of the invention. This diagram shows examples of reagents for single component (e.g., zinc finger and TAL systems) and multi-component gene altering systems (e.g., CRISPR systems, such as Cas9 and Cpf1 systems). Reagents for the representative single components systems may be DNA, mRNA, and protein. Any one or more of these may be introduced into cells for genome alteration. Reagents for the representative multi-components systems may be DNA, RNA, and/or protein. One or both of these may be introduced into cells for genome alteration. DNA and mRNA reagents enter the cells as precursors that are them converted into functional RNA (e.g., gRNA) or proteins (e.g., Cas9, Cpf1, zinc finger nuclease or TAL nuclease). RNA molecules may be dephosphorylated prior to contact with cells.

FIG. 2 shows an exemplary plate format for use in one aspect of the invention. The plate contains a 6 by 8 array of wells where each well is identified by a number and letter combination. Wells A,1 and A,6 contain no gene altering reagents and thus are control wells. RNA molecules present in such reagents may be in dephosphorylated form or may be dephosphorylated prior it being brought into contact with cells.

FIG. 3 is a schematic drawing of the modular structure of a representative naturally occurring TAL protein. This protein is composed of an amino terminal end (N), a central array comprising a variable number of 34-amino acid repeats indicated by ovals with hypervariable residues at positions 12 and 13 that determine base preference, and a carboxyl terminal end (C) comprising a nuclear localization signal (NLS) and a transcription activator (AD) domain.

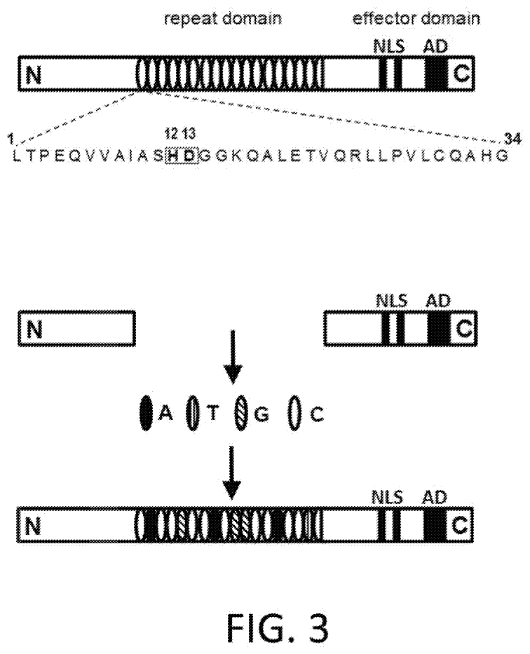

FIG. 4 is a representative diagram of a naturally occurring CRISPR system. In addition to the “Target DNA”, three additional components are required: Cas9 protein (shaded rectangle), crRNA (CRISPR RNA), and tracrRNA (trans-activating crRNA). The arrows labeled “RuvC and “HNH” indicate cutting locations in the Target DNA. The dashed box labeled “PAM” refers to protospacer adjacent motif. The 5′ termini of one or both of the CRISPR RNA and tracrRNA may be dephosphorylated.

FIG. 5 shows the association between a crRNA molecule (SEQ ID NO: 1) and a tracrRNA molecule (SEQ ID NO: 2). Hybridization Region 1 (19 nucleotides, in this instance) is complementary to the target site. Hybridization Region 2 is a region of sequence complementarity between the crRNA (41 nucleotides) and the tracrRNA (85 nucleotides). The tracrRNA 3′ region is the 3′ region of the tracrRNA molecule that extends beyond Hybridization Region 2. The Loop Replaceable Region is roughly defined by the closed box and may be replaced with a hairpin loop to connect crRNA and tracrRNA molecules into a single entity, typically referred to as a guide RNA.

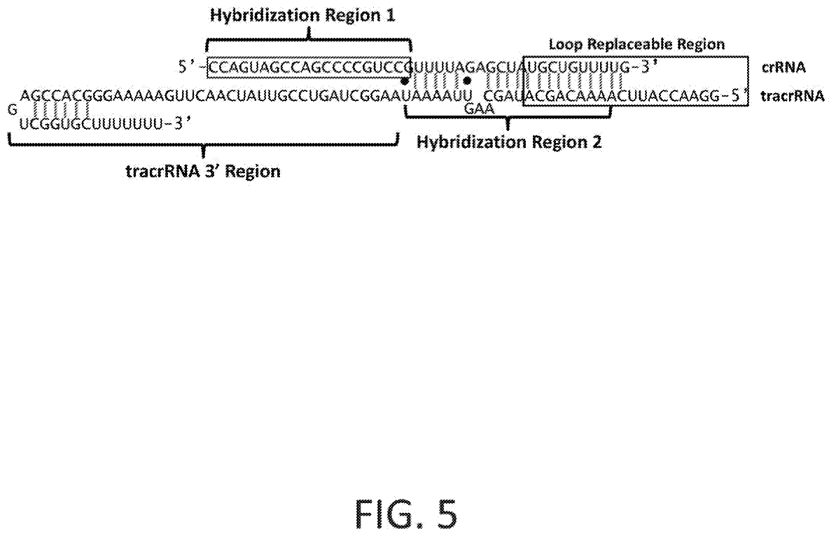

FIG. 6 is a schematic of a guide RNA molecule (104 nucleotides) showing the guide RNA bound to both Cas9 protein and a target genomic locus. Hairpin Region 1 is formed by the hybridization of complementary crRNA and tracrRNA regions joined by the nucleotides GAAA. Hairpin Region 2 is formed by a complementary region in the 3′ portion of the tracrRNA. FIG. 6 discloses SEQ ID NOs: 3-5, respectively, in order of appearance.

FIG. 7 is a schematic showing a nicking based nucleic acid cleavage strategy using a CRISPR system. In the top portion of the figure, two lines represent double-stranded nucleic acid. Two nick sites are indicated by Site 1 and Site 2. These sites are located within a solid or dashed box indicating the region of the nucleic acid that interacts with the CRISPR/Cas9 complex. The lower portion of the figure show nicking actions that result in two closely positioned nicks in both strands.

FIG. 8 is a schematic showing some methodologies for transient CRISPR activity within cells. The introduction of Cas9 proteins and/or nucleic acid encoding Cas9 is shown on the left. The introduction of guide RNA, crRNA plus tracrRNA, or crRNA alone is shown on the right. The middle shows the introduction of linear DNA encoding Cas9 or Cas9 plus tracrRNA. This DNA is designed to be stably maintained in the cell. The 5′ terminus of one or more of the various RNA molecules represented in this figure may be dephosphorylated.

FIG. 9 shows a workflow for synthesizing guide RNA using DNA oligo templates. Guide RNA encoding DNA template is generated using assembly PCR. Components of this assembly reaction include 1) a target specific DNA oligo (encodes the crRNA region), 2) DNA oligo specific to the bacterial promoter used for in vitro transcription (in this case T7 promoter), and 3) overlapping PCR products encoding tracrRNA region. A fill in reaction followed by PCR amplification is performed in a Thermo cycler using DNA polymerase enzyme (in this case high fidelity PHUSION® Taq DNA polymerase) to generate full length gRNA encoding templates. Following PCR assembly the resulting DNA template is transcribed at 37° C. to generate target specific gRNA using in vitro transcription reagents for non-coding RNA synthesis (in this case MEGASHORTSCRIPT™ T7 kit). Following synthesis the resulting gRNA is purified using a column or magnetic bead based method. After dephosphorylation, purified in vitro transcribed guide RNA is ready for co-transfection with Cas9 protein or mRNA delivery in a host system or cell line of interest. FIG. 9 guide RNA disclosed as SEQ ID NO: 3.

FIG. 10 shows overlapping DNA oligos as template for gRNA synthesis. The T7 promoter sequence and the overlap region are each about 20 nucleotides in length. Further, the box labeled “20 bp crRNA” is the target recognition component of the crRNA. Guide RNA is synthesized using 2 overlapping DNA oligonucleotides. 1) The forward DNA oligo contains the T7 promoter region (or other relevant in vitro transcription promoter), followed by target specific crRNA encoding region and a region that overlaps with the reverse oligonucleotide 2) Reverse DNA oligo encodes a tracrRNA sequence that is the constant component. These two overlapping oligonucleotides are annealed and extended to generate a DNA template for gRNA in vitro transcription (IVT) using high fidelity DNA polymerase enzyme (example PHUSION® Taq DNA polymerase). The assembly reaction also includes a 2-3 PCR cycling condition to enrich for the full length templates. The assembled DNA template is then used to generate guide RNA at 37° C. using in vitro transcription reagents for non-coding RNA synthesis (in this case MEGASHORTSCRIPT™ T7 kit was used). Following gRNA synthesis the product is purified using a column or, alternatively, using bead based purification methods. Further, one or both termini of the gRNA may be dephosphorylated.

FIG. 11 shows a PCR assembly based method for producing DNA molecules that encode guide RNA molecules. In this schematic, “Oligo 1” encodes a T7 promoter and part of Hybridization Region 1 and “Oligo 2” encode part of Hybridization Region 1 and has overlapping sequence with the “3′ PCR Segment”. PCR is then used for assembly of these overlapping fragments, followed by amplification using the 5′ and 3′primers, resulting in a double-stranded DNA molecule containing a T7 promoter operably connected to a target specific guide RNA coding sequence. RNA may be produced from this double-stranded DNA molecule by in vitro transcription. This RNA may then be dephosphorylated. FIG. 11 guide RNA disclosed as SEQ ID NO: 3.

FIG. 12 shows overlapping DNA oligos as template for gRNA synthesis. The T7 promoter sequence and the overlap region are each about 20 nucleotides in length. Further, the box labeled “20 bp crRNA” is the target recognition component of the crRNA. Guide RNA is synthesized using 2 overlapping DNA oligonucleotides. 1) The forward DNA oligo contains the T7 promoter region (or other relevant in vitro transcription promoter), followed by target specific crRNA encoding region and a region that overlaps with the reverse oligonucleotide 2) Reverse DNA oligo encodes a tracrRNA sequence that is the constant component. These two overlapping oligonucleotides are annealed and extended to generate a DNA template for gRNA in vitro transcription (IVT) using high fidelity DNA polymerase enzyme (example PHUSION® Taq DNA polymerase). The assembly reaction also includes a 2-3 PCR cycling condition to enrich for the full length templates. The assembled DNA template is then used to generate guide RNA at 37° C. using in vitro transcription reagents for non-coding RNA synthesis (in this case MEGASHORTSCRIPT™ T7 kit was used). Following gRNA synthesis the product is purified using a column or, alternatively, using bead based purification methods. gRNA generated as shown here may then be dephosphorylated.

FIG. 13 shows PCR assembly method for synthesizing guide RNA expressing templates by PCR assembly. This method can be used to introduce other promoters and terminators in the context of the guide RNA. In this schematic, the overlap region between “First Oligo” and “Second Oligo” encode “Hybridization Region 1”. The ˜ in the RNA polymerase III promoter region represents an unrepresented segment of the nucleic acid molecule because these promoters can be several hundred bases in length. The RNA polymerase III terminator sequence is not shown in this figure. The 5′ primer and 3′ primer sequences extend beyond termini the nucleic acid segments that they hybridize to indicate that primers may be used to add additional functionalities to the amplified nucleic acid molecules. The gRNA produced by IVT may then be dephosphorylated.

FIG. 14. Cell engineering workflow. On day 1, the researcher designs CRISPR targets and seeds cells. Synthesis of gRNA and cell transfection with Cas9 protein/gRNA complex (Cas9 RNP) are performed on day 2. Genome cleavage assays carried out on days 3-4.

FIG. 15 shows a collection of variable crRNA molecules and a constant tracrRNA molecule. A specific crRNA molecule (crRNA3 in this instance) may be selected and then linked to a tracrRNA molecule. Typically, the individual resulting RNA molecules will be dephosphorylated, if appropriate (e.g., if generated by IVT).

FIG. 16 shows an exemplary method for linking two RNA segments. The linking reaction shown in this figure using propargyl on one terminus and azide on the other terminus is unidirectional in that the termini with the chemical modifications are the only one that can link with each other. Typically, the linked RNA molecules will be in dephosphorylated form.

FIG. 17 shows data from in vivo genome cleavage and detection assays. Gel Image A: Original cleavage assay with gRNA amplified from a plasmid versus gRNA assembled from 6 overlapping oligos. Less than 50% cleavage activity compared to plasmid is seen. This is due to incorrect assembly. Gel Image B: Assembly method as outlined in FIG. 11, with either a 20 bp overlap or 15 bp overlap. An equivalent cleavage activity compared to the plasmid control is seen. RNA molecules used in the assay from which these data were generated were not dephosphorylated.

FIG. 18 shows that CIAP treatment reduces toxicity in U2OS cells. U2OS cells (about 10,000) were transfected with 125 ng Cas9 mRNA, either (−)untreated phosphorylated or (+)dephosphorylated with CIAP, 20 ng gRNA (or RNP (100 ng Cas9 protein), where indicated) with 0.3 ml LIPOFECTAMINE® RNAiMAX in duplicate in 96 well format. Viability determined by PRESTOBLUE™ 48 hours post-transfection followed by GCD.

FIG. 19 was produced using data generated essentially as set out in the FIG. 18 legend and shows that CIAP treatment enhances genome editing.

FIG. 20 shows U2OS cell viability after various treatments related to the HPRT gene target with CRISPR system components. “Un” refers untreated cells (i.e., control cells), “Alone” refer to either Cas9 protein or Cas9 mRNA without gRNA, “IVT” refers to in vitro transcribed gRNA, and “IVT+CIAP” refers to in vitro transcribed gRNA treated with calf intestinal alkaline phosphatase.

FIG. 21 shows U2OS cell viability after various treatments related to the PRKCG gene target with CRISPR system components. Labels are as in FIG. 20, with the addition of “syn” referring to the control of a synthetic gRNA which does has a 5′-OH.

FIG. 22 shows U2OS cell viability after various treatments related to the CMPK1 gene target with CRISPR system components. Labels are as in FIG. 21.

FIG. 23 shows gene editing efficiency in U2OS of CRISPR system components for the HPRT gene. “IVT” refer in vitro transcribed gRNA, and “IVT+CIAP” refer in vitro transcribed gRNA treated with calf intestinal alkaline phosphatase. “Syn” in later slides refers to chemically produced gRNA.

FIG. 24 shows gene editing efficiency in U2OS of CRISPR system components for the PRKCG gene. Labels are as in FIG. 23.

FIG. 25 shows gene editing efficiency in U2OS of CRISPR system components for the CMPK1 gene. Labels are as in FIG. 23.

FIG. 26 shows A549 cell viability after various treatments related to the HPRT gene with CRISPR system components. Labels are as in FIG. 20.

FIG. 27 shows A549 cell viability after various treatments related to the PRKCG gene with CRISPR system components. Labels are as in FIG. 21.

FIG. 28 shows A549 cell viability after various treatments related to the CMPK1 gene with CRISPR system components. Labels are as in FIG. 21.

FIG. 29 shows gene editing efficiency in A549 of CRISPR system components for the HPRT gene. Labels are as in FIG. 23.

FIG. 30 shows gene editing efficiency in A549 of CRISPR system components for the PRKCG gene. Labels are as in FIG. 23.

FIG. 31 shows gene editing efficiency in A549 of CRISPR system components for the CMPK1 gene. Labels are as in FIG. 23.

### DETAILED DESCRIPTION

### Definitions

As used herein the term “CRISPR activity” refers to an activity associated with a CRISPR system. Examples of such activities are double-stranded nuclease, nickase, transcriptional activation, transcriptional repression, nucleic acid methylation, nucleic acid demethylation, and recombinase.

As used herein the term “CRISPR system” refers to a collection of CRISPR proteins and nucleic acid that, when combined, result in at least CRISPR associated activity (e.g., the target locus specific, double-stranded cleavage of double-stranded DNA).

As used herein the term “CRISPR complex” refers to the CRISPR proteins and nucleic acid (e.g., RNA) that associate with each other to form an aggregate that has functional activity. An example of a CRISPR complex is a wild-type Cas9 (sometimes referred to as Csn1) protein that is bound to a guide RNA specific for a target locus.

As used herein the term “CRISPR protein” refers to a protein comprising a nucleic acid (e.g., RNA) binding domain nucleic acid and an effector domain (e.g., Cas9, such as Streptococcus pyogenes Cas9). The nucleic acid binding domains interact with a first nucleic acid molecules either having a region capable of hybridizing to a desired target nucleic acid (e.g., a guide RNA) or allows for the association with a second nucleic acid having a region capable of hybridizing to the desired target nucleic acid (e.g., a crRNA). CRISPR proteins can also comprise nuclease domains (i.e., DNase or RNase domains), additional DNA binding domains, helicase domains, protein-protein interaction domains, dimerization domains, as well as other domains.

CRISPR protein also refers to proteins that form a complex that binds the first nucleic acid molecule referred to above. Thus, one CRISPR protein may bind to, for example, a guide RNA and another protein may have endonuclease activity. These are all considered to be CRISPR proteins because they function as part of a complex that performs the same functions as a single protein such as Cas9.

In many instances, CRISPR proteins will contain nuclear localization signals (NLS) that allow them to be transported to the nucleus.

As used herein, the term “transcriptional regulatory sequence” refers to a functional stretch of nucleotides contained on a nucleic acid molecule, in any configuration or geometry, that act to regulate the transcription of (1) one or more structural genes (e.g., two, three, four, five, seven, ten, etc.) into messenger RNA or (2) one or more genes into untranslated RNA. Examples of transcriptional regulatory sequences include, but are not limited to, promoters, enhancers, repressors, and the like.

As used herein, the term “promoter” is an example of a transcriptional regulatory sequence, and is specifically a nucleic acid generally described as the 5′ region of a gene located proximal to the start codon or nucleic acid which encodes untranslated RNA. The transcription of an adjacent nucleic acid segment is initiated at the promoter region. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

As used herein the term “nucleic acid targeting capability” refers to the ability of a molecule or a complex of molecule to recognize and/or associate with nucleic acid on a sequence specific basis. As an example, Hybridization Region 1 on a crRNA molecule confers nucleic acid targeting capability upon a CRISPR complex.

As used herein the term “target locus” refers to a site within a nucleic acid molecule for CRISPR system interaction (e.g., binding and cleavage). When a single CRISPR complex is designed to cleave double-stranded nucleic acid, then the target locus is the cut site and the surrounding region recognized by the CRISPR complex. When two CRISPR complexes are designed to nick double-stranded nucleic acid in close proximity to create a double-stranded break, then the region surrounding and including the break point is referred to as the target locus.

As used herein the term “nucleic acid alterations” refers to alteration or changes to genetic code or non-code based nucleic acid modifications. Genetic code alteration refers nucleotide sequence changes of nucleic acid molecules. Non-code based nucleic acid alteration refers to nucleic acid modifications, such as methylation, that do not involve nucleotide sequence alterations, as well as modifications that result in alteration of gene expression (e.g., histone acetylation, promoter activation, promoter repression, etc.). Thus, a functional TAL-VP16 fusion protein would result in non-code based nucleic acid alteration when involved in the transcription of DNA.

As used herein the term “gene altering reagent” refers a composition that has one or more nucleic acid alteration activity or contains a component of a complex that has one or more nucleic acid alteration activity. Exemplary gene altering reagents are reagents that contain functional zinc finger-FokI fusion proteins, functional TAL-VP16 fusion protein, and gRNA molecules that are capable of directing a Cas9 protein a specific nucleotide region of a target nucleic acid molecule.

As used herein the term “stabilized gene altering reagent” refers a reagent that may be stored for a period of time with minimal loss of functional activity. Parameters related to this definition are set out herein.

As used herein, the terms “vector” refers to a nucleic acid molecule (e.g., DNA) that provides a useful biological or biochemical property to an insert. Examples include plasmids, phages, autonomously replicating sequences (ARS), centromeres, and other sequences which are able to replicate or be replicated in vitro or in a host cell, or to convey a desired nucleic acid segment to a desired location within a host cell. A vector can have one or more restriction endonuclease recognition sites (e.g., two, three, four, five, seven, ten, etc.) at which the sequences can be cut in a determinable fashion without loss of an essential biological function of the vector, and into which a nucleic acid fragment can be spliced in order to bring about its replication and cloning.

As used herein the term “target locus” refers to a site within a nucleic acid molecule for gene altering reagent interaction (e.g., binding and cleavage). When the gene altering reagent is designed to cleave double-stranded nucleic acid, then the target locus is the cut site and the surrounding region recognized by the CRISPR complex. When the gene altering reagent is designed to nick double-stranded nucleic acid in close proximity to create a double-stranded break, then the region surrounding and including the break point is referred to as the target locus.

### Overview:

The invention relates, in part, to compositions and methods for the preparation of nucleic acid molecules. In part, the invention relates to combinations of proteins and nucleic acid molecules designed to interact with other nucleic acid molecules. As an example, the invention relates to protein nucleic acid complexes, where the nucleic acid component has sequence complementarity to a target nucleic acid molecule. In these systems, sequence complementarity between the complexed nucleic acid and the target nucleic acid molecule is used to bring the complex into association with the target nucleic acid. Once this occurs, functional activities associated with the complex may be used to modify the target nucleic acid molecule. In many instances, nucleic acid molecules prepared by and used in methods of the invention will be dephosphorylated (e.g., by contact with a phosphatase under suitable conditions to result in dephosphorylation of the nucleic acid molecules).

In some aspects, the invention includes the preparing use of and introduction into cells of nucleic acid molecules that exhibit low levels of cytotoxicity. These nucleic acid molecules have particular groups on one or both termini. These nucleic acid molecules further lack particular groups on one or both termini. This aspect of the invention is exemplified by RNA molecules. mRNA molecules, for example, may be produced by in vitro transcription (IVT), then introduced into cell where they are translated to produce proteins. Such mRNA molecules may contain a 5′ cap. The capping process, however, is typically not 100% efficient, resulting in the formation of some mRNA molecules having 5′ phosphate groups. The T7 RNA polymerase used in the IVT reaction to generate RNA leaves a 5′-triphosphate. Chemically synthesized gRNAs do not have a 5′ triphosphate and have been observed to have lower toxicity. While not wishing to be bound by theory, we hypothesized that this 5′triphosphate can be recognized by mammalian cells as foreign and elicit an immune response.

Further, the presence of 5′ phosphate groups on the termini of mRNA molecules can result in decreased cellular viability (e.g., mammalian cell viability) when mRNA molecules containing such termini are introduced into cells. Thus, increased cellular toxicity is associated with the introduction into cells of RNA molecules that contain 5′ phosphate groups.

The invention includes methods for preparing RNA molecules that do not contain 5′ terminal phosphate groups, as well as methods for introducing such RNA molecules into cells.

The invention also relates, in part, to compositions and methods for the genome alteration. In particular, the invention relates to stabilized reagents and methods for producing and using such reagents. Stabilization may result from storage conditions (e.g., temperature, humidity, etc.) or from chemical characteristics of reagents (e.g., chemically modified nucleotides, buffers, presence of reducing agents, etc.) being stored. In many instances, one or more RNA molecules present in or added to such reagents will have been treated to remove one or more terminal phosphate groups (e.g., 5′ phosphate groups).

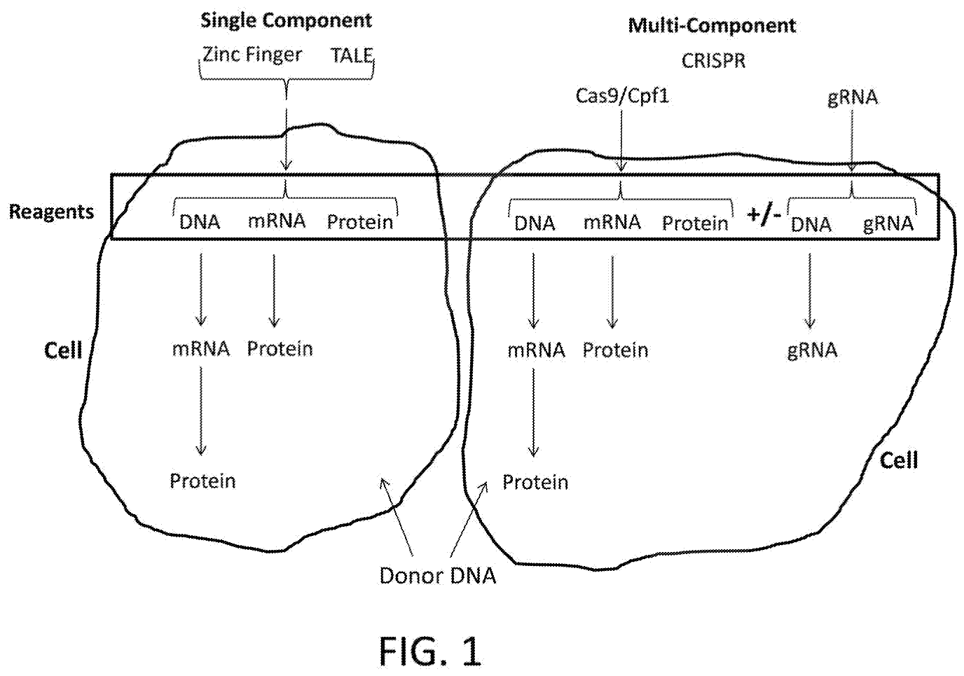

Using the schematic representation set out in FIG. 1 for purposes of illustration, two broad categories of gene altering reagents may be prepared: Single component and multi-component. Single component gene altering reagents refer to reagents that either are a gene alteration functional component or encode a gene alteration functional component. Thus, single component systems will typically comprise DNA, RNA, or protein. When the reagent is DNA, this DNA will typically be introduced into cells, where it is transcribed to form mRNA. The mRNA is then translated to generate protein as a gene alteration functional component (e.g., a zinc finger protein or a TAL protein).

Multi-component systems require more than one component for gene alteration activity. One example of this type of system is Cas9 based CRISPR systems. Systems such as this require a protein component (e.g., a Cas9 protein) and at least one nucleic acid component (e.g., a gRNA) for gene alteration activity. The protein component may be introduced into cells as a protein or encoded by mRNA or DNA that are introduced into the cell. Further, gRNA or DNA encoding gRNA may be introduced into cells that express one or more protein components of a multi-component system.

In most instances, the goal will be to either introduce into cells (1) one or more functional gene editing reagents (2) one or more nucleic acid molecule encoding gene editing reagents, or (3) a combination of one or more gene altering reagents that are ready to form gene altering complexes and one or more nucleic acid molecule encoding additional gene altering reagents.

In particular, the invention relates to combinations of proteins and nucleic acid molecules designed to interact with other nucleic acid molecules. In some instances, the invention relates to protein/nucleic acid complexes, where the nucleic acid component has sequence complementarity to a target nucleic acid molecule. In these systems, sequence complementarity between the complexed nucleic acid and the target nucleic acid molecule is the used to bring the complex into association with the target nucleic acid. Once this occurs, functional activities associated with the complex may be used to modify the target nucleic acid molecule.

Capped mRNA Molecules

The invention relates, in part, to mRNA molecules that exhibit low levels of cytotoxicity. One method of producing such mRNA molecules is by IVT under conditions that results in mRNA molecules being generated with 5′ caps.

One general workflow for the production of capped mRNA is as follows: Cap incorporation via IVT, DNAse treatment, polyA tailing reaction, and mRNA purification. A phosphatase reaction may be added to this workflow, for example, after mRNA purification. Further, an additional mRNA purification step may be added after the phosphatase reaction. Alternatively, the phosphatase reaction may occur as part of the original workflow or as a component of a step after the IVT/capping step.

One goal of the phosphatase reaction is to remove terminal phosphate groups (e.g., 5′ phosphate groups) to decrease cytotoxicity of the RNA molecules being generated. When capped mRNA molecules are generated, some of the RNA molecules in the mixture produced without caps. These uncapped mRNA molecules will often have 5′ terminal phosphate groups. Removal of these phosphate groups decreases the cytotoxicity of the mRNA mixture.

The invention thus include compositions and methods for decreasing the cytotoxicity of RNA molecules (e.g., mRNA molecules, gRNA molecules, rRNA molecules, shRNA molecules, etc.) generated by IVT, wherein the cytotoxicity is reduced by at least 30% (e.g., from about 30% to about 95%, from about 35% to about 95%, from about 40% to about 95%, from about 45% to about 95%, from about 50% to about 95%, from about 60% to about 95%, from about 30% to about 85%, from about 40% to about 85%, from about 50% to about 85%, etc.).

The invention thus include compositions and methods for decreasing the number of 5′ terminal phosphate groups of RNA molecules generated by IVT, wherein the number of 5′ terminal phosphate groups present in a composition is reduced by at least 30% (e.g., from about 30% to about 95%, from about 35% to about 95%, from about 40% to about 95%, from about 45% to about 95%, from about 50% to about 95%, from about 60% to about 95%, from about 30% to about 85%, from about 40% to about 85%, from about 50% to about 85%, etc.).

RNA caps that may be used in the practice of the invention include Anti-Reverse Cap Analog (ARCA, New England Biolabs, cat. no. S1411), and caps having the following structures: m7G(5′)ppp(5′)G, G(5′)ppp(5′)G, m7G(5′)ppp(5′)A, and G(5′)ppp(5′)A.

Phosphatases that may be used in the practice of the invention include alkaline phosphatases (e.g., calf intestinal alkaline phosphatase, shrimp alkaline phosphatase, chicken alkaline phosphatase, bacterial alkaline phosphatase, etc.) and Antarctic Phosphatase (New England Biolabs, cat. no. M0289).

### Exemplary Gene Altering Reagents:

Three different examples of gene altering systems are zinc finger based systems, TAL effectors based systems, CRISPR based systems (e.g., Cas9 based systems and CPF1 based systems). Each operates by different principles and employ different functional molecules. These systems break down into two groups: (1) Protein based systems (e.g., zinc finger and TAL effectors) and (2) nucleic acid/protein complexed based systems (e.g., CRISPRs).

### A. Zinc Finger Based Systems

Zinc-finger nucleases (ZFNs) and meganucleases are examples of genome engineering tools. ZFNs are chimeric proteins consisting of a zinc-finger DNA-binding domain and a nuclease domain. One example of a nuclease domain is the non-specific cleavage domain from the type IIS restriction endonuclease FokI (Kim, Y G; Cha, J., Chandrasegaran, S. Hybrid restriction enzymes: zinc finger fusions to Fok I cleavage domain Proc. Natl. Acad. Sci. USA. 1996 Feb. 6; 93(3):1156-60) typically separated by a linker sequence of 5-7 base pairs. A pair of the FokI cleavage domain is generally required to allow for dimerization of the domain and cleavage of a non-palindromic target sequence from opposite strands. The DNA-binding domains of individual Cys₂His₂ ZFNs typically contain between 3 and 6 individual zinc-finger repeats and can each recognize between 9 and 18 base pairs.

One problem associated with ZNFs is the possibility of off-target cleavage which may lead to random integration of donor DNA or result in chromosomal rearrangements or even cell death which still raises concern about applicability in higher organisms (Zinc-finger Nuclease-induced Gene Repair With Oligodeoxynucleotides: Wanted and Unwanted Target Locus Modifications Molecular Therapy vol. 18 no. 4, 743-753 (2010)).

### B. TAL Effectors Based Systems

Transcription activator-like (TAL) effectors represent a class of DNA binding proteins secreted by plant-pathogenic bacteria of the species, such as Xanthomonas and Ralstonia, via their type III secretion system upon infection of plant cells. Natural TAL effectors specifically have been shown to bind to plant promoter sequences thereby modulating gene expression and activating effector-specific host genes to facilitate bacterial propagation (Römer, P., et al., Plant pathogen recognition mediated by promoter activation of the pepper Bs3 resistance gene. Science 318, 645-648 (2007); Boch, J. & Bonas, U. Xanthomonas AvrBs3 family-type III effectors: discovery and function. Annu. Rev. Phytopathol. 48, 419-436 (2010); Kay, S., et al. U. A bacterial effector acts as a plant transcription factor and induces a cell size regulator. Science 318, 648-651 (2007); Kay, S. & Bonas, U. How Xanthomonas type III effectors manipulate the host plant. Curr. Opin. Microbiol. 12, 37-43 (2009)). Natural TAL effectors are generally characterized by a central repeat domain and a carboxyl-terminal nuclear localization signal sequence (NLS) and a transcriptional activation domain (AD). The central repeat domain typically consists of a variable amount of between 1.5 and 33.5 amino acid repeats that are usually 33-35 residues in length except for a generally shorter carboxyl-terminal repeat referred to as half-repeat. The repeats are mostly identical but differ in certain hypervariable residues. DNA recognition specificity of TAL effectors is mediated by hypervariable residues typically at positions 12 and 13 of each repeat—the so-called repeat variable diresidue (RVD) wherein each RVD targets a specific nucleotide in a given DNA sequence. Thus, the sequential order of repeats in a TAL protein tends to correlate with a defined linear order of nucleotides in a given DNA sequence. The underlying RVD code of some naturally occurring TAL effectors has been identified, allowing prediction of the sequential repeat order required to bind to a given DNA sequence (Boch, J. et al. Breaking the code of DNA binding specificity of TAL-type III effectors. Science 326, 1509-1512 (2009); Moscou, M. J. & Bogdanove, A. J. A simple cipher governs DNA recognition by TAL effectors. Science 326, 1501 (2009)). Further, TAL effectors generated with new repeat combinations have been shown to bind to target sequences predicted by this code. It has been shown that the target DNA sequence generally start with a 5′ thymine base to be recognized by the TAL protein.

The modular structure of TALs allows for combination of the DNA binding domain with effector molecules such as nucleases. In particular, TAL effector nucleases allow for the development of new genome engineering tools known.

### C. CRISPR Based Systems

Gene altering reagents may be based upon CRISPR systems. The term “CRISPR” is a general term that applies to three types of systems, and system sub-types. In general, the term CRISPR refers to the repetitive regions that encode CRISPR system components (e.g., encoded crRNAs). Three types of CRISPR systems (see Table 1) have been identified, each with differing features.

TABLE 1
CRISPR System Types Overview

| System | Features | Examples |
|---|---|---|
| Type I | Multiple proteins (5-7 proteins | Staphylococcus |
| | typical), crRNA, requires PAM. DNA | epidermidis |
| | Cleavage is catalyzed by Cas3. | (Type IA) |
| Type II | 3-4 proteins (one protein (Cas9) has | Streptococcus pyogenes |
| | nuclease activity) two RNAs, requires | CRISPR/Cas9, |
| | PAMs. Target DNA cleavage | Francisella novicida |
| | catalyzed by Cas9 and RNA | U112 Cpf1 |
| | components. | |
| Type III | Five or six proteins required for | S. epidermidis |
| | cutting, number of required RNAs | (Type IIIA); |
| | unknown but expected to be 1, PAMs | P. furiosus |
| | not required. Type IIIB systems have | (Type IIIB). |
| | the ability to target RNA. | |

While the invention has numerous aspects and variations associated with it, the Type II CRISPR/Cas9 system has been chosen as a point of reference for explanation herein.

In certain aspects, the invention provides stabilized crRNAs, tracrRNAs, and/or guide RNAs (gRNAs), as well as collections of such RNA molecules.

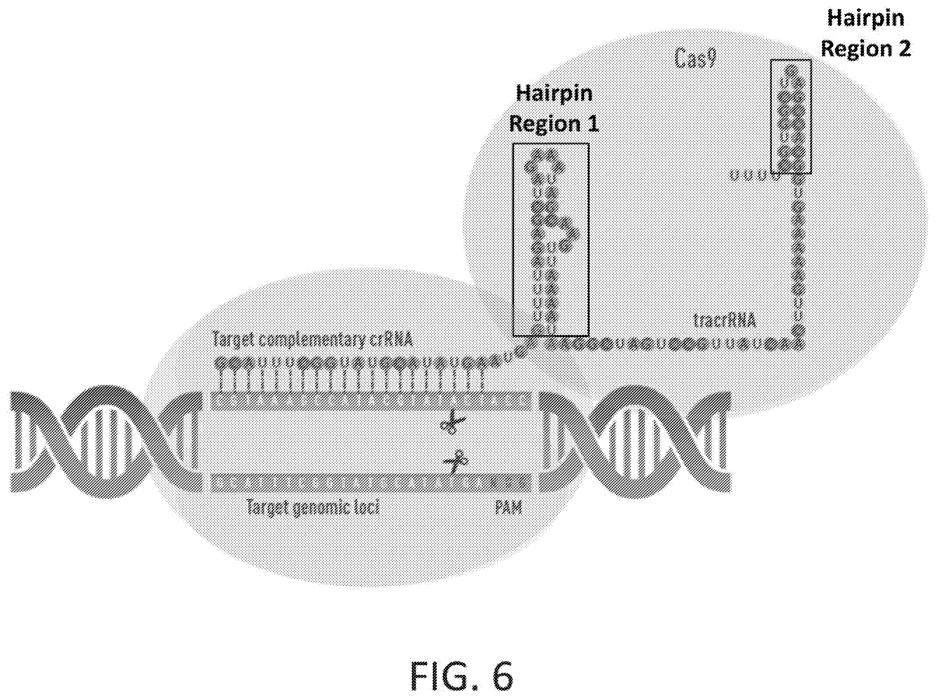

FIG. 6 shows components and molecular interactions associated with a Type II CRISPR system. In this instance, the Cas9 mediated Streptococcus pyogenes system is exemplified. A gRNA is shown in FIG. 6 hybridizing to both target DNA (Hybridization Region 1) and tracrRNA (Hybridization Region 2). In this system, these two RNA molecules serve to bring the Cas9 protein to the target DNA sequence is a manner that allows for cutting of the target DNA. The target DNA is cut at two sites, to form a double-stranded break.

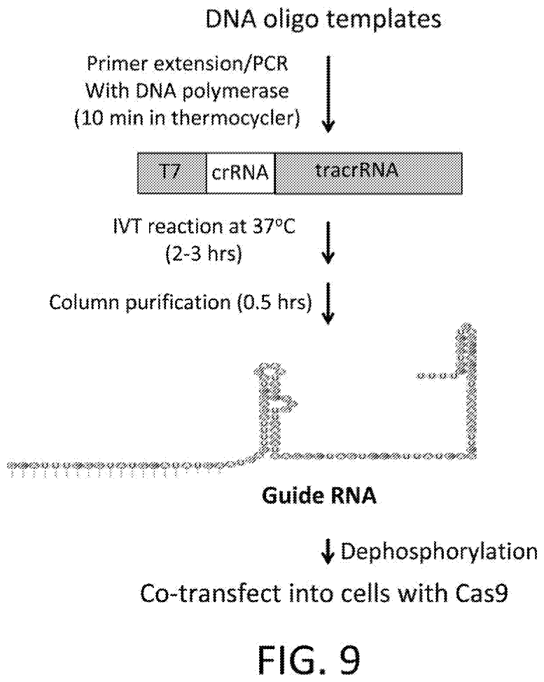

FIG. 9 shows an exemplary workflow of the invention. The schematic in FIG. 9 shows oligonucleotides designed to generate a DNA molecule where the guide RNA coding region is operably linked to a T7 promoter. In this work flow DNA oligonucleotides either alone or in conjunction with double-stranded DNA are used to generate, via PCR, a DNA molecule encoding a guide RNA operably linked to a promoter suitable for in vitro transcription. The DNA molecule is then transcribed in vitro to generate guide RNA. The guide RNA (gRNA) can be dephosphorylated to remove one or more phosphates from the 5′end. The guide RNA may then be “cleaned up” by, for example, column purification or bead based methods. The guide RNA is then suitable for use by, as examples, (1) direct introduction into a cell or (2) introduction into a cell after being complexed with one or more CRISPR protein. Nucleic acid operably connected to a T7 promoter can be transcribed in mammalian cells when these cells contain T7 RNA polymerase (Lieber et al., Nucleic Acids Res., 17: 8485-8493 (1989)). Of course, other promoters functional in eukaryotic cells (e.g., CMV promoter, U6 promoter, H1 promoter, etc.) could also be used for the intracellular production of guide RNA. The H1 promoter, for example, is about 300 base pairs in length. One advantage of the T7 promoter is its small size (20 base pairs).

One advantage of using chemically synthesized and in vitro transcribed RNA is that chemically modified bases may be introduced into the RNA molecules.

Dried or lyophilized gene altering complexes may also be used. A number of formulations may be used for dried or lyophilized gene altering reagents that have been allowed to form complexes. In many instances, complexes may be formed using CRISPR system reagents.

Dried or lyophilized gene altering reagents complexes may be tested and/or used by the introduction of such complexes in cells (e.g., U2OS cells, HEK293 cells, etc.). Further, complexes may be prepared in or placed into in multi-well formats in 1× to 5× amounts. For Cas9 mRNA formats, LIPOFECTAMINE® RNAiMAX, or equivalent, may be used. For Cas9 protein formats CRISPRMAX, or equivalent, may be used for lipid nanoparticle based transfection.

Cas9/gRNA are exemplary conditions are used below for purposes of illustration.

Format 1: No transfection reagent or Cas9. 1 to 5 μg of gRNA is added to wells of multiwell plates. The plate and contents is vacuum dried, then stored at different temperatures. Prior to use gRNA is resuspended to an appropriate concentration. Cas9 expressing stable cells or cells co-transfect with Cas9 and a suitable transfection reagent are added to the wells.

Format 2: 20 ng of IVT generated gRNA (20 ng/μl) and 100 ng Cas9mRNA (100 ng/μl) are mixed to form complexes and added to wells of multiwell plates. The plate and contents is vacuum dried, then stored at different temperatures. Prior to transfect, the samples are resuspended in RNAse and DNAse free water or OPTI-MEM™ culture medium. Following resuspension of the dried samples, LIPOFECTAMINE® RNAiMAX/OPTI-MEM™ mix (prepared using 0.60 of LIPOFECTAMINE® RNAiMAX and 4.40 OPTI-MEM™ per well) is added to the gRNA-Cas9 complexes and then applied to 15,000-20,000 cells/well.

Format 3: IVT gRNA (20 ng/well at 20 ng/μl) and Cas9 mRNA (100 ng/well at 100 ng/μl) is precomplexed with LIPOFECTAMINE® RNAiMAX (0.6 μl/well) and vacuum dry. Dried pre-complexed samples are resuspended in OPTI-MEM™ and used for transfection.

Format 4: IVT generated gRNA (20 ng/well) and Cas9 mRNA (100 ng/well) is precomplexed with LIPOFECTAMINE® RNAiMAX (or equivalent) and OPTI-MEM™ (4.40 per well). The mixture is vacuum dried. Prior to use samples are resuspended in OPTI-MEM™ and/or water and applied to cells in 96 well format.

Format 5: IVT generated gRNA is precomplexed with LIPOFECTAMINE® RNAiMAX or LIPOFECTAMINE® MESSENGERMAX™ (with or without OPTI-MEM™). This format may be used with stable Cas9 expressing cell lines. Amounts of components used are the same or similar to above described formats.

Format 6: Formats 1-4 with donor DNA (e.g., single-stranded DNA).

In certain aspects, the invention provides:

1. Individual oligonucleotides to make crRNA/tracrRNAs and collections of such oligonucleotides, as well as methods for generating and using such oligonucleotides.
2. Compositions and methods for introducing CRISPR complex components into cells.

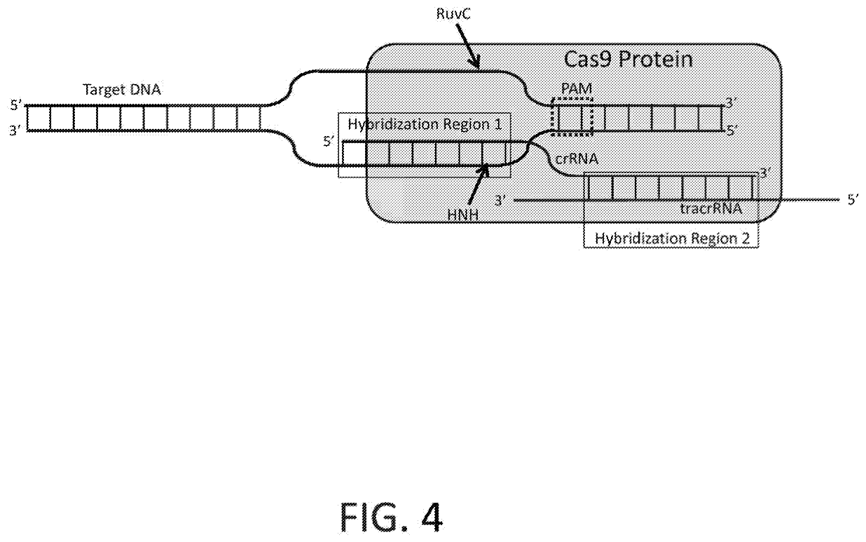

FIG. 4 shows components and molecular interactions associated with a Type II CRISPR system. In this instance, the Cas9 mediated Streptococcus pyogenes system is exemplified.

A crRNA is shown in FIG. 4 hybridizing to both target DNA (Hybridization Region 1) and tracrRNA (Hybridization Region 2). In this system, these two RNA molecules serve to bring the Cas9 protein to the target DNA sequence is a manner that allows for cutting of the target DNA. The target DNA is cut at two sites, to form a double-stranded break.

There appears to be substantial sequence variation in tracrRNA sequence. It has been postulated that tracrRNA function relates more to RNA structure, than RNA sequence.

The Cas9 protein of Streptococcus pyogenes is 1368 amino acids in length (NCBI Reference Sequence: WP_030126706.1) and contains a number of domains for the binding and cutting of nucleic acid molecules. This protein has two domains (RuvC and HNH), each of which has DNA nickase activity. When this protein nicks DNA on both strands, the nicks are in close enough proximity to result in the formation of a double-stranded break.

While not wishing to be bound by theory, in brief, as shown in FIG. 4, crRNA hybridizes to target DNA, referred to as “Hybridization Region 1”. Hybridization region 1 is typically in the range of 18 to 22 base pairs but can be longer or shorter. The crRNA thus “indentifies” the target DNA sequence. The crRNA also hybridizes to the tracrRNA, referred to as “Hybridization Region 2”. Hybridization Region 1 is typically in the range of 15 to 25 base pairs but can be longer or shorter and often there is not full sequence complementarity between the hybridized strands. The tracrRNA is believed to associate with the Cas9 protein, bringing the RuvC and HNH cleavage domains in contact with the target DNA.

A number of features of the CRISPR/Cas9 system, any or all of which may be used in the practice of the invention, have been identified:

1. crRNA and tracrRNA may be combined to form a guide RNA (gRNA).
2. Mutations may be introduced into Cas9 proteins that inactivate either the RuvC or HNH domains resulting in proteins with strand specific nickase activity.
3. Mutations may be introduced into Cas9 proteins that inactivate all nucleic acid cleavage activities but allow for these proteins to retain nucleic acid binding activity.
4. Sequence alterations, including truncations and multi-nucleotide deletions, can be made to the CRISPR system RNA components.

One limitation on Type II CRISPR systems is the requirement of a protospacer adjacent motif (PAM) for high level activity. Efficient binding and cleavage of DNA by Cas9-RNA requires recognition of a PAM. Typically, PAMs are three nucleotides in length.

In many instances, it will be desirable to make two nicks in close proximity to each other when cleaving nucleic acid using methods of the invention. This is especially so when the target locus is in a cellular genome. The use of CRISPR system components that nick nucleic acid is believed to limit “off-target effects” in that a single nick at a location other than the target locus is unlikely to result in single-stranded cleavage of the nucleic acid.

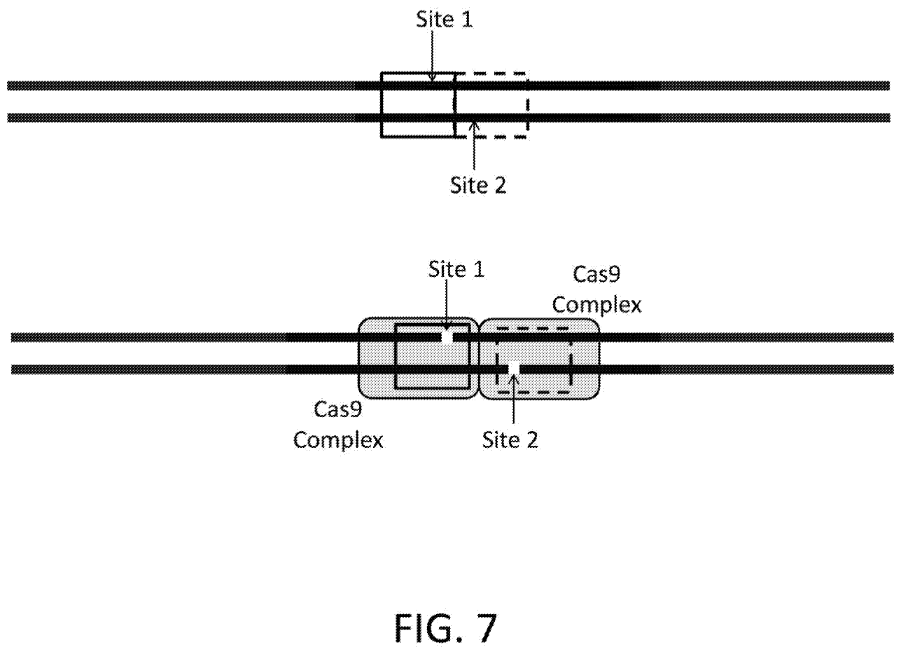

FIG. 7 shows the selection of two closely associated sites that form a target locus. Each of the sites (Site 1 and Site 2) binds a CRISPR complex with nickase activity.

The two sites exemplified in FIG. 7 will generally be located sufficiently close to each other so that the double-stranded nucleic acid containing the nick breaks. While this distance will vary with factors such as the AT/CG content of the region, the nick sites will generally be within 200 base pairs of each other (e.g., from about 1 to about 200, from about 10 to about 200, from about 25 to about 200, from about 40 to about 200, from about 50 to about 200, from about 60 to about 200, from about 1 to about 100, from about 10 to about 100, from about 20 to about 100, from about 30 to about 100, from about 40 to about 100, from about 50 to about 100, from about 1 to about 60, from about 10 to about 60, from about 20 to about 60, from about 30 to about 60, from about 40 to about 60, from about 1 to about 35, from about 5 to about 35, from about 10 to about 35, from about 20 to about 35, from about 25 to about 35, from about 1 to about 25, from about 10 to about 25, from about 15 to about 25, from about 2 to about 15, from about 5 to about 15, etc. base pairs).

In many instances, CRISPR complexes bind with high affinity to the target locus. In many such instances, when double-stranded breaks at the target locus are desired CRISPR complexes will be directed to the target locus in a manner such that they do not stericly interfere with each other. Thus, the invention includes methods in which CRISPR complex binding sites at a target locus are selected such that nicking activity on each strand is not significantly altered by the binding of a CRISPR complex directed to the nicking of the other strand. The invention further includes compositions for performing such methods.

TABLE 2
Predicted S. pyogenes Cas9 Functional Regions

| Description | Positions | Length |
|---|---|---|
| RuvC-I | 1-62 | 62 |
| Recognition lobe | 60-718 | 659 |
| RuvC-II | 718-765 | 48 |
| HNH | 810-872 | 63 |
| RuvC-III | 925-1102 | 178 |
| PAM-interacting domain | 1099-1368 | 270 |
| PAM substrate binding | 1125-1127 | 3 |

S. pyogenes Cas9 protein has a number of domains (see Table 2), two of which are nuclease domains. The discontinuous RuvC-like domain is encompassed by approximately amino acids 1-62, 718-765 and 925-1102. The HNH nuclease domain is encompassed by approximately amino acids residues 810-872. The recognition lobe, approximately amino acids 60-718, recognizes and binds regions of guide RNAs in a sequence-independent manner. Deletions of some parts of this lobe abolishes CRISPR activity. The PAM-interacting domain, approximately amino acids 1099-1368, recognizes the PAM motif.

The nicking activity may be accomplished in a number of ways. For example, the Cas9 protein has two domains, termed RuvC and HNH, that nick different strands of double-stranded nucleic acid. Cas9 proteins may be altered to inactivate one domain or the other. The result is that two Cas9 proteins are required to nick the target locus in order for a double-stranded break to occur. For example, an aspartate-to-alanine substitution (D10A) in the RuvC catalytic domain of Cas9 from S. pyogenes converts Cas9 from a nuclease that cleaves both strands to a nickase (cleaves a single strand). Other examples of mutations that render Cas9 a nickase include H840A, N854A, and N863A.

CRISPR proteins (e.g., Cas9) with nickase activities may be used in combination with guide sequences (e.g., two guide sequences) which target respectively sense and antisense strands of the DNA target.

Another way to generate double-stranded breaks in nucleic acid using nickase activity is by using CRISPR proteins that lack nuclease activity linked to a heterologous nuclease domain. One example of this is a mutated form of Cas9, referred to as dCas9, linked to FokI domain. FokI domains require dimerization for nuclease activity. Thus, in such instances, CRISPR RNA molecules are used to bring two dCas9-FokI fusion proteins into sufficiently close proximity to generate nuclease activity that results in the formation of a double-stranded cut. Methods of this type are set out in Tsai et al., “Dimeric CRISPR RNA-guided FokI nucleases for highly specific genome editing,” Nature Biotech., 32:569-576 (2014) and Guilinger et al., “Fusion of catalytically inactive Cas9 to FokI nuclease improves the specificity of genome modification,” Nature Biotech., 32:577-582 (2014).

### Transient Activity

One need is for a genome editing system having transient or highly regulatable activity. Transient activity is important for a number of applications. For example, for construction of cells lines involving one or more nuclease activity. Once a cellular nucleic acid, for example, has been effectively exposed to a nuclease and appropriately cut, repair of the nucleic acid (e.g., via non-homologous end-joining) normally takes place. Repair of the cellular nucleic acid is generally required for the cell to remain viable. In many cases, the cell will either integrate nucleic acid into the repaired nucleic acid molecule or nucleic acid will be removed (e.g., from 1 base pair to about 100 base pairs) for the repaired nucleic acid molecule. In either instance, a heritable change occurs within the genome of the cell. Cells with genetic changes can then be screened to identify ones with a desired alteration. Once cells with desired changes are identified, for most applications, it is beneficial to maintain the cells without further nuclease induced genetic change. Thus, it is generally desirable that the nuclease activity used to facilitate the genetic changes not be active within the cells.

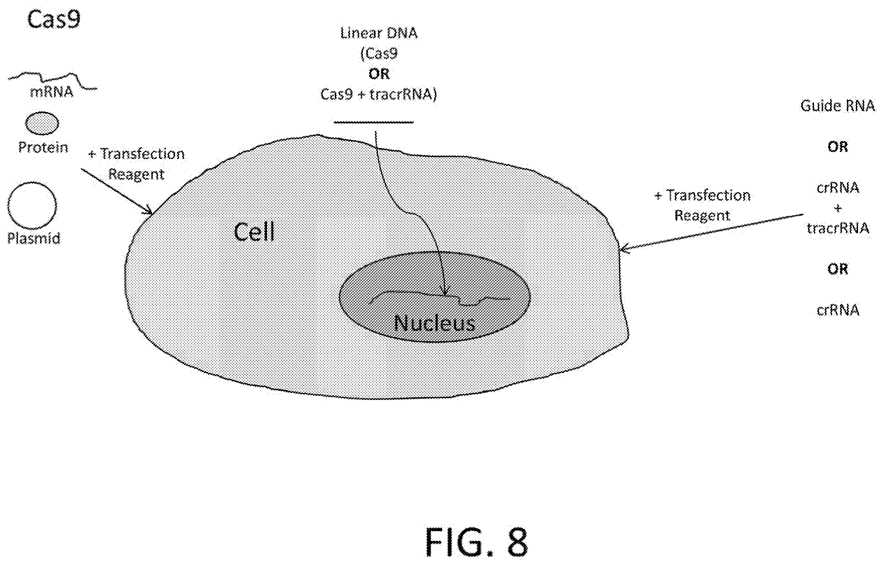

Transient activity can be achieved in a number of ways, some of which are represented in FIG. 8. CRISPR systems typically require that all necessary components be present for activity. Using a CRISPR/Cas9 system for reference, a target nucleic acid molecule must be contacted with a Cas9 protein and one or more CRISPR nucleic acid molecules (e.g., either (1) a crRNA molecule and a tracrRNA molecule or (2) a guide RNA molecule).

The invention thus includes compositions and methods for transient CRISPR mediate activities (e.g., nuclease activity). Transient activity may be the generated in any number of ways. One feature of CRISPR systems is that all components typically need to come together for activity. These components are (1) one or more CRISPR proteins (e.g., Cas9), (2) Hybridization Region 1 (e.g., crRNA), and (3) nucleic acid that associates with both Hybridization Region 1 and the one or more CRISPR proteins. Thus, if one or more components required for CRISPR mediate activity is removed, then the activity is inhibited.

Using the Cas9 based CRISPR system for purposes of illustration, three components are required for CRISP mediated activity: (1) Cas9 protein, (2) crRNA, and (3) tracrRNA. Thus, transient systems can be generated by the time limited presence of any one of these components. A number of variations are represented in FIG. 8.

TABLE 3
Exemplary CRISPR Components

| | | Format 1 | | Format 2 |
|---|---|---|---|---|
| Row | Cas9 Protein | crRNA | tracrRNA | Guide RNA |
| No. | (Col. A) | (Col. B) | (Col. C) | (Col. D) |
| 1 | Integrated | Integrated | Integrated | Integrated |
| | Coding | Coding | Coding | Coding |
| | Seq. | Seq. | Seq. | Seq. |
| 2 | Protein | crRNA | tracrRNA | Guide RNA |
| 3 | Linear | Linear | Linear | Linear |
| | Coding Seq. | Coding Seq. | Coding Seq. | Coding Seq. |
| 4 | Vector | Vector | Vector | Vector |
| 5 | mRNA | — | — | — |

As noted above, in Cas9 mediated system, Cas9 protein must be present for activity. Further, proteins normally are fairly stable molecules within cells. Cas9 proteins may be modified to enhance intracellular degradation (e.g., proteosome mediated degradation) by, for example, ubiquitination.

Cas9 protein may be either introduced into cells (Row 2, Column A) or produced intracellularly (Rows 1, 3, 4, and 5, Column A). Further, the duration of time that Cas9 protein is taken up or produced intracellularly and the amount that is present intracellularly may be controlled or regulated. As an example, a chromosomally integrated Cas9 protein coding sequence may be operably linked to a regulatable promoter. Further, the amount of mRNA encoding Cas9 protein introduced into cells may be regulated.

With respect to non-coding CRISPR RNA needed to high level CRISPR activity, at least two formats are possible: (1) separate crRNA and tracrRNA molecules and (2) Guide RNA (see Table 3).

The invention thus includes compositions and method for transient production of CRISPR mediated activities within cells. Such methods include, for example, the use of a combination of stable and unstable CRISPR system components. One example is a system where mRNA encoding wild-type Cas9 protein and a guide RNA are introduced into a cell in roughly equal amounts. In this example, the presence of Cas9 mRNA will result in the production of a stable Cas9 protein and the limiting factor on CRISPR mediated activity will typically be the determined by the amount of guide RNA present and guide RNA degradation.

The production and/or intracellular introduction of various components of CRISPR mediated systems in a number of ways. For example, a cell designed for convenient CRISPR system reconstitution could be produced. One example of such a cell would be a mammalian cell line (e.g., CHO, 293, etc.) that contains nucleic acid encoding Cas9 protein and tracrRNA integrated into the genome. CRISPR mediated activities can then be directed to a specific target sequence by the introduction into the cell line (e.g., via transfection) of crRNA. In such an exemplary cell line, Cas9 and/or tracrRNA coding sequences may be constitutively expressed or regulatably expressed (e.g., operably linked to an inducible or a repressible promoter).

The invention thus includes cell lines (e.g., eukaryotic cells lines) that contain one or more component of a CRISPR system, as well as methods for directing one or more CRISPR mediated activity to specific target loci within such cells. In many instances, this will result from the addition to or production of at least one additional component that results in target locus CRISPR mediated activities within the cell.

### CRISPR Proteins

Depending upon the type of CRISPR system, one or more CRISPR proteins (e.g., Cas9) may be used. These CRISPR proteins are targeted to a first nucleic acid of defined sequence (a target locus) by a second nucleic acid and function either alone or in conjunction with other proteins. Thus, the CRISPR complex is a nucleic acid guided, nucleic acid recognition system.

CRISPR proteins or protein complexes will typically have binding activity for one or more CRISPR oligonucleotides and a nucleic acid modification activity (e.g., recombinase activity, methylase activity, etc.). Further, a nuclear localization signal may be present in CRISPR proteins or protein complexes, especially when (1) generated in or (2) designed or produced for introduction into a eukaryotic cell.

Thus, CRISPR proteins may be fusion proteins comprising, for example, the CRISPR protein or fragment thereof and an effector domain. Suitable effector domains include, for example nucleic acid cleavage domains (e.g., heterologous cleavage domains such as the cleavage domain of the endonuclease FokI), epigenetic modification domains, transcriptional activation domains (e.g., a VP16 domain), and transcriptional repressor domains. Each fusion protein may be guided to a specific chromosomal locus, for example, by a specific guide RNA, wherein the effector domain mediates targeted genome modification or gene regulation.

In some aspects, the fusion proteins can function as dimers thereby increasing the length of the target site and increasing the likelihood of its uniqueness in the genome (thus, reducing off target effects). For example, endogenous CRISPR systems modify genomic locations based on DNA binding word lengths of approximately 13-20 bp (Cong et al., Science, 339:819-823 (2013).

CRISPR proteins may be synthesized and/or purified by any number of means. In many instances, CRISPR proteins will be produced within the cell in which activity is desired. In some instances, CRISPR proteins may be produced extracellular to the cell in which activity is desired and then introducing into the cell. Example of methods for producing such CRISPR proteins is by in vitro translation, extraction of the proteins from cell that express these proteins encoded by an expression vector, and extraction of these proteins from cell that normally express them.

### CRISPR Oligonucleotides

CRISPR oligonucleotides may be produced by a number of methods and may be generated to have varying features. In many instances, CRISPR oligonucleotides will be one component or two components. By “one component” is meant that only one oligonucleotide (e.g., guide RNA) is necessary for CRISPR activity. By “two components” is meant that only two different oligonucleotides (e.g., crRNA and tracrRNA) are required for CRISPR activity. CRISPR systems with more than two components may also be designed, produced and used. Thus, the invention contemplates multi-components CRISPR oligonucleotides where functionality involves three, four, five, etc. oligonucleotides.

In some instances, two or more oligonucleotides may be generated separately and then joined to each other to form, for example, one oligonucleotide that functions as part of a CRISPR system. The number of components of a system is determined by interaction with Cas9. As an example, if two oligonucleotides are produced and then joined prior to introduction into a cell, where the joined oligonucleotide requires no additional oligonucleotides to facilitate a CRISPR mediated activity, then this is said to be a one component system.

Of course, the nucleotide sequences and other features of CRISPR oligonucleotides may vary with specific systems and desired functions. Common features of CRISPR oligonucleotides include association with one or more CRISPR complex protein (e.g., Cas9) and nucleic acid “targeting” capability.

The invention thus includes compositions and methods for the production of CRISPR oligonucleotides, as well as collections of oligonucleotides generated, for example, using such compositions and methods.

In some embodiments, compositions and methods of the invention are directed to one of or a combination of molecular biology synthesis (e.g., PCR) and/or chemical synthesis for the generation of CRISPR oligonucleotides. Using the schematic representation shown in FIG. 6 for reference, two chemically synthesized oligonucleotides encoding components of a guide RNA may be designed to hybridize with each other and be extended to form a fully double-stranded nucleic acid molecule (e.g., DNA).

FIG. 9 shows an exemplary workflow of the invention. The schematic in FIG. 9 shows oligonucleotides designed to generate a DNA molecule where the guide RNA coding region is operably linked to a T7 promoter. In this work flow DNA oligonucleotides either alone or in conjunction with double-stranded DNA are used to generate, via PCR, a DNA molecule encoding a guide RNA operably linked to a promoter suitable for in vitro transcription. The DNA molecule is then transcribed in vitro to generate guide RNA. The guide RNA may then be “cleaned up” by, for example, column purification or bead based methods. The guide RNA is then suitable for use by, as examples, (1) direct introduction into a cell or (2) introduction into a cell after being complexed with one or more CRISPR protein. Nucleic acid operably connected to a T7 promoter can be transcribed in mammalian cells when these cells contain T7 RNA polymerase (Lieber et al., Nucleic Acids Res., 17: 8485-8493 (1989)). Of course, other promoters functional in eukaryotic cells (e.g., CMV promoter, U6 promoter, H1 promoter, etc.) could also be used for the intracellular production of guide RNA. The H1 promoter, for example, is about 300 base pairs in length. One advantage of the T7 promoter is its small size (20 base pairs). On specific T7 promoter that may be used in compositions and methods of the invention include those having the following nucleotide sequence: GAAATTAATACGACTCACTATAG (SEQ ID NO: _).

The T7 promoter may also be used to generate guide RNA in an in vitro transcription system. In this instance, the double-stranded nucleic acid molecule would be used to generate guide RNA extracellularly for introduction into a cell.

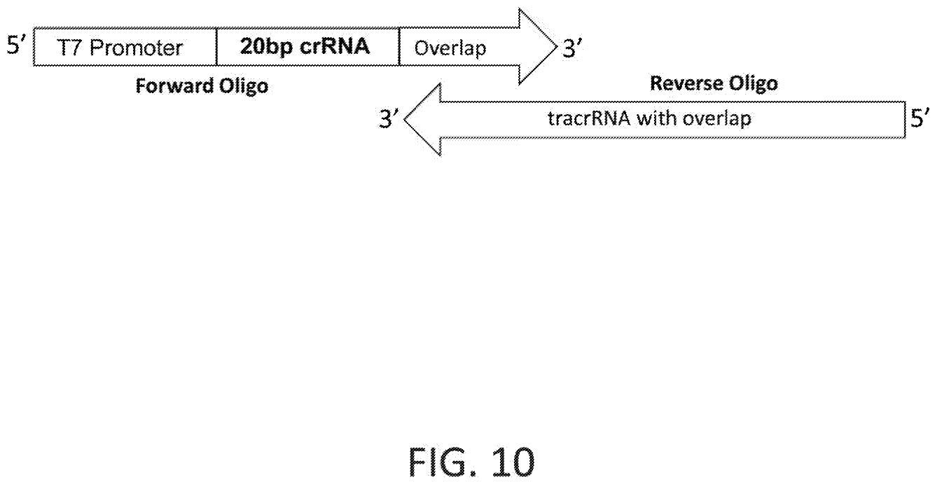

Advantages of the guide RNA generation methods set out, for example, in FIGS. 9 and 10 are speed and low cost of production. In particular, once a target sequence has been identified the Forward Oligo may be generated and combined with the Reverse Oligo in a reaction mixture designed to extend each of the oligonucleotides to form the double-stranded nucleic acid molecule (see FIG. 10). The Forward Oligo encodes the crRNA sequence designed with sequence complementarity to the target locus. Further, the Reverse Oligo has a sequence that is common to guide RNAs. The Reverse Oligo may be generated by any means and stored as a standard component. The Forward Oligo, however, is target sequence specific so it must be designed in view of the target locus.

Two oligonucleotides suitable for the generation of double-stranded DNA suitable for transcription as set out in FIG. 9 are shown in FIG. 10. In the schematic of FIG. 10, the “Forward Oligo” is tailored for the target locus because it contains Hybridization Region 1 of the target specific crRNA. The “Reverse Oligo” contains regions of the tracrRNA and crRNA that are not target locus specific. Thus, the “Reverse Oligo” can be a “stock” component. The invention thus includes compositions and methods for the formation of guide RNA molecules. Methods of this aspect of the invention may comprise one or more of the following,

a. identification of a target locus,
b. the in silico design of one or more CRISPR RNA molecules with sequence complementarity to that locus,
c. the production of a first oligonucleotide with a promoter sequence, a region of sequence complementarity (e.g., 15 to 25 nucleotides in length) to the target locus,
d. incubating the first oligonucleotide with (i) a second oligonucleotide and (ii) a polymerase under conditions suitable for performing polymerase chain reaction (PCR) to generate a double-stranded nucleic acid molecule, wherein the first oligonucleotide and the second oligonucleotide have a region of sequence complementarity of sufficient length to allow for hybridization between the two oligonucleotides, and
e. performing an in vitro transcription reaction on the PCR generated double-stranded nucleic acid molecule to produce a guide RNA molecule, and
f. purifying the guide RNA molecule from the other components of the reaction mixture.

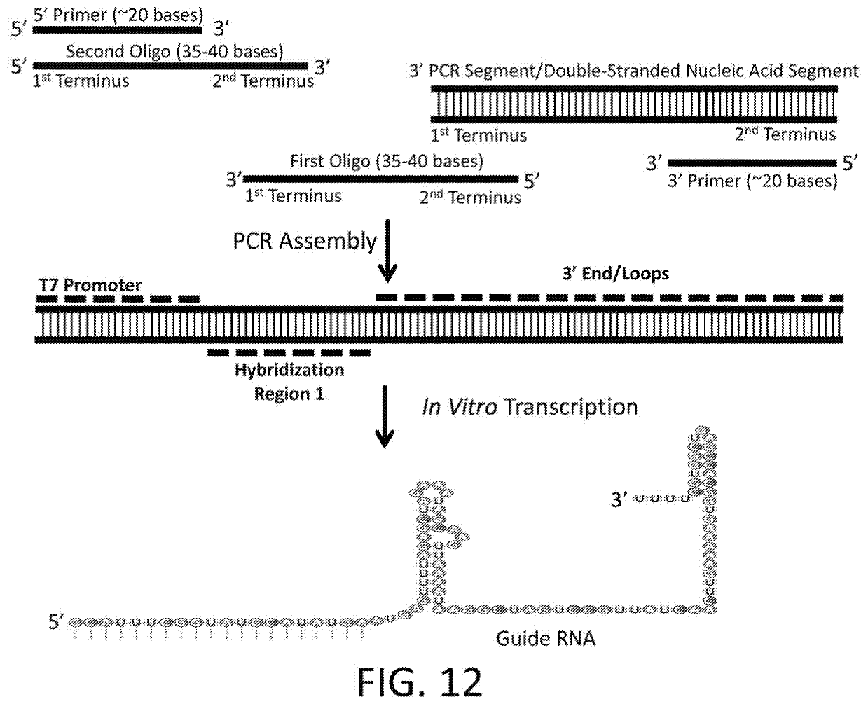

In the work flow shown in FIG. 12, two oligonucleotides with the T7 promoter and Hybridization Region 1 nucleic acid and a Double-Stranded Nucleic Acid Segment are assembled by PCR. In this workflow, the Double-Stranded Nucleic Acid Segment has a constant sequence and, thus, can be a stock component.

The two oligonucleotides form the full length double-stranded nucleic acid segment via a polymerase mediated assembly reaction. Once the full length product molecule is assembled, further PCR reactions amplify the product. The primers prevent the two oligonucleotides from being PCR “limiting” components. In other words, once the product nucleic acid molecule has been generated, the primers allow for amplification to continue after the first and second oligonucleotides have been consumed.

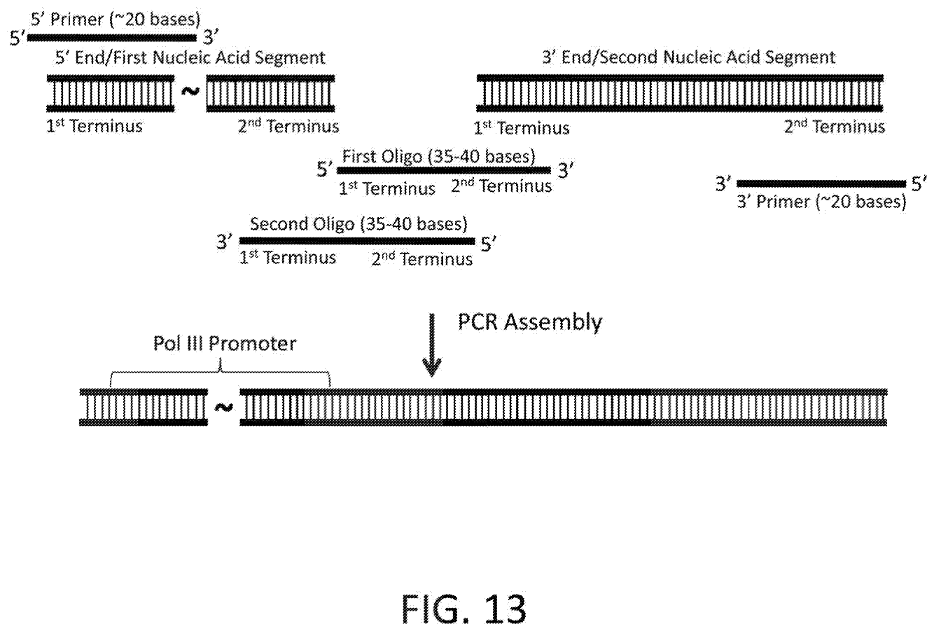

FIG. 13 shows a process similar to that represented in FIG. 12 but the assembly reaction links two double-stranded nucleic acid segments and inserts specific nucleic acid in between them. Thus, the method represented in FIG. 10 is especially useful for the insertion of a nucleic acid segment of designed sequence between to selected nucleic acid molecules.

With respect to CRISPR RNA coding sequence construction, the First Oligonucleotide and the Second Oligonucleotide may be synthesized to hybridize with the First Nucleic Acid Segment and the Second Nucleic Acid Segment. Each of these oligonucleotides also encode all or part of Hybridization Region 1. Assembly reactions may thus be designed to generate, for example, a DNA molecule that encodes a target locus specific guide RNA operably linked to a promoter.

While only one oligonucleotide is required for assembly reactions of the type shown in FIG. 13, two will generally be used because crRNA Hybridization Region 1 is typically about 20 bases in length and about 15 bases of sequence identity is desired for efficient hybridization to the First Nucleic Acid Segment and the Second Nucleic Acid Segment. While oligonucleotides of 45 to 55 bases can be chemically synthesized, sequence fidelity often drops with length. The introduction of crRNA Hybridization Region 1 segment with low sequence results in two issues: (1) An increase in “off-target” effects can occur due to the Hybridization Region 1 associating with loci other that the desired target locus and (2) decreased target locus interaction efficiency of the encoded guide RNAs.

The second issue above occurs when heterogeneous PCR assembled nucleic acid (e.g., DNA) are transcribed (e.g., via in vitro transcription) and then introduced into cells. In general, the lower the level of sequence fidelity in the original assembly oligonucleotide population, the greater the variation in Hybridization Region 1 of the expressed guide RNA population. One way to address this problem is to use oligonucleotides generated with high sequence fidelity.

FIG. 13 represents a design for synthetic guide RNA expression cassette assembly. In this design, target specific variable crRNA region is encoded by the 35 to 40 base pair DNA oligo (represented here has first and second oligo). All the remaining DNA oligos and double stranded DNA segments may be constant components. These constant components include 1) a first double stranded nucleic acid segment encoding, for example, an RNA polymerase III promoter that can be leveraged for expressing the non-coding guide RNA component in vivo 2) a second double stranded nucleic acid segment encoding the tracrRNA component, and 3) 5′ and 3′ primers for amplification and enrichment of full length guide RNA expression templates containing the RNA polymerase III promoter. Full length guide RNA expression cassette containing relevant RNA polymerase III promoter is generated by assembly PCR using the double stranded nucleic acid segments, target specific overlapping oligos and flanking PCR primers. Assembly PCR is performed using a Taq DNA polymerase (e.g., PHUSION® Taq DNA polymerase), with the resulting product being column purified prior to delivery into host cell line of interest. Methods such as this can also be used to generate guide RNA expression cassettes containing any user defined promoter.

The invention further includes compositions and methods for the assembly of CRISPR RNA molecules (e.g., guide RNA molecules). CRISPR RNA molecules may be assembled by the connection of two or more (e.g., two, three, four, five, etc.) RNA segments with each other. In particular, the invention includes methods for producing nucleic acid molecules, these methods comprising contacting two or more linear RNA segments with each other under conditions that allow for the 5′ terminus of a first RNA segment to be covalently linked with the 3′ terminus of a second RNA segment.

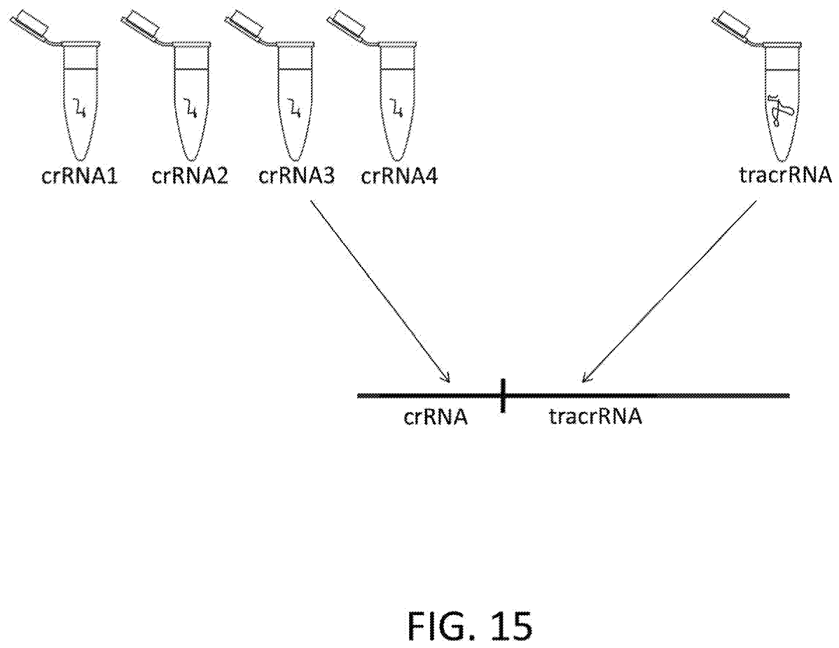

This form of assembly has the advantage that it allows for rapid and efficient assembly of CRISPR RNA molecules. Using the schematic shown in FIG. 15 for purposes of illustration, guide RNA molecules with specificity for different target sites can be generated using a single tracrRNA molecule/segment connected to a target site specific crRNA molecule/segment. FIG. 15 shows four tubes with different crRNA molecules with crRNA molecule 3 being connected to a tracrRNA molecule to form a guide RNA molecule. Thus, FIG. 15 shows the connection of two RNA segments to for a product RNA molecule. Thus, the invention includes compositions and methods for the connection (e.g., covalent connection) of crRNA molecules and tracrRNA molecules.

The invention also includes compositions and methods for the production of guide RNA molecules with specificity for a target site, the method comprising: (1) identification of the target site, (2) production of a crRNA segment, and (3) connection of the crRNA segment with a tracrRNA segment. In such methods, the tracrRNA segment may be produced prior to connection with the crRNA and stored as a “stock” component or the tracrRNA segment may be generated from a DNA molecule that encodes the tracrRNA.

RNA molecules/segments connected to each other in the practice of the invention may be produced by any number of means, including chemical synthesis and transcription of DNA molecules. In some instances, RNA segments connected to each other may be produced by different methods. For example, a crRNA molecule produced by chemical synthesis may be connected to a tracrRNA molecule produced by in vitro transcription of DNA or RNA encoding the tracrRNA.

RNA segments may also be connected to each other by covalent coupling. RNA ligase, such as T4 RNA ligase, may be used to connect two or more RNA segments to each other. When a reagent such as an RNA ligase is used, a 5′ terminus is typically linked to a 3′ terminus. If two segments are connected, then there are two possible linear constructs that can be formed (i.e., (1) 5′-Segment 1-Segment 2-3′ and (2) 5′-Segment 2-Segment 1-3′). Further, intramolecular circularization can also occur. Both of these issues can be addressed by blocking one 5′ terminus or one 3′ terminus so that RNA ligase cannot ligate the terminus to another terminus. Thus, if a construct of 5′-Segment 1-Segment 2-3′ is desired, then placing a blocking group on either the 5′ end of Segment 1 or the 3′ end of Segment 2 will result in the formation of only the correct linear ligation product and will prevent intramolecular circularization. The invention thus includes compositions and methods for the covalent connection of two nucleic acid (e.g., RNA) segments. Methods of the invention include the use of an RNA ligase to directionally ligate two single-stranded RNA segments to each other.

One example of an end blocker that may be used in conjunction with, for example, T4 RNA ligase is a dideoxy terminator.

T4 RNA ligase catalyzes the ATP-dependent ligation of phosphodiester bonds between 5′-phosphate and 3′-hydroxyl termini. Thus, when one uses T4 RNA ligase, suitable termini must be present on the termini being ligated. One means for blocking T4 RNA ligase on a terminus is by failing to have the correct terminus format. In other words, termini of RNA segments with a 5-hydroxyl or a 3′-phosphate will not act as substrates for T4 RNA ligase.

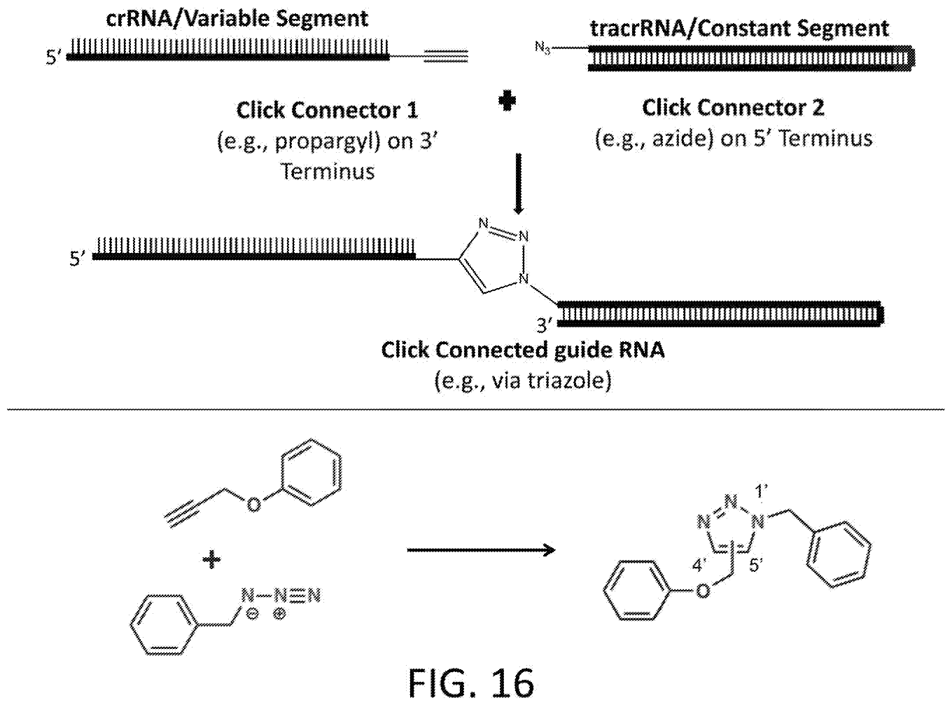

Another method that may be used to connect RNA segments is by “click chemistry” (see, e.g., U.S. Pat. Nos. 7,375,234 and 7,070,941, and US Patent Publication No. 2013/0046084, the entire disclosures of which are incorporated herein by reference). For example, one click chemistry reaction is between an alkyne group and an azide group (see FIG. 16). Any click reaction can be used to link RNA segments (e.g., Cu-azide-alkyne, strain-promoted-azide-alkyne, staudinger ligation, tetrazine ligation, photo-induced tetrazole-alkene, thiol-ene, NHS esters, epoxides, isocyanates, and aldehyde-aminooxy). Ligation of RNA molecules using a click chemistry reaction is advantageous because click chemistry reactions are fast, modular, efficient, often do not produce toxic waste products, can be done with water as a solvent, and can be set up to be stereospecific.

In one embodiment the present invention uses the “Azide-Alkyne Huisgen Cycloaddition” reaction, which is a 1,3-dipolar cycloaddition between an azide and a terminal or internal alkyne to give a 1,2,3-triazole for the ligation of RNA segments. One advantage of this ligation method is that this reaction can initiated by the addition of required Cu(I) ions.

Other mechanism by which RNA segments may be connected include the use of halogens (F-, Br-, I-)/alkynes addition reactions, carbonyls/sulfhydryls/maleimide, and carboxyl/amine linkages.

For example, one RNA molecule may be modified with thiol at 3′ (using disulfide amidite and universal support or disulfide modified support), and the other RNA molecule may be modified with acrydite at 5′ (using acrylic phosphoramidite), then the two RNA molecules can be connected by Michael addition reaction. This strategy can also be applied to connecting multiple RAN molecules stepwise.

The invention also includes methods for linking more than two (e.g., three, four, five, six, etc.) RNA molecules to each other. One reason this may be done is when an RNA molecule longer than about 40 nucleotides is desired, as noted elsewhere herein, chemical synthesis efficiency degrades.

By way of example, a tracrRNA is typically around 80 nucleotides. Such RNA molecules may be produced by processes such as in vitro transcription or chemical synthesis. When chemical synthesis is used to produce such RNA molecules, they may be produced as a single synthesis product or by linking two or more synthesized RNA segments to each other. Further, when three or more RNA segments are connected to each other, different methods may be used to link the individual segments together. Also, the RNA segments may be connected to each other in one “pot”, all at the same time, or in one “pot” at different times or in different “pots” at different times.

For purposes of illustration, assume one wishes to assemble RNA Segments 1, 2 and 3 in numerical order. RNA Segments 1 and 2 may be connected, 5′ to 3′, to each other. The reaction product may then be purified for reaction mixture components (e.g., by chromatography), then placed in a second vessel, “pot”, for connection of the 3′ terminus with the 5′ terminus of RNA Segment 3. The final reaction product may then be connected to the 5′ terminus of RNA Segment 3.

A second, more specific illustration of one embodiment of the invention is as follows. RNA Segment 1 (about 30 nucleotides) is the target locus recognition sequence of a crRNA and a portion of Hairpin Region 1. RNA Segment 2 (about 35 nucleotides) contains the remainder of Hairpin Region 1 and some of the linear tracrRNA between Hairpin Region 1 and Hairpin Region 2. RNA Segment 3 (about 35 nucleotides) contains the remainder of the linear tracrRNA between Hairpin Region 1 and Hairpin Region 2 and all of Hairpin Region 2. In this illustration, RNA Segments 2 and 3 are linked, 5′ to 3′, using click chemistry. Further, the 5′ and 3′ end termini of the reaction product are both phosphorylated. The reaction product is then contacted with RNA Segment 1, having a 3′ terminal hydroxyl group, and T4 RNA ligase to produce a guide RNA molecule.

A number of additional linking chemistries may be used to connect RNA segments according to method of the invention. Some of these chemistries are set out in Table 4.

TABLE 4
Exemplary RNA Ligation Reactions

| Reaction Type | Reaction Summary |
|---|---|
| Thiol-yne | 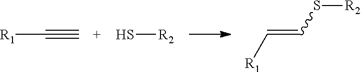 |
| NHS esters | 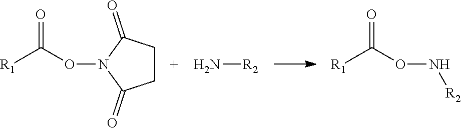 |
| Thiol-ene | 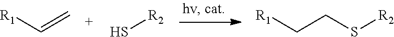 |
| Isocyanates | 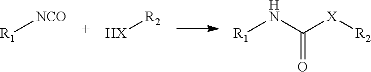 X = S or NH |
| Epoxy or aziridine | 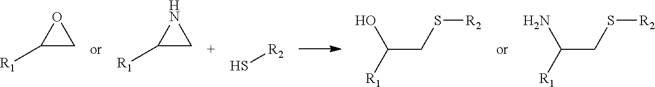 |
| Aldehyde- aminoxy | 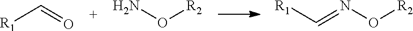 |
| Cu-catalyzed- azid-alkyne | 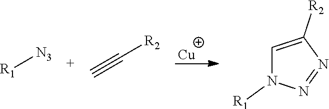 |
| Strain- promoted-azid- alkyne | Cyclooctyne cycloaddition (with azide or nitrile oxide or nitrone) 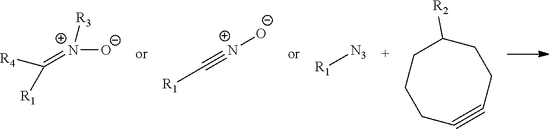 |
| | 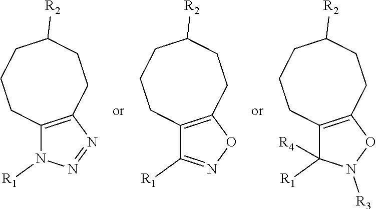 |
| | Norbornene cycloaddition (with azide or nitrile oxide or nitrone) 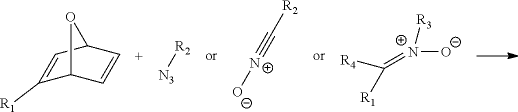 |
| | 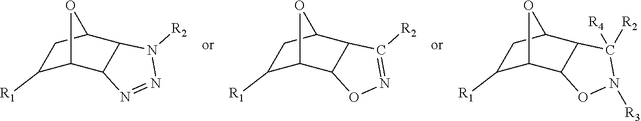 |
| | Oxanorbornadiene cycloaddition 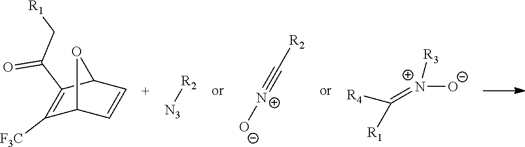 |
| | 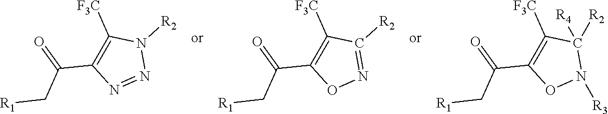 |
| Staudinger ligation | 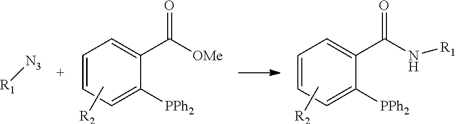 |
| Tetrazine ligation | 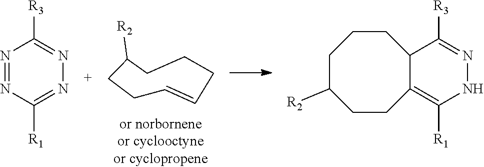 |
| Photo-induced tetrazole-alken | 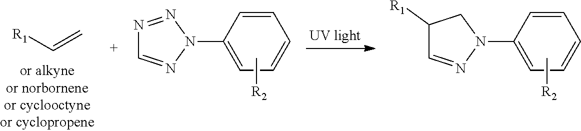 |
| [4 + 1] cycloaddition | 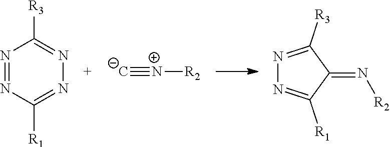 |
| Quadricyclane ligation | 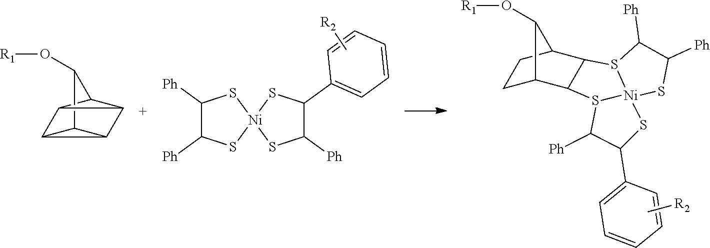 |

One issue with methods for linking RNA segments is that often they do not result in complete conversion of the segments to connected RNA molecules. For example, some chemical linkage reactions only result in 50% of the reactants forming the desired end product. In such instances, it will often be desirable to remove reagents and unreacted RNA segments. This may be done by any number of means such as dialysis, chromatography (e.g., HPLC), precipitation, electrophoresis, etc. Thus, the invention includes compositions and method for linking RNA segments, where the reaction products RNA molecules are separated from other reaction mixture components.

As noted above, CRISPR system components may be “generic” with respect to target loci (e.g., Cas9 protein) or may be specific for a particular target locus (e.g., crRNA). This allows for the production of “generic” components that may be used in conjunction with target sequence specific components. Thus, when a target locus of interest is identified, one need only produce a component or components specific for that target locus. In the instance where one seeks to make two closely associated “nicks” at the target sequence, then, for example, two crRNA molecules will typically need to be produced. These crRNA molecules may be produced when the target sequence of interest is identified or they may be produced in advance and stored until needed.

The invention further includes collections of crRNA molecules with specificity for individual target sites. For example, the invention includes collections of rRNA molecules with specificity for target sites within particular types of cell (e.g., human cells). The members of such collection of cells may be generated based upon sequence information for these particular types of cells. As an example, one such collection could be generated using the complete genome sequence of a particular type of cell. The genome sequence data can be used to generate a library of crRNA molecules with specificity for the coding region of each gene within the human genome. Parameters that could be used to generate such a library may include the location of protospacer adjacent motif (PAM) sites, off target effects (e.g., sequences unique to the target region), and, when gene “knockouts” are desired, locations within coding regions likely to render the gene expression product fully or partially non-functional (e.g., active site coding regions, intron/exon junctions, etc.).

Collections or libraries of crRNA molecules or the invention may include a wide variety of individual molecules such as from about five to about 100,000 (e.g., from about 50 to about 100,000, from about 200 to about 100,000, from about 500 to about 100,000, from about 800 to about 100,000, from about 1,000 to about 100,000, from about 2,000 to about 100,000, from about 4,000 to about 100,000, from about 5,000 to about 100,000, from about 50 to about 50,000, from about 100 to about 50,000, from about 500 to about 50,000, from about 1,000 to about 50,000, from about 2,000 to about 50,000, from about 4,000 to about 50,000, from about 50 to about 10,000, from about 100 to about 10,000, from about 200 to about 10,000, from about 500 to about 10,000, from about 1,000 to about 10,000, from about 2,000 to about 10,000, from about 4,000 to about 10,000, from about 50 to about 5,000, from about 100 to about 5,000, from about 500 to about 5,000, from about 1,000 to about 5,000, from about 50 to about 2,000, from about 100 to about 2,000, from about 500 to about 2,000, etc.).

RNA molecules generated by and used in the practice of the invention may be stored in a number of ways. RNA molecules are generally not as stable as DNA molecules and, thus, to enhance stability, RNA molecules may be stored at low temperature (e.g., −70° C.) and/or in the presence of one or more RNase inhibitor (e.g., RNASEOUT™, RNASECURE™ Reagent, both available from Thermo Fisher Scientific).

Further, RNA molecules may be chemically modified to be resistant to RNases by, for example, being generated using RNase-resistant ribonucleoside triphosphates. Examples of RNase-resistant modified ribonucleosides include, but are not limited to, 2-fluoro ribonucleosides, 2-amino ribonucleosides, and 2-methoxy ribonucleosides. Additional examples of RNase-resistant modified ribonucleosides are disclosed in U.S. Patent Publ. 2014/0235505 A1, the entire disclosure of which is incorporated herein by reference. 2′-O-allyl-ribonucleotides may also be incorporated into RNA molecules of the invention.

Chemical modification used in the practice of the invention will often be selected based upon a series of criteria, such as effectiveness for the purpose that the chemical modification is used (e.g., RNase resistance), level of toxicity to cells (low generally being better than high), ease of incorporation into the nucleic acid molecules, and minimal interference with the biological activities of the nucleic acid molecule (e.g., the activities of a guide RNA molecule).

Further, RNA molecules of and used in the practice of the invention may be stored in a number of different formats. For example, RNA molecules may be stored in tubes (e.g., 1.5 ml microcentrifuge tubes) or in the wells of plates (e.g., 96 well, 384 well, or 1536 well plates).

The invention thus includes compositions and methods for the production of libraries and/or collections of CRISPR system components, as well as the libraries and/or collections of CRISPR system components themselves.

The invention also includes compositions and methods for the isolation of gRNA molecules. Such methods will often be based upon hybridization of a gRNA region to another nucleic acid molecule, followed by separation of the hybridized complex from other molecules (e.g., nucleic acid molecules) present in a mixture.

As an example, beads containing a nucleic acid molecule with sequence homology to a gRNA molecule may be used to purify the gRNA from a solution. In some instances, the bead will be a magnetic bead. Further, the nucleic acid molecule designed to hybridize to the gRNA molecule may be designed with homology to a sequence present in gRNA molecules or gRNA molecules may be designed to contain a sequence that is used for hybridization. The invention thus includes gRNA molecules that are designed to contain what is effectively a hybridization “tag”.

Such “tags” are particularly useful in high throughput applications. As an example, a 96 well plate may contain different gRNA molecules in each well, wherein each gRNA molecules contains the same tag. A magnetic bead may be placed in one or more well of the plate and then removed after a specified period of time to allow for gRNA/bead bound hybridization to take place. These beads may then be individually placed in wells of another plate containing cells and donor DNA under conditions that allow for release of gRNA molecules from the beads (e.g., competition with an oligonucleotide of identical or similar sequence to the tag).

As noted above, hybridization tags may be naturally resident with gRNA molecules or may be introduced into or added to gRNA molecules. Such tags may be added by the alteration of a region present in a gRNA molecule or may be added to the gRNA either internally or at a terminus. Further, tags may be generated during synthesis of gRNA molecules or added after gRNA molecules are produced (e.g., via “click chemistry”).

Hybridization tags will typically be less than 25 (e.g., from about 10 to about 25, from about 15 to about 25, from about 16 to about 25, from about 10 to about 20, from about 15 to about 25, from about 15 to about 20, etc.) bases in length. Such tags will typically be able to hybridize to homologous sequences with sufficient affinity for association but will not associate so strongly that they do not efficiently release when desired. Further, shorter tags will often have a higher GC content. In many instances, tags will have a GC content of at least 45% (e.g., from about 45% to about 75%, from about 50% to about 75%, from about 55% to about 75%, from about 60% to about 75%, from about 65% to about 75%, etc.).

Also, tagged gRNA molecules may contain a label. This label may be used to quantify the amount of gRNA present. Labels may also be useful when seeking to determine the amount of gRNA transferred by hybridization based means. Such labels may also be used to measure cellular uptake as set out elsewhere herein.

CRISPR complexes of the invention can have any number of activities. For example, CRISPR proteins may be fusion proteins comprising one or more heterologous protein domains (e.g., one, two, three, four, five, etc.). A CRISPR fusion protein may comprise any additional protein sequence, and optionally a linker sequence between any two domains. Examples of protein domains that may be fused to a CRISPR protein include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity, and nucleic acid binding activity.

Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

A CRISPR protein may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GALA DNA binding domain fusions, and herpes simplex virus (HSV) BP16 protein fusions. Additional domains that may form part of a fusion protein comprising a CRISPR protein are described in US 2011/0059502, incorporated herein by reference.

In particular, provided herein, in part, are CRISPR protein endonucleases, which comprise at least one nuclear localization signal, at least one nuclease domain, and at least one domain that interacts with a guide RNA to target the endonuclease to a specific nucleotide sequence for cleavage. Also provided are nucleic acids encoding CRISPR protein endonucleases, as well as methods of using CRISPR protein endonucleases to modify chromosomal sequences of eukaryotic cells or embryos. CRISPR protein endonucleases interacts with specific guide RNAs, each of which directs the endonuclease to a specific targeted site, at which site the CRISPR protein endonucleases introduces a double-stranded break that can be repaired by a DNA repair process such that the chromosomal sequence is modified. Since the specificity is provided by the guide RNA (or the crRNA), the CRISPR protein endonucleases are universal and can be used with different guide RNAs to target different genomic sequences. Methods disclosed herein can be used to target and modify specific chromosomal sequences and/or introduce exogenous sequences at targeted locations in the genome of cells or embryos.

CRISPR complexes may also be employed to activate or repress transcription. For example, a dCas9-transcriptional activator fusion protein (e.g., dCas9-VP64) may be used in conjunction with a guide RNA to activate transcription of nucleic acid associated with a target locus. Similarly, dCas9-repressor fusions (e.g., dCas9-KRAB transcriptional repressor) may be used to repress transcription of nucleic acid associated with a target locus. Transcriptional activation and repression such as the referred to above are discussed in, for example, Kearns et al., Cas9 effector-mediated regulation of transcription and differentiation in human pluripotent stem cells, Development, 141:219-223 (2014).

The invention thus includes compositions and methods for the production and use of CRISPR system components for the activation and repression of transcription.

Transfection agents suitable for use with the invention include transfection agents that facilitate the introduction of RNA, DNA and proteins into cells. Exemplary transfection reagents include TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LIPOFECTAMINE™ 2000, LIPOFECTAMINE™ 3000 (Thermo Fisher Scientific), LIPOFECTAMINE™ (Thermo Fisher Scientific), LIPOFECTIN™ (Thermo Fisher Scientific), DMRIE-C, CELLFECTIN™ (Thermo Fisher Scientific), OLIGOFECTAMINE™ (Thermo Fisher Scientific), LIPOFECTACE™, FUGENE™ (Roche, Basel, Switzerland), FUGENE™ HD (Roche), TRANSFECTAM™ (Transfectam, Promega, Madison, Wis.), TFX-10™ (Promega), TFX-20™ (Promega), TFX-50™ (Promega), TRANSFECTIN™ (BioRad, Hercules, Calif.), SILENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT4™ (Dharmacon), ESCORT™ III (Sigma, St. Louis, Mo.), and ESCORT™ IV (Sigma Chemical Co.).

The invention further includes methods in which one molecule is introduced into a cell, followed by the introduction of another molecule into the cell. Thus, more than one CRISPR system components molecule may be introduced into a cell at the same time or at different times. As an example, the invention includes methods in which Cas9 is introduced into a cell while the cell is in contact with a transfection reagent designed to facilitate the introduction of proteins in to cells (e.g., TurboFect Transfection Reagent), followed by washing of the cells and then introduction of guide RNA while the cell is in contact with LIPOFECTAMINE™ 2000.

Conditions will normally be adjusted on, for example, a per cell type basis for a desired level of CRISPR system component introduction into the cells. While enhanced conditions will vary, enhancement can be measure by detection of intracellular CRISPR system activity. Thus, the invention includes compositions and methods for measurement of the intracellular introduction of CRISPR system components in cells.

The invention also includes compositions and methods related to the formation and introduction of CRISPR complexes into cells. One exemplary method of the invention comprises:

a. forming a complex comprising at least one CRISPR system protein with at least one CRISPR RNA,
b. contacting the complexed CRISPR system protein and RNA with a cell,
c. incubating or culturing the resulting cell for a period of time (e.g., from about 2 minutes to about 8 hours, from about 10 minutes to about 8 hours, from about 20 minutes to about 8 hours, from about 30 minutes to about 8 hours, from about 60 minutes to about 8 hours, from about 20 minutes to about 6 hours, from about 20 minutes to about 3 hours, from about 20 minutes to about 2 hours, from about 45 minutes to about 3 hours, etc.), and
d. measuring CRISPR system activity within the cell.

In some instances, during the practice of methods of the invention, molecules introduced into cells may be labeled. As an example, donor DNA may be labeled with ALEXA FLUOR® 647 dye (Thermo Fisher Scientific) and GFP-Cas9 RNP complexes are sequentially introduced into HEK293 cells, followed by cell sorting to obtain cells that contain both labels. This is advantageous because cells containing specific amounts of both labels may be separated from other cells to obtain a population of cells having enhanced probabilities of undergoing genetic modification (e.g., homologous recombination).

Along these lines, the invention also relates to compositions and methods for in situ hybridization, such as fluorescent in situ hybridization (FISH). Nucleic acid molecules introduced into cells for FISH, as well as other purposes may have one or more dephosphorylated terminus, as set out herein. Thus, the invention further relates to compositions and methods for FISH where labeled nucleic acid molecules introduced into cells exhibit low levels of cytotoxicity.

Labels may be attached to one or more CRISPR system component and/or other molecules (e.g., a donor nucleic acid molecule, a FISH probe, etc.) for introduction in the cells. In many instances, labels will be detectable either visually or by cell sorting instruments. Exemplary labels include cyan florescent protein (CFP), green florescent protein (GFP), orange florescent protein (OFP), red florescent protein (RFP), and yellow florescent protein (YFP). Additional labels include AMCA-6-dUTP, DEAC-dUTP, dUTP-ATTO-425, dUTP-XX-ATTO-488, Fluorescein-12-dUTP, Rhodamine-12-dUTP, dUTP-XX-ATTO-532, dUTP-Cy3, dUTP-ATTO-550, dUTP-Texas Red, dUTP-J647, dUTP-Cy5, dUTP-ATTO-647N, dUTP-ATTO-655, Fluorescein-12-dCTP, Rhodamine-12-dCTP, dCTP-Cy3dCTP-ATTO-550, dCTP-Texas Red, dCTP-J647, dCTP-Cy5 and dCTP-ATTO-647N available from multiple sources including Jena Bioscience.

Labels may be located in nucleic acid molecules and proteins at one or both termini and/or interior portions of the particular molecules.

When cells are sorted, a number of separation parameters may be employed. In most instances, sorting may be designed to obtain cells having enhanced probability of undergoing genetic modification. For example, cells may be labeled with different components required for genetic modification followed by cell sorting to obtain cells a specific amount of signal of each component. Further, cells may be selected based upon the number of donor DNA molecules and the number of GFP-Cas9 RNP complexes present within the cells. This may be done by choosing a minimum signal level for each the two labels, resulting in those cells being sorted as “positive” cells. Another sorting option is to score as “positive” cells that are in the top 3%, 5%, 8%, 10%, 15%, 20%, 25%, etc. for both signals as compared to all of the cells in a mixture. Assuming that two labels are equivalently and independently taken up, then scoring for the top 25% of cells for both labels would be expected to yield 6.25% of the original population being sorted. These would likely be the cells in the mixture that have the highest probabilities of undergoing genetic modification.

The invention thus includes methods, as well as compositions for performing such methods, for obtaining cell populations wherein the cells therein have an enhanced probability of undergoing genetic modification. In some instances, such methods will involve one or both of the following: (1) selection of cell (e.g., via cell sorting) of cells that have taken up one or more component necessary for genetic modification (e.g., one or more CRISPR system component and one or more donor DNA molecule) and (2) introduction of one or more one or more component necessary for genetic modification into cells by processes designed to result in high cellular uptake (e.g., sequence component introduction, as set out herein).

Under the particular conditions used, the efficiency of homologous recombination can occur with 0.2 and 0.5 μg of donor DNA per 10 μl reaction volume. The number of cells may also be between 50,000 to 200,000. Further, homologous recombination has been found to decrease with donor DNA levels lower and higher than those amounts. The invention this includes compositions and methods and where donor DNA concentrations are in the range of 0.01 μg/10 μl to 500 μg/10 μl (e.g., from about 0.01 to about 400, from about 0.01 to about 200, from about 0.01 to about 100, from about 0.01 to about 50, from about 0.01 to about 30, from about 0.01 to about 20, from about 0.01 to about 15, from about 0.01 to about 10, from about 0.1 to about 50, from about 0.1 to about 25, from about 0.1 to about 15, from about 0.1 to about 10, from about 0.1 to about 50, from about 0.1 to about 5, from about 0.1 to about 1, from about 0.1 to about 0.8, etc. μg/10 μl.

Also, under the particular conditions used, the efficiency of homologous recombination varies with the length, amount and presence or absence of certain chemical modifications. The length variation may be partially due to the sizes of regions of homology to the locus being modified. For example, about single-stranded donor nucleic acid molecules of about 80 bases in length appear to be sufficient for efficient homologous recombination. In many instances, such donor nucleic molecules will typically have terminal regions of homology to the target locus being modified with a central region containing nucleic acid for insertion at or substitution of nucleic acid at the target locus. In many instances, terminal homologous regions with be between 30 and 50 bases (e.g., from about 30 to about 45, from about 35 to about 45, from about 40 to about 45, from about 40 to about 48, etc.) in length with an intervening region of between 1 and 20 bases (e.g., one, two, three, four five, six, seven, etc.). Further, donor nucleic acid molecules may be used that contain regions of homology designed to hybridize the spatially separated regions of a target locus. In many instances, this spatial separation will be less than about 20 nucleotides. Homologous recombination using such donor nucleic acid molecules would be expected to result in deletion of nucleic acid at the target locus.

The invention further includes compositions and methods for the insertion and correction of single-nucleotide polymorphisms (SNPs). In some instances, such methods involve the use of single-stranded donor nucleic acid with terminal regions having homology to a target site in conjunction with CRISPR system components. Of course, double-stranded donor nucleic acid may also be used for SNP insertion or correction.

A number of compositions and methods may be used to form CRISPR complexes, which may contain one or more dephosphorylated RNA molecule. For example, Cas9 mRNA and a guide RNA may be encapsulated in INVIVOFECTAMINE™ for, for example, later in vivo and in vitro delivery as follows. Cas9 mRNA is mixed (e.g., at a concentration of at 0.6 mg/ml) with guide RNA. The resulting mRNA/gRNA solution may be used as is or after addition of a diluents and then mixed with an equal volume of INVIVOFECTAMINE™ and incubated at 50° C. for 30 min. The mixture is then dialyzed using a 50 kDa molecular weight curt off for 2 hours in 1×PBS, pH7.4. The resulting dialyzed sample containing the formulated mRNA/gRNA is diluted to the desire concentration and applied directly on cells in vitro or inject tail vein or intraperitoneal for in vivo delivery. The formulated mRNA/gRNA may be stored at 4° C.

For Cas9 mRNA transfection with cell culture such as 293 cells, 0.5 μg mRNA may be added to 25 μl of Opti-MEM, followed by addition of 50-100 ng of dephosphorylated gRNA. Meanwhile, two μl of LIPOFECTAMINE™ 3000 or RNAiMax may be diluted into 25 μl of Opti-MEM and then mixed with mRNA/gRNA sample. The mixture may be incubated for 15 minutes prior to addition to the cells.

Any number of conditions may be altered to enhance the introduction of CRISPR system components into cells. Exemplary incubation conditions are pH, ionic strength, cell type, energy charge of the cells, the specific CRISPR system components present, the ratio of CRISPR system components (when more than one CRISPR system component is present), the CRISPR system component/cell ratio, concentration of cells and CRISPR system components, incubation times, etc.

One factor that may be varied, especially when CRISPR complexes are formed, is ionic strength. Ionic strength is the total ion concentration in solution. CRISPR complexes are formed from the association of CRISPR protein with CRISPR RNA and this association is partially dependent upon the ionic strength of the surrounding environment. One method for calculating the ionic strength of a solution is by the Debye and Huckel formula. In many instances, the ionic strength of solutions used in the practice of the invention will be from about 0.001 to about 3 (e.g., from about 0.001 to about 2, from about 0.001 to about 1.5, from about 0.001 to about 1, from about 0.001 to about 0.7, from about 0.001 to about 0.5, from about 0.001 to about 0.25, from about 0.001 to about 0.1, from about 0.01 to about 1, from about 0.01 to about 0.5, from about 0.01 to about 0.2, from about 0.01 to about 0.1, etc.).

pH is another factor that may affect transfection efficiency. Typically, complexation and/or transfection will occur at near physiological pH (e.g., pHs from about 6.5 to about 7.5, pHs from about 6.8 to about 7.5, pHs from about 6.9 to about 7.5, pHs from about 6.5 to about 7.3, pHs from about 6.5 to about 7.1, pHs from about 6.8 to about 7.2, etc.). In some instances, transfection efficiency is known to be sensitive to small variations in pH (e.g., =/−0.2 pH units).

The ratio of CRISPR system components to each other and to other mixture components (e.g., cells) also affects the efficiency of CRISPR system component cellular update. Using Cas9 protein and guide RNA for purposes of illustration, Cas9 protein may be complexed with guide RNA before contact with a cell or simultaneously with cellular contact. In many instances, CRISPR protein and CRISPR RNA components will be present in set ratios (e.g., 1:1, 1.5:1, 2:1, 2.5:1, 3:1, 1:1.5, 1:2, 1:2.5, 1:3, from about 0.2:1 to about 4:1, from about 0.2:1 to about 3:1, from about 0.2:1 to about 2:1, from about 0.5:1 to about 6:1, from about 0.5:1 to about 4:1, etc.). One useful ratio for Cas9 protein to guide RNA is 1:1, where each Cas9 protein has available to it one guide RNA molecular partner for complex formation.

The uptake of CRISPR complexes by cells is partially determined by the concentration of the CRISPR complexes and the cell density and the ratio of the CRISPR complexes to the cells. Typically, high CRISPR complex concentrations will result in higher amounts of uptake by available cells. Exemplary CRISPR complex/cell density conditions include 10⁷ CRISPR complexes per cell. Additionally, CRISPR complexes per cell may be in the range of 10² to 10¹² complexes per cell (e.g., from about 10² to about 10¹¹, from about 10² to about 10¹⁰, from about 10² to about 10⁹, from about 10² to about 10⁸, from about 10² to about 10⁷, from about 10² to about 10⁶, from about 10³ to about 10¹², from about 10⁴ to about 10¹², from about 10⁵ to about 10¹², from about 10⁶ to about 10¹², from about 10⁷ to about 10¹², from about 10⁸ to about 10¹², from about 10³ to about 10¹⁰, from about 10⁴ to about 10¹⁰, from about 10⁵ to about 10¹¹, etc.). Also, the cell density will typically be about 10⁵ cells per ml. Typically, cell density will be in the range of 10² to 10⁸ cells per ml (e.g., from about 10² to about 10⁷, from about 10² to about 10⁶, from about 10² to about 10⁵, from about 10² to about 10⁴, from about 10³ to about 10⁸, from about 10³ to about 10⁷, from about 10⁴ to about 10⁷, etc.).

The invention includes methods in which one or both of the CRISPR complex/cell density and/or the total cell density are adjusted such that, when double-stranded target locus cutting is assayed, the percentage of target loci cut are between 80 and 99.9% (e.g., from about 80% to about 99%, from about 85% to about 99%, from about 90% to about 99%, from about 95% to about 99%, from about 96% to about 99%, from about 80% to about 95%, from about 90% to about 97%, etc.).

One exemplary set of conditions that may be use is where ˜5⁵ cells are contacted with 500 ng of Cas9 (˜2¹² molecules) complexed with target locus specific guide RNA.

The invention also includes compositions and methods for storing reagent for intracellular genetic modification. For example, CRISPR complexes, gRNA and Cas9 protein alone, Cas9 protein and LIPOFECTAMINE® RNAiMax transfection reagent, or gRNA, Cas9 protein and OPTI-MEM® culture medium may be stored at 4° C. or frozen. In many cases, high levels of functional activity may be retained for three months with freezing. Similar results may be observed with Cas9 protein and OPTI-MEM® culture medium. The invention thus includes high-throughput reagents containing CRISPR system components. In some embodiments, such components comprise one or more of the following: one or more gRNA, one or more Cas9 protein, one or more cell culture medium (e.g., one or more mammalian cell culture medium), one or more transfection reagent, and one or more donor nucleic acid molecule.

For purposes of illustration, the invention includes multi-well plates, as well as high throughput methods employing such plates, in which different wells contain Cas9 protein and a transfection reagent. Further, different wells contain different gRNA molecules. Such plates may be used in high throughput methods for altering multiple genetic sites within cells. Each well may further contain, for example, donor DNA with termini homologous to the gRNA directed cleavage site for alteration of different loci within cells.

The invention also includes CRISPR system reagents that remain stable when stored for specified periods of time. For purposes of illustration, the invention provides CRISPR system reagents that retain at least 75% (e.g., from about 75% to about 100%, from about 80% to about 100%, from about 85% to about 100%, from about 90% to about 100%, from about 95% to about 100%, from about 75% to about 90%, from about 80% to about 90%, etc.) of their original CRISPR related activity after 3 months of storage at −20° C. Of course, CRISPR system reagents may be stored at different temperatures (e.g., 4° C., −20° C., −70° C., from about 4° C. to about −70° C., from about −20° C. to about −70° C., etc.). Further, the invention also includes CRISPR system reagents and method for storing such reagents where at least 75% of their original CRISPR related activity after up to 1 year (e.g., from about 1 month to about 12 months, from about 2 months to about 12 months, from about 3 months to about 12 months, from about 4 months to about 12 months, from about 5 months to about 12 months, from about 1 months to about 9 months, from about 3 months to about 9 months, from about 2 months to about 6 months, etc.).

In some instances, CRISPR complexes may not be stable during storage, especially under certain conditions. For example, under some conditions Cas9, gRNA and transfection reagents may be stable under one set of conditions but not under another set of conditions. It has been determined that under some conditions (e.g., in certain buffer formulations), Cas9, gRNA and transfection reagent mixtures are not stable upon freezing but are stable upon storage at 4° C. The invention this includes compositions that are stable under on set of storage conditions but not another set of storage conditions.

RNA may be prepared in water but EDTA (e.g., 0.1 mM) and/or sodium acetate buffer may be used. Cas9 protein may be prepared in 15 mM Tris HCl, 250 mM NaCl, 0.6 mM TCEP, 50% glycerol, pH 8.

Storage data may be generated using reagent mixtures contained in wells of multiwall plates. Cas9 protein may be present in wells in an amount of 500 ng/well (0.5 μl of a 0.5 μg/μl stock solution) and gRNA may be present in an amount of 200 ng/well (0.7 μl of a 300 ng/μl stock solution). All reagents may be stored as 4× solutions. Cas9/gRNA samples may be placed under storage conditions as 1.2 μl aliquots in each well. Cas9/gRNA/OPTI-MEM® samples may be placed under storage conditions as 20 μl aliquots in each well with 18.8 μl of OPTI-MEM® being present in each well. Cas9/OPTI-MEM® samples may be placed under storage conditions as 10 μl aliquots in each well with 9.5 μl of OPTI-MEM® being present in each well. Cas9/RNAiMax samples may be placed under storage conditions as 6.5 μl aliquots in each well with 6 μl of RNAiMax transfection reagent being present in each well.

The above reagents may be then used after storage in cleavage assay after being combined with additional reagents and cells. 1.2 μl of Cas9 protein and may be combined with gRNA with 18.8 μl OPTI-MEM® which may be incubated at room temperature for 5 minutes. 6 μl of LIPOFECTAMINE® RNAiMax may be also mixed with 14 μl of OPTI-MEM® which may be incubated at room temperature for 5 minutes. These two mixtures may be then combined and incubated at room temperature for 5 minutes then contacted with cells. Cas9/gRNA/OPTI-MEM® samples may be combined with 6 μl LIPOFECTAMINE® RNAiMax and 14 μl OPTI-MEM® that had been incubated at room temperature for 5 minutes. These two mixtures may be then combined and incubated at room temperature for 5 minutes then contacted with cells. Cas9/OPTI-MEM® samples may be mixed with both 0.7 μl or gRNA and 9.3 μl of OPTI-MEM® (incubated for 5 minutes at room temperature) and 6 μl LIPOFECTAMINE® RNAiMax and 14 μl OPTI-MEM® incubated for 5 minutes at room temperature), then contacted with cells after incubation for 5 minutes at room temperature. Cas9/LIPOFECTAMINE® RNAiMax samples may be mixed with 0.7 μl gRNA and 32.8 μl OPTI-MEM® (incubated at room temperature for 5 minutes), then contacted with cells.

For transfection, 293FT cells may be seeded one day prior to transfection at 20,000 cells per well in a 96 well plate format to get around 50% to 60% cell confluency on the day of transfection. Each well at the time of seeding may have 100 μl of cell culture media (DMEM, 10% FBS, and 5% each of sodium pyruvate, non-essential amino acids and GLUTAMAX™). At the time of transfection 10 μl of final transfection mix (containing Cas9, gRNA, LIPOFECTAMINE® RNAiMAX and OPTIMEM®) may be added to each well in 96 well format. Following incubation at 37° C. for 72 hours the cells may be harvested for measuring % cleavage efficiency at the respective target loci (in this case HPRT gene target) using GENEART™ cleavage detection assay.

In one aspect, the invention relates to compositions and methods related to ready to use reagents. A ready to use reagent may be in any number of forms. For example, a ready to use reagent may contain one or more Cas9 protein, one or more gRNA, one or more transfection reagent, and one or more cell culture medium. As specific example is a reagent that contains a Cas9 protein, two gRNAs, and LIPOFECTAMINE® RNAiMax all in 2× concentration and OPTI-MEM® culture medium in a 1× concentration. A ready to use reagent of this type may be mixed 1:1 with cells contained in OPTI-MEM® culture medium to yield a transfection reaction mixture for the introduction of two gRNAs into the cells, where the gRNAs share sequence homology with two locations in the genome of the cells. If appropriate, the cells may simultaneously or subsequently be contacted with one or more nucleic acid molecules for insertion into the genomic cut sites.

Another example of a ready to use reagent includes a combination of one or more Cas9 protein, one or more gRNA, and one or more cell culture medium. As specific example is a reagent that contains a Cas9 protein and two gRNAs in 2× concentration and OPTI-MEM® culture medium in a 1× concentration. A ready to use reagent of this type may be mixed first with LIPOFECTAMINE® RNAiMax and then 1:1 with cells contained in OPTI-MEM® culture medium to yield a transfection reaction mixture for the introduction of two gRNAs into the cells.

Ready to use reagents such as those set out above may be stored at 4° C. for a period of time prior to use. As noted elsewhere herein, under some conditions, Cas9, gRNA and transfection reagent mixtures are not stable upon freezing but are stable upon storage at 4° C.

Ready to use reagents may be labeled with preferred storage conditions and expiration dates that are designed to reflect a specified decrease in activity (e.g., less than 80% of activity). For example, expiration dates may range from about two weeks to about one year (e.g., from about two weeks to about ten months, from about two weeks to about eight months, from about two weeks to about six months, from about two weeks to about four months, from about one month to about one year, from about one month to about ten months, from about one month to about six months, from about one month to about four months, from about three months to about one year, from about three months to about eight months, etc.).

It has also been found that, in some instances, higher concentrations of CRISPR system components result in higher stability upon storage. Thus, in some aspects, the invention includes reagents that contain greater than 50 ng/μl (e.g., from about 50 ng/μl to about 500 ng/μl, from about 100 ng/μl to about 500 ng/μl, from about 150 ng/μl to about 500 ng/μl, from about 200 ng/μl to about 500 ng/μl, from about 250 ng/μl to about 500 ng/μl, from about 300 ng/μl to about 500 ng/μl, from about 400 ng/μl to about 500 ng/μl, etc.) of gRNA. In many instances, the molar amount of Cas9 protein, when present, to gRNA will be in the range of from about 5:1 to about 1:5 (e.g., from about 5:1 to about 1:4, from about 5:1 to about 1:3, from about 5:1 to about 1:2, from about 5:1 to about 1:1, from about 5:1 to about 1:1, from about 4:1 to about 1:5, from about 5:1 to about 1:5, from about 2:1 to about 1:5, from about 1:2 to about 1:5, from about 4:1 to about 1:4, from about 3:1 to about 1:3, from about 2:1 to about 1:2, etc.).

### Non-Chemical Stabilization:

Non-chemical stabilization refers to stabilization means that do not involve chemical modification of functional components of gene altering reagents (e.g., TAL proteins, gRNA, etc.).

A number of means of non-chemical stabilization may be used in the practice of the invention. Such means include (a) temperature, (b) pH, (c) ionic strength, (d) complexation with other compounds, (e) the presents of agents that inhibit enzymes that degrade proteins and nucleic acids (e.g., nuclease, inhibitors, protease inhibitors, etc.), and (f) drying.

In some aspects the invention relates to compositions and methods for preparing exsiccated or lyophilized compositions containing gene altering reagents. Reagents that contain only small amounts of solvent (e.g., water) are generally expected to undergo few biological reactions and thus are expected to be relatively stable even at room temperature.

While a number of methods may be used to remove water from samples (e.g., centrifugation in under vacuum or partial vacuum conditions), lyophilization may be carried out according to methods known in the art. In many instances, solvent will be removed by lyophilization. An example of a protocol for lyophilization is the following: (1) Gradient temperature decrease from +20° C. to −40° C. in 5 minutes, (2) −40° C. for 3 hours, (3) gradient temperature increase from −40° C. to −10° C. in 30 minutes, (4) −10° C. for 4 hours, (5) gradient temperature increase from −10° C. to +10° C. in 15 minutes, (6)+10° C. for 2 hours, (7) gradient temperature increase from +10° C. to +30° C. in 15 minutes, and (8)+30° C. for 4-8 hours.

In many instances, glycerol and detergents will not be present in gene altering reagents for certain dry down methods. For example, while glycerol can be present in the lyophilization method referred to above, it is not preferred.

Exsiccation or drying may be employed for stabilizing gene altering reagents. Typically, greater than 80% of gene altering reagents is water. Removal of substantial portions of this water can result in stabilization. Lyophilization, for example, typically lowers the moisture content of a solution to a percentage between 0.3% and 8%. Thus, the invention includes gene altering reagents where the moisture content is from about 0.1% to about 10%, from about 0.5% to about 10%, from about 1% to about 10%, from about 0.1% to about 7%, from about 0.1% to about 5%, from about 0.5% to about 5%, from about 0.5% to about 4%, from about 0.3% to about 6%, from about 0.3% to about 4%, from about 0.3% to about 3%, etc.

One advantage of drying gene altering reagents is that this increases stability at ambient temperature. Thus, in one aspect, the invention provides methods for stabilizing gene altering reagents, as well as compositions generated by such methods.

In some embodiments, cellobiose may be present as a stabilizer at concentrations between 50 mM and 500 mM in a preparation prior to solvent removal. One of the advantage of the use of cellobiose is that it is an effective stabilizer for both lyophilization and preservation of biological molecules (e.g., nucleic acids and proteins), whereas stabilizers of the known art are generally used for either one or the other purpose. Also in many instances, a salt such as KCl or MgCl₂ will be present prior to solvent removal.

A number of means may also be employed for inhibiting the degradation of proteins. One is the presence of one or more protease inhibitors (e.g., phenylmethylsulfonyl fluoride, leupeptin, etc.).

A number of means may also be employed for inhibiting the degradation of nucleic acid molecules, including RNA molecules. One is the presence of one or more RNase inhibitors. A number of commercially available RNase inhibitors are available, including SUPERASE IN™ RNase Inhibitor (cat. no. AM2694), RNASEOUT™ (cat. no. 10777-019), and ANTI-RNase (cat. no. AM2690), all of which are available from Thermo Fisher Scientific.

Capsid proteins from viruses may also be used to stabilize nucleic acid molecules. These viruses may be DNA viruses or RNA viruses. By way of example, when one seeks to stabilize gRNA molecules, one may use capsid proteins from single-stranded RNA viruses such as Coronavirus, SARS virus, Poliovirus, Rhinovirus, and/or Hepatitis A virus.

Further, gRNA may be stabilized by complexation with Cas9 protein. Thus, the invention includes stabilized gene altering reagents containing nucleic acid/protein complexes. Further, such complexes may have solvent removed from them.

### Chemical/Base Stabilization:

Chemical stabilization refers to stabilization means that involve chemical modification of functional components of gene altering reagents (e.g., TAL proteins, gRNA, etc.).

Nucleic acid molecules used in the practice of the invention may be chemically modified. Chemical modification may be employed for a number of purposes. For example, chemical modification may be used to stabilize nucleic acid molecules (e.g., RNA molecules) during storage and/or increase their intracellular half-life. Further, with respect to functional RNA molecules (e.g., gRNA molecules) hairpins may be altered in a manner that stabilizes their structure. This can be done by selection of bases that enhance the formation of hairpin (e.g., G/C content).

Chemical modifications may be of any number of chemical groups and locations. The suitability of a particular chemical modification will vary with the type of RNA molecule and the location within the RNA molecule of the chemical group.

Chemical modifications may be of bases or inter base linkages. Exemplary chemical modifications may include phosphorothioate modifications, 2′-O-methyl modifications, 2′-O-propyl modifications, 2′-O-ethyl modifications, 2′-fluoro modifications, and/or a combination of such modifications. Modified sugars may also be used. Modified sugars include D-ribose, dideoxynucleotides, 2′-O-alkyl (including 2′-O-methyl and 2′-O-ethyl), i.e., 2′-alkoxy, 2′-amino, 2′-S -alkyl, 2′-halo (including 2′-fluoro), 2′-methoxyethoxy, 2′-allyloxy (—OCH2CH═CH2), 2′-propargyl, 2′-propyl, ethynyl, ethenyl, propenyl, and cyano and the like.

Additional chemical modifications that may be used in the practice of the invention may be found in Hendel et al., Nature Biotech. doi:10.1038/nbt.3290 (2015) and Radhar et al., Proc. Nat'l. Acad. Sci. (USA) doi/10.1073/pnas.1520883112 (2015).

Chemical modifications also include phosphodiester analogs, such as, phosphorothioate, phosphorodithioate, and P ethyoxyphosphodiester, P-ethoxyphosphodiester, P-alkyloxyphosphotriester, methylphosphonate, and nonphosphorus containing linkages (e.g., acetals and amides).

Pseudouridine is the C-glycoside isomer of the uridine and, of the over one hundred different modified nucleosides found in RNA, it is the most prevalent. Pseudouridine is formed by enzymes called pseudouridine synthases, which post-transcriptionally isomerize specific uridine residues in RNA in a process termed pseudouridylation. Pseudouridine is suggested provide protection from radiation. RNA molecules may be stabilized by the addition of pseudouridine and/or 2′-O-methyl modifications at one or more location at or near the 5′ and/or 3′ termini.

Chemical modifications may be increase the storage life and/or intracellular half-life by anywhere from 1.2 to 20 fold (e.g., from about 1.5 to about 20, from about 2 to about 20, from about 1.5 to about 20, from about 1.5 to about 20, from about 1.5 to about 20, from about 1.5 to about 20, from about 1.5 to about 20, from about 1.5 to about 20, etc.).

Chemical modifications may be located at one terminus, both termini and/or interior in nucleic acid molecules. In many instances, chemical modifications will be positioned to inhibit digestion of nucleic acid molecules by exonucleases. In some formats, from one to ten (e.g., from about one to about nine, from about one to about six, from about one to about five, from about one to about four, from about one to about three, from about one to about two, etc.) terminal 5′ and/or 3′ bases will be chemically modified. In more specific formats, the chemical modifications will be either phosphorothioate modifications or 2′-O-methyl modifications or a combination of these modifications.

Chemical modifications may be present in a number from one to twenty (e.g., from about one to about fifteen, from about two to about fifteen, from about three to about fifteen, from about three to about ten, from about three to about eight, from about two to about five, etc.) modifications, such as base modifications, linker modifications and/or sugar modifications.

Many exonucleases are processive in the sense that they remaining attached to their substrates and performing multiple rounds of catalysis before dissociating. Termini of RNA molecules may have different groups present to prevent degradation. As examples, synthetic RNA typically has a 5′ hydroxyl group. RNA produced by in vitro transcription typically has a 5′ triphosphate group. Natural RNA typically has a 5′monophosphate group. The invention includes stabilized RNA molecules that have one or more of the se 5′ groups, as well as other 5′ groups. As an example, 5′ triphosphate groups may be converted to monophosphate groups by using RNA 5′ pyrophosphohydrolase. Further, 5′ monophosphate groups may be used to improve RNA stability.

A number of additional means may be used to stabilize nucleic acid molecules. For example, a string of polyGs may be added to the 3′ terminus of a nucleic acid molecule to inhibit degradation. In particular, a polyG region may be present in the place of polyA regions found at the 3′ end of mRNA, resulting in increased intracellular half-life on the RNA molecules.

Another way to improve stability of RNA molecules (e.g., gRNA molecules) is to provide these molecules as stabilized loops or hairpins. One example of a modification of such loops is those with contain C/G rich regions. The three hydrogen bonds between these bases in creases loop stability, as compared to loops formed from nucleic acid segments having A/T bases. Stability of RNA molecules can also be increased by the addition of loops, such as tetraloops composed of four pairs of C/G bases. Loops may also be stabilized or introduced as one or both termini. In the case of gRNA, a loop may be introduced at the 5′ terminus. The invention thus includes nucleic acid molecules that contain hairpin regions wherein between 60% to 100% (e.g., from about 65% to about 100%, from about 70% to about 100%, from about 75% to about 100%, from about 80% to about 100%, from about 75% to about 90%, etc.) of the paired bases are C/G pairs. Further, these hairpin regions may contain from about 4 to about 20 paired bases (e.g., from about 5 to about 20 paired bases, from about 6 to about 20 paired bases, from about 7 to about 20 paired bases, from about 5 to about 15 paired bases, from about 6 to about 14 paired bases, etc.).

In some instances, the number of naturally resident hairpins present may be changed to enhance stability of a nucleic acid molecule. The natural tracr molecule forms three hairpins. The final hairpin has 3-5 bases additional at the 3′ end. Tracr molecules, as well as other RNA molecules (e.g., gRNA molecules), may be stabilized by removing some or all of these terminal bases. This is believed to inhibit nuclease initiation. Further, truncation of naturally resident hairpins may result in stabilized RNA molecules by changing solvent exposure.

RNA molecules may also be formed through the introduction of regions that form triplex and/or quadraplex structures, especially at or near the 3′ terminus.

Cross-link groups (e.g., photo-activatable groups) can be added to gRNA (e.g., at or near the 3′ terminus) that allow for cross-linking to the Cas9 protein. This allows for the formation of a stable gRNA/Cas9 complex, where the gRNA is believed to be protected from degradation by the protein.

### Gene Alteration Activities:

Reagents of the invention can have any number of activities. For example, the reagents may comprise fusion proteins that have one or more heterologous domains (e.g., one, two, three, four, five, etc.). Fusion proteins may comprise any additional protein sequence, and optionally a linker sequence between any two domains. Examples of protein domains that may be a fusion protein component include, without limitation, epitope tags, reporter gene sequences, and one or more domain having one or more of the following activities: methylase activity, demethylase activity, transcription activation activity, transcription repression activity, transcription release factor activity, histone modification activity (e.g., acetylation activity, deacetylation activity, phosphorylation activity, dephosphorylation activity, methylation activity, demethylation activity, etc.) RNA cleavage activity, and nucleic acid binding activity.

In particular, provided herein, in part, are gene altering reagents, which comprise at least one nuclear localization signal, at least one domain with a functional activity (e.g., nuclease, methylase, etc.), and at least one domain that interacts with a target locus or at least one domain that interaction with a nucleic acid molecule that interacts with a target locus.

Gene altering reagents may be employed to activate or repress transcription. For example, “dead” Cas9 (i.e., dCas9) proteins without nuclease activity may be used for non-code altering purposes. dCas9-transcriptional activator fusion protein (e.g., dCas9-VP64) may be used in conjunction with a guide RNA to activate transcription of nucleic acid associated with a target locus. Similarly, dCas9-repressor fusions (e.g., dCas9-KRAB transcriptional repressor) may be used to repress transcription of nucleic acid associated with a target locus. Transcriptional activation and repression such as the referred to above are discussed in, for example, Kearns et al., Cas9 effector-mediated regulation of transcription and differentiation in human pluripotent stem cells, Development, 141:219-223 (2014).

The invention thus includes compositions and methods for the production and use of gene altering reagents for the activation and repression of transcription.

FIG. 7 shows the selection of two closely associated sites that form a target locus. Each of the sites (Site 1 and Site 2) binds a gene altering reagent with nicking activity. One purpose of this is to minimize “off target” cutting of nucleic acid.

The two sites exemplified in FIG. 7 will generally be located sufficiently close to each other so that the double-stranded nucleic acid containing the nick breaks. While this distance will vary with factors such as the AT/CG content of the region, the nick sites will generally be within 200 base pairs of each other (e.g., from about 1 to about 200, from about 10 to about 200, from about 25 to about 200, from about 40 to about 200, from about 50 to about 200, from about 60 to about 200, from about 1 to about 100, from about 10 to about 100, from about 20 to about 100, from about 30 to about 100, from about 40 to about 100, from about 50 to about 100, from about 1 to about 60, from about 10 to about 60, from about 20 to about 60, from about 30 to about 60, from about 40 to about 60, from about 1 to about 35, from about 5 to about 35, from about 10 to about 35, from about 20 to about 35, from about 25 to about 35, from about 1 to about 25, from about 10 to about 25, from about 15 to about 25, from about 2 to about 15, from about 5 to about 15, etc. base pairs).

The nicking activity may be accomplished in a number of ways. For example, when the gene altering reagent is Cas9, the Cas9 protein has two domains, termed RuvC and HNH, that nick different strands of double-stranded nucleic acid. Cas9 proteins may be altered to inactivate one domain or the other. The result is that two Cas9 proteins are required to nick the target locus in order for a double-stranded break to occur. For example, an aspartate-to-alanine substitution (D10A) in the RuvC catalytic domain of Cas9 from S. pyogenes converts Cas9 from a nuclease that cleaves both strands to a nickase (cleaves a single strand). Other examples of mutations that render Cas9 a nickase include H840A, N854A, and N863A.

CRISPR proteins (e.g., Cas9) with nickase activities may be used in combination with guide sequences (e.g., two guide sequences) which target respectively sense and antisense strands of the DNA target.

Another way to generate double-stranded breaks in nucleic acid using nickase activity is by using CRISPR proteins that lack nuclease activity linked to a heterologous nuclease domain. One example of this is a mutated form of Cas9, referred to as dCas9, linked to FokI domain. FokI domains require dimerization for nuclease activity. Thus, in such instances, CRISPR RNA molecules are used to bring two dCas9-FokI fusion proteins into sufficiently close proximity to generate nuclease activity that results in the formation of a double-stranded cut. Methods of this type are set out in Tsai et al., “Dimeric CRISPR RNA-guided FokI nucleases for highly specific genome editing,” Nature Biotech., 32:569-576 (2014) and Guilinger et al., “Fusion of catalytically inactive Cas9 to FokI nuclease improves the specificity of genome modification,” Nature Biotech., 32:577-582 (2014).

Another way to minimize “off target” cutting of nucleic acid is through the use of nucleases that are inactive until they dimerize. One example of such a nuclease is FokI. Zinc finger proteins and TAL effector proteins have been designed to bind different sites on a nucleic acid molecule to allow for the FokI domains to dimerize, resulting in reconstitution of nuclease activity.

The invention thus includes gene altering reagents that recognize more than one locus on a nucleic acid molecule. In many instances, the distance between the recognition sites will be in the same range as the nick sites referred to in reference to FIG. 7.

Functional activities can be measured in any number of ways. For example, activities based upon induction or repression of expression can be measured by assessing increases or decreases in transcription and/or translation.

When functional activities related to the cleavage of DNA (e.g., intracellular DNA), then a number of commercial products are available for the detection of nucleic acid cleavage. One such product is the GENEART® Genomic Cleavage Detection Kit (cat. no. A24372), available from Thermo Fisher Scientific. Additional assay may be found in U.S. patent application Ser. No. 14/879,872, filed Oct. 9, 2015, entitled “CRISPR Oligonucleotides and Gene Editing, the entire disclosure of which is incorporated herein by reference.

### Reagent Mixtures and Formats:

A number of compounds that do not have direct gene alteration activity may be included in the reagent mixture. One such set of compounds is transfection reagents. These may be included to for minimal addition to the gene altering reagent as part of an experimental protocol.

Some transfection agents suitable for use with the invention include transfection agents that facilitate the introduction of RNA, DNA and proteins into cells and are set out elsewhere herein.

Gene altering reagents may be set up in a format such that minimal additions are required for gene altering activity. In one exemplary format, donor nucleic acid, a pair of ZNF-FokI fusion proteins, and a transfection reagent are lyophilized in a well of a 96 well plate. Cells in a culture medium are added to the well with the lyophilized gene altering reagent and another well that does not contain the gene altering reagent (a control well). The efficiency of homologous recombination at the target locus is later measured for both samples.

In some instances, the gene altering reagent will contain gRNA and Cas9 protein will be expressed by cells contacted with the gRNA. gRNA taken up by the cells will then associate with Cas9 protein expressed intracellularly to reconstitute gene altering activities. Where appropriate, these cells may be contacted with donor nucleic acid prior to, simultaneously with, or after the cells have been contacted with gRNA.

The invention further includes collections of gene altering reagents with specificity for individual target sites. For example, the invention includes collections of gene altering reagents with specificity for target sites within particular types of cell (e.g., human cells). The members of such collection of cells may be generated based upon sequence information for these particular types of cells. As an example, one such collection could be generated using the complete genome sequence of a particular type of cell. The genome sequence data can be used to generate a library of gene altering reagents with specificity for the coding region of each gene within the human genome.

Collections or libraries of crRNA molecules or the invention may include a wide variety of individual molecules such as from about five to about 100,000 (e.g., from about 50 to about 100,000, from about 200 to about 100,000, from about 500 to about 100,000, from about 800 to about 100,000, from about 1,000 to about 100,000, from about 2,000 to about 100,000, from about 4,000 to about 100,000, from about 5,000 to about 100,000, from about 50 to about 50,000, from about 100 to about 50,000, from about 500 to about 50,000, from about 1,000 to about 50,000, from about 2,000 to about 50,000, from about 4,000 to about 50,000, from about 50 to about 10,000, from about 100 to about 10,000, from about 200 to about 10,000, from about 500 to about 10,000, from about 1,000 to about 10,000, from about 2,000 to about 10,000, from about 4,000 to about 10,000, from about 50 to about 5,000, from about 100 to about 5,000, from about 500 to about 5,000, from about 1,000 to about 5,000, from about 50 to about 2,000, from about 100 to about 2,000, from about 500 to about 2,000, etc.).

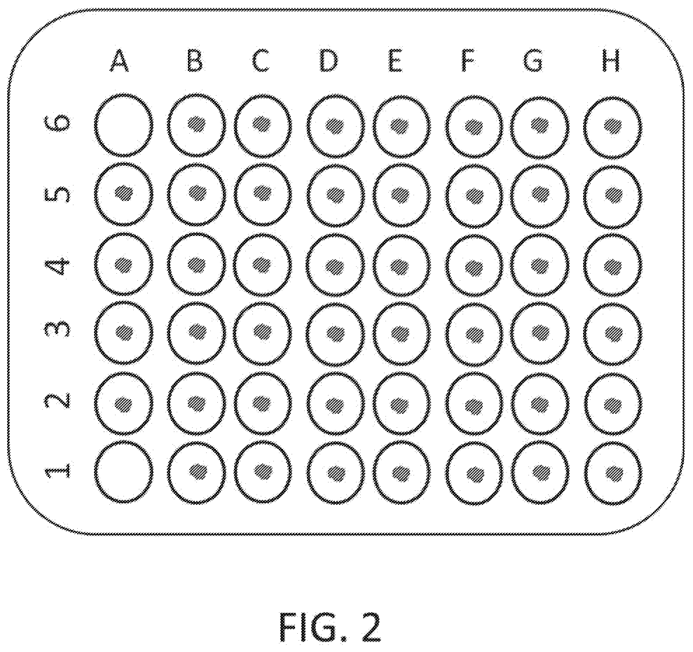

Gene altering reagents used in the practice of the invention may be stored in a number of different formats. For example, RNA molecules may be stored in tubes (e.g., 1.5 ml microcentrifuge tubes) or in the wells of plates (e.g., 96 well, 384 well, or 1536 well plates). One exemplary format is shown in FIG. 2. In this figure, wells A,1 and A,6 are control wells and contain no gene altering reagents. The other wells contain desiccated gene altering reagents that may be reconstituted with, for example, culture media containing cells. Further, each well may contain a gene altering reagent with binding specificity for a different target locus.

### Vector Components and Cells:

A number of functional nucleic acid components (e.g., promoters, polyA signal, origins of replication, selectable markers, etc.) may be used in the practice of the invention. The choice of functional nucleic acid components used in the practice of the invention, when employed, will vary greatly with the nature of the use and the specifics of the system (e.g., intracellular, extracellular, in vitro transcription, coupled in vitro transcription/translation, etc.).

Promoter choice depends upon a number of factors such as the expression products and the type of cell or system that is used. For example, non-mRNA molecules are often produced using RNA polymerase I or III promoters. mRNA is generally transcribed using RNA polymerase II promoters. There are exceptions, however. One is microRNA expression systems where a microRNA can be transcribed from DNA using an RNA polymerase II promoter (e.g., the CMV promoter). While RNA polymerase II promoters do not have “sharp” stop and start points, microRNAs tend to be processed by removal of 5′ and 3′ termini. Thus, “extra” RNA segments at the termini are removed. mRNA (e.g., cas9 mRNA) is normally produced via RNA polymerase II promoters.

The choice of a specific promoter varies with the particular application. For example, the T7, T3 and SP6 promoters are often used for in vitro transcription and in vitro transcription/translations systems. When intracellular expression in desired, the promoter or promoters used will generally be designed to function efficiently within the cells employed. The CMV promoter, for example, is a strong promoter for use within mammalian cells. The hybrid Hsp70A-Rbc S2 promoter is a constitutive promoter that functions well in eukaryotic algae such as Chlamydomonas reinhardtii. (see the product manual “GeneArt® Chlamydomonas Protein Expression Kit”, cat. no. A24244, version B.0, from Life Technologies Corp., Carlsbad, Calif.). Additional promoters that may be used in the practice of the invention include AOX1, GAP, cauliflower mosaic virus 35S, pGC1, EF1α, and Hsp70 promoters.

The DNA segment in the expression vector is operatively linked to an appropriate expression control sequence(s) (promoter) to direct RNA synthesis. Suitable eukaryotic promoters include the CMV immediate early promoter, the HSV thymidine kinase promoter, the early and late SV40 promoters, the promoters of retroviral LTRs, such as those of the Rous Sarcoma Virus (RSV), and metallothionein promoters, such as the mouse metallothionein-I promoter. Exemplary promoters suitable for use with the invention are from the type III class of RNA polymerase III promoters. Additionally, the promoters may be selected from the group consisting of the U6 and H1 promoters. The U6 and H1 promoters are both members of the type III class of RNA polymerase III promoters.

Cells suitable for use with the present invention include a wide variety of prokaryotic and eukaryotic cells. In many instances, one or more CRISPR system components will not be naturally associated with the cell (i.e., will be exogenous to the cell).

Representative cells that may be used in the practice of the invention include, but are not limited to, bacterial cells, yeast cells, plant cells and animal cells. Exemplary bacterial cells include Escherichia spp. cells (particularly E. coli cells and most particularly E. coli strains DH10B, Stb12, DH5α, DB3, DB3.1), Bacillus spp. cells (particularly B. subtilis and B. megaterium cells), Streptomyces spp. cells, Erwinia spp. cells, Klebsiella spp. cells, Serratia spp. cells (particularly S. marcessans cells), Pseudomonas spp. cells (particularly P. aeruginosa cells), and Salmonella spp. cells (particularly S. typhimurium and S. typhi cells). Exemplary animal cells include insect cells (most particularly Drosophila melanogaster cells, Spodoptera frugiperda Sf9 and Sf21 cells and Trichoplusa High-Five cells), nematode cells (particularly C. elegans cells), avian cells, amphibian cells (particularly Xenopus laevis cells), reptilian cells, and mammalian cells (more particularly NIH3T3, CHO, COS, VERO, BHK CHO-K1, BHK-21, HeLa, COS-7, HEK 293, HEK 293T, HT1080, PC12, MDCK, C2C12, Jurkat, NIH3T3, K-562, TF-1, P19 and human embryonic stem cells like clone H9 (Wicell, Madison, Wis., USA)). Exemplary yeast cells include Saccharomyces cerevisiae cells and Pichia pastoris cells. These and other cells are available commercially, for example, from Thermo-Fisher Scientific (Waltham, Mass.), the American Type Culture Collection, and Agricultural Research Culture Collection (NRRL; Peoria, Ill.). Exemplary plant cells include cells such as those derived from barley, wheat, rice, soybean, potato, arabidopsis and tobacco (e.g., Nicotiana tabacum SR 1).

### Kits:

The invention also provides kits for, in part, the assembly and/or storage of nucleic acid molecules and for the editing of cellular genomes. As part of these kits, materials and instruction are provided for both the assembly of nucleic acid molecules and the preparation of reaction mixtures for storage and use of kit components.

Kits of the invention will often contain one or more of the following components:

1. One or more nucleic acid molecule (e.g., one or more primer, one or more DNA molecule encoding Cas9, dCas9, guide RNA, etc., one or more mRNA encoding a CRISPR system component, such as Cas9, dCas9, etc.),

2. One or more polymerase,

3. One or more protein (e.g., one or more CRISPR protein such as Cas9, dCas9, etc.),

4. One or more phosphatase,

5. One or more partial vector (e.g., one or more nucleic acid segment containing an origin of replication and/or a selectable marker) or complete vector, and

6. Instructions for how to use kits components.

In particular, some kits of the invention may include one or more of the following: (a) a double-stranded nucleic acid molecule encoding the 3′end of a guide RNA molecule (see FIG. 12), wherein this double-stranded nucleic acid molecule does not encode all or part of Hybridization Region 1, (b) a polymerase, and (c) at least one buffer.

In some embodiments, kits may comprise one or more reagents for use in a process utilizing one or more of the CRISPR system components discussed herein or for producing one or more CRISPR system component discussed herein.

Kit reagents may be provided in any suitable container. A kit may provide, for example, one or more reaction or storage buffers. Reagents may be provided in a form that is usable in a particular reaction, or in a form that requires addition of one or more other components before use (e.g., in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10.

Some of the examples below, as well as other material set out herein, may also be found in U.S. application Ser. No. 14/879,872, filed Oct. 9, 2015, titled “CRISPR Oligonucleotides and Gene Editing” and 62/257,951, filed Nov. 20, 2015, titled “Stabilized Reagents for Genome Modification”. The disclosures of these two U.S. patent applications are incorporated herein by reference in their entirety.

### EXAMPLES

### Example 1: One Step Synthesis of gRNA Template and High Efficiency Cell Engineering Workflow

Abstract

CRISPR-Cas9 systems provide innovative applications in genome engineering. To edit the genome, expression of Cas9, mature crRNA and tracrRNA or a single guide RNA (gRNA) is required. Elements of the mature crRNA and tracrRNA or a gRNA are often built into a Cas9 expression plasmid or constructed in a standard plasmid driven by a U6 promoter for mammalian expression. The rapid synthesis of gRNA template is described in this example, which combines gene synthesis and DNA fragment assembly technologies with an accuracy of assembly of >96%. In other words, over 96% of the assembled nucleic acid molecules are the desired assembly products. The method allows rapid synthesis of guide RNA (gRNA) via in vitro transcription using short DNA oligonucleotides. In conjunction with Cas9 protein delivery, Cas9/gRNA complexes can be transfected into the cells through processes such as lipid-mediated methods, electroporation, and cell penetrating peptide mediated delivery. Overall, cell engineering workflows can be reduced to at least four days and, in some instances, two days. Methods described herein are applicable for high throughput gRNA synthesis and genome-wide editing.

Introduction

CRISPR-Cas9 mediated genome engineering enables researchers to modify genomic DNA in vivo directly and efficiently. Three components (Cas9, mature crRNA and tracrRNA) are essential for efficient cell engineering. Although the mature crRNA and tracrRNA can be synthesized chemically, the quality of the synthetic RNA is often not sufficient for in vivo cell engineering due, for example, to the presence of truncated by-products. Thus, mature crRNA and tracrRNA or a combined single gRNA are often transcribed from a Cas9 expression plasmid or built into a separate plasmid driven by a U6 promoter. The resulting plasmids are then transfected or co-transfected into the cells. Because the constructs are relatively large, the delivery of plasmid DNA often becomes the limiting step, especially for suspension cells. Recently Cas9 mRNA has employed to increase the rate of DNA cleavage. To make gRNA, a pre-cloned all-in-one plasmid based upon, for example, may serve as template to prepare a gRNA PCR fragment containing a T7 promoter, followed by gel extraction. Alternatively a synthetic DNA string may be used as a template.

Overall, it is time-consuming to prepare the gRNA template for in vitro transcription. A gRNA template can be assembled via PCR in about one hour. Further, gRNA can be generated in vitro transcription in about 3 hours. DNA oligonucleotides can be converted to into gRNA in about 4 hours. A workflow with the above timing elements was tested and. Furthermore, in combination with Cas9 protein transfection technology, cell engineering cycle was accomplished as described herein in four days.

Materials and Methods

Materials

293FT cells, DMEM medium, Fetal Bovine Serum (FBS), OPTI-MEM® Medium, LIPOFECTAMINE® 3000, RNAIMAX™, MESSENGERMAX™, GENEART® CRISPR Nuclease Vector with OFP Reporter, 2% E-GEL® EX Agarose Gels, PURELINK® PCR Micro Kit, TranscriptAid T7 High Yield Transcription Kit, MEGASHORTSCRIPT™ T7 Transcription Kit, MEGACLEAR™ Transcription Clean-Up Kit, ZERO BLUNT® TOPO® PCR Cloning Kit, PURELINK® Pro Quick96 Plasmid Purification Kit, Qubit® RNA BR Assay Kit, QUBIT® Protein Assay Kit, Pierce LAL Chromogenic Endotoxin Quantitation Kit, GENEART® Genomic Cleavage Detection Kit, and POROS® Heparin column were from Thermo Fisher Scientific. PHUSION® High-Fidelity DNA Polymerase was purchased from New England Biolabs. HIPREP™ 16/60 Sephacryl S-300 HR gel filtration column was purchased from GE Healthcare. All the DNA oligonucleotides used for gRNA synthesis were from Thermo Fisher Scientific.

Methods

One Step Synthesis of gRNA Template

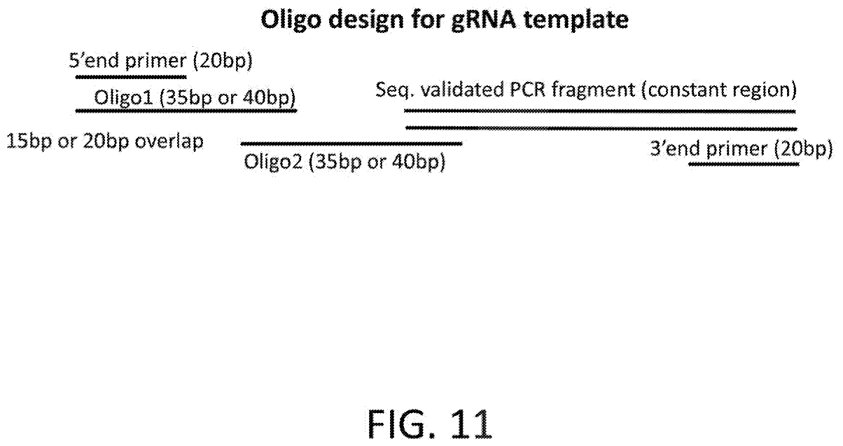

The design of oligonucleotides for the synthesis of gRNA template is depicted in FIG. 11. The forward primer:

5′-GTT TTA GAG CTA GAA ATA GCA AG-3′ (SEQ ID NO: 6) and reverse primer:

5′-AAA AGC ACC GAC TCG GTG CCA C-3′ (SEQ ID NO: 7) were used to amplify the 80 bp constant region of tracrRNA from a GENEART® CRISPR Nuclease Vector, followed by purification using agarose gel extraction. The concentration of PCR product was measured by Nanodrop (Thermo Fisher Scientific) and the molarity was calculated based on the molecular weight of 24.8 kd. To prepare a pool of oligonucleotides, an aliquot of the 80 bp PCR product was mixed with two end primers 5′-taatacgactcactatagg-3′ (SEQ ID NO: 8) and 5′-AAA AGC ACC GAC TCG GTG CCA C-3′ (SEQ ID NO: 7) with a final concentration of 0.3 μM for the 80 bp PCR product and 10 μM for each of the end primers. For a specific target, a 34 bp forward primer consisting of the 19 bp T7 promoter sequence taatacgactcactatagg (SEQ ID NO: 8) and 15 bp of the 5′end target sequence, and a 34 bp reverse primer consisting of 20 bp target sequence and 14 bp of the 5′ end tracrRNA sequence gttttagagctaga (SEQ ID NO: 9) were chemically synthesized with 15 bp overlap. A working solution containing the two 34 bp oligonucleotides was prepared at a final concentration of 0.3 μM. Alternatively, a pair of 39 bp forward and reverse primers with 20 bp overlap was synthesized and tested. To set up the one step synthesis of gRNA template, 1.5 μl of pool oligonucleotides and 1.5 μl of the working solution were added to a PCR tube containing 10 μl of 5× Phusion HF buffer, 1 μl of 10 mM dNTP, 35.5 μl H₂O, and 0.5 μl PHUSION® High-Fidelity DNA polymerase. The PCR program was set at 98° C. for 30 sec and then 30 cycles of 98° C. for 5 sec and 55° C. for sec, followed by incubation at 72° C. for 30 sec and 4° C. forever. The PCR product was analyzed by a 2% E-GEL® EX Agarose Gel, followed by purification using Purelink PCR micro column. The DNA concentration was determined by Nanodrop instrument.

To determine the error rate, the PCR product was cloned into ZERO BLUNT® TOPO® vector, followed by plasmid DNA isolation and sequencing.

In Vitro Transcription

The in vitro transcription of gRNA template was carried out using TRANSCRIPTAID™ T7 High Yield Transcription Kit. Briefly, 6 μl of gRNA template (250-500 ng) was added to a reaction mixture containing 8 μl of NTP, 4 μl of 5× reaction buffer and 2 μl of T7 enzyme mix. The reaction was carried out at 37° C. for 2 hrs, followed by incubation with DNase I (2 units per 120 ng DNA template) for 15 minutes. The gRNA product was purified using MEGACLEAR™ Transcription Clean-Up kit as described in the manual. The concentration of RNA was determined using QUBIT® RNA BR Assay Kit.

Expression and Purification of Cas9 Protein

A glycerol stock BL21(DE3) star E. coli strain expressing NLS Cas9 protein was inoculated in 20 ml BRM medium and grown overnight at 37° C. in a shaking incubator. The overnight culture was then added to 1 liter of BRM medium and grown cells to an OD₆₀₀ nm of 0.6-0.8 at 37° C. in a shaking incubator (˜4-5 hours). An aliquot of un-induced sample was taken for monitoring protein induction with IPTG. 0.5 ml of 1 M IPTG was added to the culture and incubated overnight at room temperature in a shaking incubator. An aliquot of induced sample along with un-induced sample were analyzed by SDS-PAGE. Upon validation of protein induction, the culture medium was centrifuged at 5000 rpm for 15 minutes to harvest the cell pellets (˜24 grams of wet weight). 100 ml of buffer A containing 20 mM Tris (pH7.5), 100 mM NaCl, 10% Glycerol, and 1 mM PMSF was used to resuspend the cell pellet. The cell suspension was sonicated on ice for 30 minutes with power level of medium tip set at 8, 10 sec “on”, and 20 sec “off”. The cell lysate was clarified by centrifugation at 16500 rpm for 30 minutes. The supernatant was filtered through a 0.2 μm filter device prior to loading to a 16 ml heparin column previously equilibrated with buffer A at a flow rate of 2 ml/min. The column was first washed with five column volume of buffer A and then gradually increased to 40% of buffer B containing 20 mM Tris (pH7.5), 1.2 M NaCl and 10% glycerol. The Cas9 protein was eluted with a 5 CV gradient from 40% to 100% buffer B. The fractions were analyzed by SDS-PAGE. Fractions containing Cas9 protein were combined and concentrated using two 15 ml Amicon Centrifugal filter units (EMD Millipore, Cat. No. UFC905024). The concentrated protein was filtered through a 0.2 μm filter device and loaded twice onto a 120 ml of HIPREP™ 16/60 Sephacryl S-300 HR column previously equilibrated with buffer C containing 20 mM Tris (pH 8), 250 mM KCl and 10% Glycerol. The fractions containing Cas9 protein were pooled and concentrated. The protein concentration was determined by QUBIT® Protein Assay Kit. The endotoxin level in the purified protein was measured by Endotoxin Quantitation Kit. The concentrated protein was adjusted to 50% glycerol and stored at −20° C.

Cell Culture

293FT cells were maintained in DMEM medium supplemented with 10% FBS in a 5% CO₂ incubator. One day prior to transfection, the cells were seeded in a 24-well plate at a cell density of 2.5×10⁵ cells/0.5 ml medium. For transfection, 500 ng of purified Cas9 was added to 25 μl of OPTI-MEM® medium, followed by addition of 120 ng gRNA. The sample was mixed by gently tapping the tubes a few times and then incubated at room temperature for 10 minutes. To a separate test tube, 3 μl of RNAIMAX™ was added to 25 μl of OPTI-MEM® medium. The diluted transfection reagent was transferred to the tube containing Cas9 protein/gRNA complexes, followed by incubation at room temperature for 15 minutes. The entire solution was then added to the cells in a 24-well and mixed by gently swirling the plate a few times. The plate was incubated at 37° C. for 48 hours in a 5% CO₂ incubator. The percentage of genome editing was measured by GENEART® Genomic Cleavage Detection Kit.

Results and Discussion

One Step Synthesis of gRNA Template

Since gRNA synthesis is one of the limiting steps in genome engineering, an attempt was made to reduce the time for gRNA synthesis. As an example, HPRT-T1 target catttctcagtcctaaaca (SEQ ID NO: 10) was chosen, but these methods were also found to work for GFP and VEGFA-T3 targets (data not shown). Initially, a gene synthesis approach was utilized to assemble a gRNA template using a set of 6 synthetic DNA oligonucleotides (Set 1 oligonucleotides in Table 7). Through optimization of oligonucleotide pool concentration and PCR condition, a clean PCR product was obtained on an agarose gel (data not shown). An aliquot of the PCR product served as template to synthesize the gRNA via in vitro transcription. The quality of synthetic gRNA was analyzed by a denaturing gel.

To test the functionality of synthetic gRNA, gRNA was associated with Cas9 protein. The resulting complexes were then delivered to the 293FT cells via lipid-based transfection. However, the evaluation of in vivo genome cleavage assay indicated that gRNA did not work well (data not shown). To determine the problem, gRNA templates were cloned into a ZERO BLUNT® TOPO vector and then sequenced. It was observed that more than 20% of the gRNA templates harbored mutations, mostly deletions. To minimize the potential sources of errors, instead of using long synthetic oligos to create the complete T7 promoter/guide RNA template, the constant 80 bp tracrRNA region was amplified from a sequence-validated plasmid template, followed by gel purification to remove the template. Then to fuse the T7 promoter sequence and target sequence to the constant tracrRNA, a pair of 34 bp or 39 bp forward and reverse oligonucleotides that share 15 bp or 20 bp homology, respectively, across the variable target sequence were designed, wherein the middle oligo 2 also shared 19 bases of homology with the tracrRNA region.

As described in Materials and Methods, the gRNA template was assembled in a single PCR reaction using a pool of DNA oligonucleotides and tracrRNA fragment (FIG. 11). Upon PCR micro column purification, the gRNA template was used to prepare gRNA via in vitro transcription. The quality of gRNA was examined using a TBE-urea denaturing gel. gRNA prepared via PCR amplification from an all-in-one plasmid was used as positive control. An “all-in-one plasmid” is a plasmid that contains all of the components of a CRISPR system, such as guide RNA and Cas9 coding sequences (e.g., Thermo Fisher Scientific, cat. nos. A21174 and A21175).

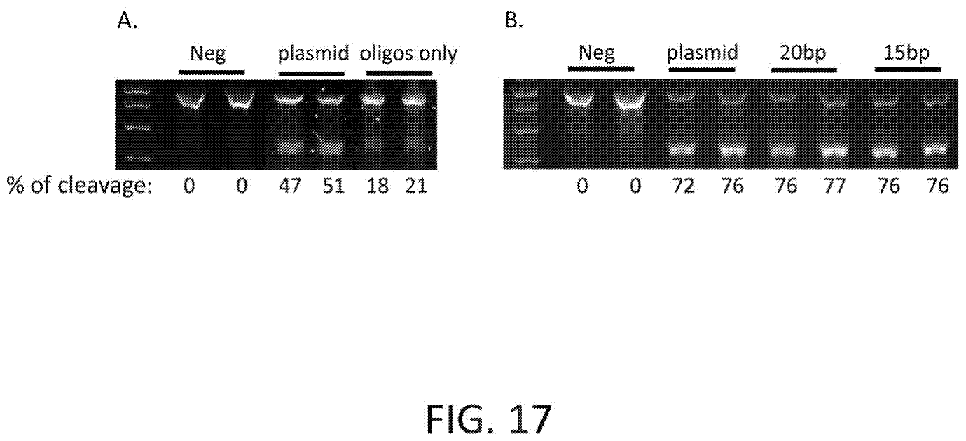

To examine in vivo functionality of synthesized gRNA, the Cas9 protein from E. coli was expressed and purified. The Cas9 protein was pre-incubated with synthetic gRNA to form the complexes prior to cell transfection. The gRNA prepared from an all-in-one plasmid served as a positive control. The genome modification was examined by Genome Cleavage and Detection assay. As depicted in Gel Image B of FIG. 17, the percentage of Indel for the newly synthesized gRNAs was similar to the positive control. To determine the error rate in the gRNA template, sequencing analysis was also performed. Approximately 7% of gRNA template harbored mutation with most deletion occurred at 3′ end and 5′ end. One mutation was detected within the target region when longer 39 bp oligonucleotides were used. The use of PAGE-purified end primers (˜20 bp each) further decreased the error rate to 3.6% with no mutation detected in the target region, which was similar to the control gRNA prepared from an all-in-one plasmid with a 2% error rate. These results indicated that the quality of gRNA were good enough for most of our applications.

Because the 80 bp tracrRNA contains a polyT at the 3′ end, there was a possibility that the Poly T had no effect on genome editing. To test this, serial deletions of PolyT at the 3′ end of gRNA (set 3 oligos in Table 7) were made. Based on in vivo genome cleavage assay, removal of the PolyT at 3′ end of gRNA appeared to have no effect on the performance of gRNA. The addition of three extra Ts at the 3′ end also did not affect the functionality of gRNA either (data not shown).

The standard T7 promoter sequence “taatacgactcactataggg” (SEQ ID NO: 11) contains GGG at the 3′ end, which is thought to be essential for maximal production of gRNA via in vitro transcription. However, because the transcription starts from the first G, three extra G will be added to the gRNA sequence assuming the target does not have a G at the 5′ end, which might affect the functionality of gRNA. To examine this, the AAVS target ccagtagccagccccgtcc (SEQ ID NO: 12) and the IP3R2 target tcgtgtccctgtacgcgga (SEQ ID NO: 13) were chosen and G deletions at the 3′ end of T7 promoter were made (see Table 6). The addition of Gs, especially 3G in a row, significantly decreased the activity of gRNA. The addition of 1 G exhibited slightly better cleavage efficiency than that of 2 G, even although both produced similar amount of gRNA in in vitro transcription reaction. However, without any G, the yield of in vitro transcription reaction was dramatically reduced.

TABLE 6
Effect of G on gRNA synthesis

| Construct | PCR yield (ng/μl) | RNA yield (ng/μl) |
|---|---|---|
| AAVS-0G | 124 | 220 |
| AAVS-1G | 82 | 394 |
| AAVS-2G | 70 | 294 |
| AAVS-3G | 53 | 290 |
| IP3R2-0G | 100 | 65 |
| IP3R2-1G | 54 | 272 |
| IP3R2-2G | 46 | 300 |
| IP3R2-3G | 122 | 289 |
| EMX1-0G | 75 | 156 |
| EMX1-1G | 119 | 490 |
| EMX1-2G | 101 | 680 |
| EMX1-3G | 98 | 750 |

In conclusion, compositions and methods provided herein related to gRNA synthesis and associated workflows allow for four day cell engineering. On Day 1, the biologists (1) design and (2) synthesize or order short DNA oligonucleotides and seed the cells of interest. On Day 2, the biologists prepare the gRNA template by one pot PCR, followed by in vitro transcription for making gRNA. Upon association of gRNA with purified Cas9 protein, the Cas9 protein/gRNA complexes are transfected into the cells via lipid-mediated method or electroporation. On Day 4, the biologists harvest the cells to analyze genome modification. Thus, the invention provides compositions and methods related to improve workflows for genome engineering. In some aspects, these workflows allow for the genome modification experiments to occur in four days from concept to completion.

TABLE 7
Oligonucleotides for gRNA synthesis

| | | SEQ ID NO |
|---|---|---|
| Set 1 oligos | | |
| gF1 | taatacgactcactataggggcatttctcagtcctaaaca | 14 |
| gR1 | GCT ATT TCT AGC TCT AAA ACT GTT TAG GAC TGA GAA ATG C | 15 |
| gF2 | gttttagagctagaaatagcaagttaaaataaggctagtccgttatcaacttgaaaaagt | 16 |
| gR2 | AAA AGC ACC GAC TCG GTG CCA CTT TTT CAA GTT GAT AAC | 17 |
| | GGA CTA GCC TTA TTT TAA CTT | |
| gEnd-F | taatacgactcactataggg | 11 |
| gEnd-R | AAA AGC ACC GAC TCG GTG CCA C | 18 |
| Set 2 oligos | | |
| Con-F | GTT TTA GAG CTA GAA ATA GCA AG | 19 |
| gEnd-R | AAA AGC ACC GAC TCG GTG CCA C | 20 |
| gR1-20 bp | GCT ATT TCT AGC TCT AAA ACT GTT TAG GAC TGA GAA ATG | 21 |
| gR1-15 bp | TTC TAG CTC TAA AAC TGT TTA GGA CTG AGA AAT G | 22 |
| gF1-20 bp | taatacgactcactataggcatttctcagtcctaaacag | 23 |
| gF1-15 bp | taatacgactcactataggcatttctcagtccta | 24 |
| gEnd-F-2G | taatacgactcactatagg | 24 |
| Set 3 oligos | | |
| gEnd-R4T | AAA AGC ACC GAC TCG GTG CCA C | 26 |
| gEnd-R3T | AA AGC ACC GAC TCG GTG CCA C | 27 |
| gEnd-R2T | A AGC ACC GAC TCG GTG CCA C | 28 |
| gEnd-R1T | AGC ACC GAC TCG GTG CCA C | 29 |
| gEnd-R0T | GC ACC GAC TCG GTG CCA C | 30 |
| Set 4 oligos | | |
| AAVS3G | taatacgactcactatagggCCAGTAGCCAGCCCC | 31 |
| AAVS2G | taatacgactcactataggCCAGTAGCCAGCCCC | 32 |
| AAVS1G | taatacgactcactatagCCAGTAGCCAGCCCC | 33 |

TABLE 8
Nucleotide sequence of a vector that may be used herein (SEQ ID NO: 34).

GTTAGGCGTTTTGCGCTGCTTCGCGATGTACGGGCCAGATATACGCGTTGACATTGATTATTGACTAGTT
ATTAATAGTAATCAATTACGGGGTCATTAGTTCATAGCCCATATATGGAGTTCCGCGTTACATAACTTAC
GGTAAATGGCCCGCCTGGCTGACCGCCCAACGACCCCCGCCCATTGACGTCAATAATGACGTATGTTCCC
ATAGTAACGCCAATAGGGACTTTCCATTGACGTCAATGGGTGGAGTATTTACGGTAAACTGCCCACTTGG
CAGTACATCAAGTGTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGTATTAGTCATCGCTA
TTACCATGGTGATGCGGTTTTGGCAGTACATCAATGGGCGTGGATAGCGGTTTGACTCACGGGGATTTCC
AAGTCTCCACCCCATTGACGTCAATGGGAGTTTGTTTTGGCACCAAAATCAACGGGACTTTCCAAAATGT
CGTAACAACTCCGCCCCATTGACGCAAATGGGCGGTAGGCGTGTACGGTGGGAGGTCTATATAAGCAGA
GCTCGTTTAGTGAACCGTCAGATCGCCTGGAGACGCCATCCACGCTGTTTTGACCTCCATAGAAGACACC
GGGACCGATCCAGCCTCCGGAGCGGCCGCCACCATGGGCAAGCCCATCCCTAACCCCCTGTTGGGGCTG
GACAGCACCGCTCCCAAAAAGAAAAGGAAGGTGGGCATTCACGGCGTGCCTGCGGCCGACAAAAAGTA
CAGCATCGGCCTTGATATCGGCACCAATAGCGTGGGCTGGGCCGTTATCACAGACGAATACAAGGTACC
CAGCAAGAAGTTCAAGGTGCTGGGGAATACAGACAGGCACTCTATCAAGAAAAACCTTATCGGGGCTCT
GCTGTTTGACTCAGGCGAGACCGCCGAGGCCACCAGGTTGAAGAGGACCGCAAGGCGAAGGTACACCC
GGAGGAAGAACAGGATCTGCTATCTGCAGGAGATCTTCAGCAACGAGATGGCCAAGGTGGACGACAGC
TTCTTCCACAGGCTGGAGGAGAGCTTCCTTGTCGAGGAGGATAAGAAGCACGAACGACACCCCATCTTC
GGCAACATAGTCGACGAGGTCGCTTATCACGAGAAGTACCCCACCATCTACCACCTGCGAAAGAAATTG
GTGGATAGCACCGATAAAGCCGACTTGCGACTTATCTACTTGGCTCTGGCGCACATGATTAAGTTCAGGG
GCCACTTCCTGATCGAGGGCGACCTTAACCCCGACAACAGTGACGTAGACAAATTGTTCATCCAGCTTGT
ACAGACCTATAACCAGCTGTTCGAGGAAAACCCTATTAACGCCAGCGGGGTGGATGCGAAGGCCATACT
TAGCGCCAGGCTGAGCAAAAGCAGGCGCTTGGAGAACCTGATAGCCCAGCTGCCCGGTGAAAAGAAGA
ACGGCCTCTTCGGTAATCTGATTGCCCTGAGCCTGGGCCTGACCCCCAACTTCAAGAGCAACTTCGACCT
GGCAGAAGATGCCAAGCTGCAGTTGAGTAAGGACACCTATGACGACGACTTGGACAATCTGCTCGCCCA
AATCGGCGACCAGTACGCTGACCTGTTCCTCGCCGCCAAGAACCTTTCTGACGCAATCCTGCTTAGCGAT
ATCCTTAGGGTGAACACAGAGATCACCAAGGCCCCCCTGAGCGCCAGCATGATCAAGAGGTACGACGAG
CACCATCAGGACCTGACCCTTCTGAAGGCCCTGGTGAGGCAGCAACTGCCCGAGAAGTACAAGGAGATC
TTTTTCGACCAGAGCAAGAACGGCTACGCCGGCTACATCGACGGCGGAGCCAGCCAAGAGGAGTTCTAC
AAGTTCATCAAGCCCATCCTGGAGAAGATGGATGGCACCGAGGAGCTGCTGGTGAAGCTGAACAGGGA
AGATTTGCTCCGGAAGCAGAGGACCTTTGACAACGGTAGCATCCCCCACCAGATCCACCTGGGCGAGCT
GCACGCAATACTGAGGCGACAGGAGGATTTCTACCCCTTCCTCAAGGACAATAGGGAGAAAATCGAAAA
GATTCTGACCTTCAGGATCCCCTACTACGTGGGCCCTCTTGCCAGGGGCAACAGCCGATTCGCTTGGATG
ACAAGAAAGAGCGAGGAGACCATCACCCCCTGGAACTTCGAGGAAGTGGTGGACAAAGGAGCAAGCGC
GCAGTCTTTCATCGAACGGATGACCAATTTCGACAAAAACCTGCCTAACGAGAAGGTGCTGCCCAAGCA
CAGCCTGCTTTACGAGTACTTCACCGTGTACAACGAGCTCACCAAGGTGAAATATGTGACCGAGGGCAT
GCGAAAACCCGCTTTCCTGAGCGGCGAGCAGAAGAAGGCCATCGTGGACCTGCTGTTCAAGACCAACAG
GAAGGTGACCGTGAAGCAGCTGAAGGAGGACTACTTCAAGAAGATCGAGTGCTTTGATAGCGTGGAAAT
AAGCGGCGTGGAGGACAGGTTCAACGCCAGCCTGGGCACCTACCACGACTTGTTGAAGATAATCAAAGA
CAAGGATTTCCTGGATAATGAGGAGAACGAGGATATACTCGAGGACATCGTGCTGACTTTGACCCTGTTT
GAGGACCGAGAGATGATTGAAGAAAGGCTCAAAACCTACGCCCACCTGTTCGACGACAAAGTGATGAA
ACAACTGAAGAGACGAAGATACACCGGCTGGGGCAGACTGTCCAGGAAGCTCATCAACGGCATTAGGG
ACAAGCAGAGCGGCAAGACCATCCTGGATTTCCTGAAGTCCGACGGCTTCGCCAACCGAAACTTCATGC
AGCTGATTCACGATGACAGCTTGACCTTCAAGGAGGACATCCAGAAGGCCCAGGTTAGCGGCCAGGGCG
ACTCCCTGCACGAACATATTGCAAACCTGGCAGGCTCCCCTGCGATCAAGAAGGGCATACTGCAGACCG
TTAAGGTTGTGGACGAATTGGTCAAGGTCATGGGCAGGCACAAGCCCGAAAACATAGTTATAGAGATGG
CCAGAGAGAACCAGACCACCCAAAAGGGCCAGAAGAACAGCCGGGAGCGCATGAAAAGGATCGAGGA
GGGTATCAAGGAACTCGGAAGCCAGATCCTCAAAGAGCACCCCGTGGAGAATACCCAGCTCCAGAACG
AGAAGCTGTACCTGTACTACCTGCAGAACGGCAGGGACATGTACGTTGACCAGGAGTTGGACATCAACA
GGCTTTCAGACTATGACGTGGATCACATAGTGCCCCAGAGCTTTCTTAAAGACGATAGCATCGACAACA
AGGTCCTGACCCGCTCCGACAAAAACAGGGGCAAAAGCGACAACGTGCCAAGCGAAGAGGTGGTTAAA
AAGATGAAGAACTACTGGAGGCAACTGCTCAACGCGAAATTGATCACCCAGAGAAAGTTCGATAACCTG
ACCAAGGCCGAGAGGGGCGGACTCTCCGAACTTGACAAAGCGGGCTTCATAAAGAGGCAGCTGGTCGA
GACCCGACAGATCACGAAGCACGTGGCCCAAATCCTCGACAGCAGAATGAATACCAAGTACGATGAGA
ATGACAAACTCATCAGGGAAGTGAAAGTGATTACCCTGAAGAGCAAGTTGGTGTCCGACTTTCGCAAAG
ATTTCCAGTTCTACAAGGTGAGGGAGATCAACAACTACCACCATGCCCACGACGCATACCTGAACGCCG
TGGTCGGCACCGCCCTGATTAAGAAGTATCCAAAGCTGGAGTCCGAATTTGTCTACGGCGACTACAAAG
TTTACGATGTGAGGAAGATGATCGCTAAGAGCGAACAGGAGATCGGCAAGGCCACCGCTAAGTATTTCT
TCTACAGCAACATCATGAACTTTTTCAAGACCGAGATCACACTTGCCAACGGCGAAATCAGGAAGAGGC
CGCTTATCGAGACCAACGGTGAGACCGGCGAGATCGTGTGGGACAAGGGCAGGGACTTCGCCACCGTGA
GGAAAGTCCTGAGCATGCCCCAGGTGAATATTGTGAAAAAAACTGAGGTGCAGACAGGCGGCTTTAGCA
AGGAATCCATCCTGCCCAAGAGGAACAGCGACAAGCTGATCGCCCGGAAGAAGGACTGGGACCCTAAG
AAGTATGGAGGCTTCGACAGCCCCACCGTAGCCTACAGCGTGCTGGTGGTCGCGAAGGTAGAGAAGGGG
AAGAGCAAGAAACTGAAGAGCGTGAAGGAGCTGCTCGGCATAACCATCATGGAGAGGTCCAGCTTTGA
GAAGAACCCCATTGACTTTTTGGAAGCCAAGGGCTACAAAGAGGTCAAAAAGGACCTGATCATCAAACT
CCCCAAGTACTCCCTGTTTGAATTGGAGAACGGCAGAAAGAGGATGCTGGCGAGCGCTGGGGAACTGCA
AAAGGGCAACGAACTGGCGCTGCCCAGCAAGTACGTGAATTTTCTGTACCTGGCGTCCCACTACGAAAA
GCTGAAAGGCAGCCCCGAGGACAACGAGCAGAAGCAGCTGTTCGTGGAGCAGCACAAGCATTACCTGG
ACGAGATAATCGAGCAAATCAGCGAGTTCAGCAAGAGGGTGATTCTGGCCGACGCGAACCTGGATAAG
GTCCTCAGCGCCTACAACAAGCACCGAGACAAACCCATCAGGGAGCAGGCCGAGAATATCATACACCTG
TTCACCCTGACAAATCTGGGCGCACCTGCGGCATTCAAATACTTCGATACCACCATCGACAGGAAAAGG
TACACTAGCACTAAGGAGGTGCTGGATGCCACCTTGATCCACCAGTCCATTACCGGCCTGTATGAGACCA
GGATCGACCTGAGCCAGCTTGGAGGCGACTCTAGGGCGGACCCAAAAAAGAAAAGGAAGGTGGAATTC
TCTAGAGGCAGTGGAGAGGGCAGAGGAAGTCTGCTAACATGCGGTGACGTCGAGGAGAATCCTGGCCC
AATGAACCGGGGAGTCCCTTTTAGGCACTTGCTTCTGGTGCTGCAACTGGCGCTCCTCCCAGCAGCCACT
CAGGGAAAGAAAGTGGTGCTGGGCAAAAAAGGGGATACAGTGGAACTGACCTGTACAGCTTCCCAGAA
GAAGAGCATACAATTCCACTGGAAAAACTCCAACCAGATAAAGATTCTGGGAAATCAGGGCTCCTTCTT
AACTAAAGGTCCATCCAAGCTGAATGATCGCGCTGACTCAAGAAGAAGCCTTTGGGACCAAGGAAACTT
CCCCCTGATCATCAAGAATCTTAAGATAGAAGACTCAGATACTTACATCTGTGAAGTGGAGGACCAGAA
GGAGGAGGTGCAATTGCTAGTGTTCGGATTGACTGCCAACTCTGACACCCACCTGCTTCAGGGGCAGAG
CCTGACCCTGACCTTGGAGAGCCCCCCTGGTAGTAGCCCCTCAGTGCAATGTAGGAGTCCAAGGGGTAA
AAACATACAGGGGGGGAAGACCCTCTCCGTGTCTCAGCTGGAGCTCCAGGATAGTGGCACCTGGACATG
CACTGTCTTGCAGAACCAGAAGAAGGTGGAGTTCAAAATAGACATCGTGGTGCTAGCTTTCCAGAAGGC
CTCCAGCATAGTCTATAAGAAAGAGGGGGAACAGGTGGAGTTCTCCTTCCCACTCGCCTTTACAGTTGAA
AAGCTGACGGGCAGTGGCGAGCTGTGGTGGCAGGCGGAGAGGGCTTCCTCCTCCAAGTCTTGGATCACC
TTTGACCTGAAGAACAAGGAAGTGTCTGTAAAACGGGTTACCCAGGACCCTAAGCTCCAGATGGGCAAG
AAGCTCCCGCTCCACCTCACCCTGCCCCAGGCCTTGCCTCAGTATGCTGGCTCTGGAAACCTCACCCTGG
CCCTTGAAGCGAAAACAGGAAAGTTGCATCAGGAAGTGAACCTGGTGGTGATGAGAGCCACTCAGCTCC
AGAAAAATTTGACCTGTGAGGTGTGGGGACCCACCTCCCCTAAGCTGATGCTGAGCTTGAAACTGGAGA
ACAAGGAGGCAAAGGTCTCGAAGCGGGAGAAGGCGGTGTGGGTGCTGAACCCTGAGGCGGGGATGTGG
CAGTGTCTGCTGAGTGACTCGGGACAGGTCCTGCTGGAATCCAACATCAAGGTTCTGCCCACATGGTCGA
CCCCGGTGCAGCCAATGGCCCTGATTGTGCTGGGGGGCGTCGCCGGCCTCCTGCTTTTCATTGGGCTAGG
CATCTTCTTCTGTGTCAGGTGCCGGCACACCGGTTAGTAATGAGTTTAAACGGGGGAGGCTAACTGAAAC
ACGGAAGGAGACAATACCGGAAGGAACCCGCGCTATGACGGCAATAAAAAGACAGAATAAAACGCACG
GGTGTTGGGTCGTTTGTTCATAAACGCGGGGTTCGGTCCCAGGGCTGGCACTCTGTCGATACCCCACCGA
GACCCCATTGGGGCCAATACGCCCGCGTTTCTTCCTTTTCCCCACCCCACCCCCCAAGTTCGGGTGAAGG
CCCAGGGCTCGCAGCCAACGTCGGGGCGGCAGGCCCTGCCATAGCAGATCTGCGCAGCTGGGGCTCTAG
GGGGTATCCCCACGCGCCCTGTAGCGGCGCATTAAGCGCGGCGGGTGTGGTGGTTACGCGCAGCGTGAC
CGCTACACTTGCCAGCGCCCTAGCGCCCGCTCCTTTCGCTTTCTTCCCTTCCTTTCTCGCCACGTTCGCCG
GCTTTCCCCGTCAAGCTCTAAATCGGGGGCTCCCTTTAGGGTTCCGATTTAGTGCTTTACGGCACCTCGAC
CCCAAAAAACTTGATTAGGGTGATGGTTCACGTAGTGGGCCATCGCCCTGATAGACGGTTTTTCGCCCTT
TGACGTTGGAGTCCACGTTCTTTAATAGTGGACTCTTGTTCCAAACTGGAACAACACTCAACCCTATCTC
GGTCTATTCTTTTGATTTATAAGGGATTTTGCCGATTTCGGCCTATTGGTTAAAAAATGAGCTGATTTAAC
AAAAATTTAACGCGAATTAATTAAGGTCGGGCAGGAAGAGGGCCTATTTCCCATGATTCCTTCATATTTG
CATATACGATACAAGGCTGTTAGAGAGATAATTAGAATTAATTTGACTGTAAACACAAAGATATTAGTA
CAAAATACGTGACGTAGAAAGTAATAATTTCTTGGGTAGTTTGCAGTTTTAAAATTATGTTTTAAAATGG
ACTATCATATGCTTACCGTAACTTGAAAGTATTTCGATTTCTTGGCTTTATATATCTTGTGGAAAGGACGA
AACACCGNNNNNNNNNNNNNNNNNNNNGTTTTAGAGCTAGAAATAGCAAGTTAAAATAAGGCTAGTCC
GTTATCAACTTGAAAAAGTGGCACCGAGTCGGTGCTTTTTTCTAGTATACCGTCGACCTCTAGCTAGAGC
TTGGCGTAATCATGGTCATAGCTGTTTCCTGTGTGAAATTGTTATCCGCTCACAATTCCACACAACATAC
GAGCCGGAAGCATAAAGTGTAAAGCCTGGGGTGCCTAATGAGTGAGCTAACTCACATTAATTGCGTTGC
GCTCACTGCCCGCTTTCCAGTCGGGAAACCTGTCGTGCCAGCTGCATTAATGAATCGGCCAACGCGCGGG
GAGAGGCGGTTTGCGTATTGGGCGCTCTTCCGCTTCCTCGCTCACTGACTCGCTGCGCTCGGTCGTTCGG
CTGCGGCGAGCGGTATCAGCTCACTCAAAGGCGGTAATACGGTTATCCACAGAATCAGGGGATAACGCA
GGAAAGAACATGTGAGCAAAAGGCCAGCAAAAGGCCAGGAACCGTAAAAAGGCCGCGTTGCTGGCGTT
TTTCCATAGGCTCCGCCCCCCTGACGAGCATCACAAAAATCGACGCTCAAGTCAGAGGTGGCGAAACCC
GACAGGACTATAAAGATACCAGGCGTTTCCCCCTGGAAGCTCCCTCGTGCGCTCTCCTGTTCCGACCCTG
CCGCTTACCGGATACCTGTCCGCCTTTCTCCCTTCGGGAAGCGTGGCGCTTTCTCATAGCTCACGCTGTAG
GTATCTCAGTTCGGTGTAGGTCGTTCGCTCCAAGCTGGGCTGTGTGCACGAACCCCCCGTTCAGCCCGAC
CGCTGCGCCTTATCCGGTAACTATCGTCTTGAGTCCAACCCGGTAAGACACGACTTATCGCCACTGGCAG
CAGCCACTGGTAACAGGATTAGCAGAGCGAGGTATGTAGGCGGTGCTACAGAGTTCTTGAAGTGGTGGC
CTAACTACGGCTACACTAGAAGAACAGTATTTGGTATCTGCGCTCTGCTGAAGCCAGTTACCTTCGGAAA
AAGAGTTGGTAGCTCTTGATCCGGCAAACAAACCACCGCTGGTAGCGGTGGTTTTTTTGTTTGCAAGCAG
CAGATTACGCGCAGAAAAAAAGGATCTCAAGAAGATCCTTTGATCTTTTCTACGGGGTCTGACGCTCAGT
GGAACGAAAACTCACGTTAAGGGATTTTGGTCATGAGATTATCAAAAAGGATCTTCACCTAGATCCTTTT
AAATTAAAAATGAAGTTTTAAATCAATCTAAAGTATATATGAGTAAACTTGGTCTGACAGTTACCAATGC
TTAATCAGTGAGGCACCTATCTCAGCGATCTGTCTATTTCGTTCATCCATAGTTGCCTGACTCCCCGTCGT
GTAGATAACTACGATACGGGAGGGCTTACCATCTGGCCCCAGTGCTGCAATGATACCGCGAGACCCACG
CTCACCGGCTCCAGATTTATCAGCAATAAACCAGCCAGCCGGAAGGGCCGAGCGCAGAAGTGGTCCTGC
AACTTTATCCGCCTCCATCCAGTCTATTAATTGTTGCCGGGAAGCTAGAGTAAGTAGTTCGCCAGTTAAT
AGTTTGCGCAACGTTGTTGCCATTGCTACAGGCATCGTGGTGTCACGCTCGTCGTTTGGTATGGCTTCATT
CAGCTCCGGTTCCCAACGATCAAGGCGAGTTACATGATCCCCCATGTTGTGCAAAAAAGCGGTTAGCTCC
TTCGGTCCTCCGATCGTTGTCAGAAGTAAGTTGGCCGCAGTGTTATCACTCATGGTTATGGCAGCACTGC
ATAATTCTCTTACTGTCATGCCATCCGTAAGATGCTTTTCTGTGACTGGTGAGTACTCAACCAAGTCATTC
TGAGAATAGTGTATGCGGCGACCGAGTTGCTCTTGCCCGGCGTCAATACGGGATAATACCGCGCCACAT
AGCAGAACTTTAAAAGTGCTCATCATTGGAAAACGTTCTTCGGGGCGAAAACTCTCAAGGATCTTACCG
CTGTTGAGATCCAGTTCGATGTAACCCACTCGTGCACCCAACTGATCTTCAGCATCTTTTACTTTCACCAG
CGTTTCTGGGTGAGCAAAAACAGGAAGGCAAAATGCCGCAAAAAAGGGAATAAGGGCGACACGGAAAT
GTTGAATACTCATACTCTTCCTTTTTCAATATTATTGAAGCATTTATCAGGGTTATTGTCTCATGAGCGGA
TACATATTTGAATGTATTTAGAAAAATAAACAAATAGGGGTTCCGCGCACATTTCCCCGAAAAGTGCCA
CCTGACGTCGACGGATCGGGAGATCTCCCGATCCCCTATGGTGCACTCTCAGTACAATCTGCTCTGATGC
CGCATAGTTAAGCCAGTATCTGCTCCCTGCTTGTGTGTTGGAGGTCGCTGAGTAGTGCGCGAGCAAAATT
TAAGCTACAACAAGGCAAGGCTTGACCGACAATTGCATGAAGAATCTGCTTAGG

TABLE 9
Nucleotide sequence of a vector that may be used herein (SEQ ID NO: 35)

GTTAGGCGTTTTGCGCTGCTTCGCGATGTACGGGCCAGATATACGCGTTGACATTGATTATTGACTAGTT
ATTAATAGTAATCAATTACGGGGTCATTAGTTCATAGCCCATATATGGAGTTCCGCGTTACATAACTTAC
GGTAAATGGCCCGCCTGGCTGACCGCCCAACGACCCCCGCCCATTGACGTCAATAATGACGTATGTTCCC
ATAGTAACGCCAATAGGGACTTTCCATTGACGTCAATGGGTGGAGTATTTACGGTAAACTGCCCACTTGG
CAGTACATCAAGTGTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGTATTAGTCATCGCTA
TTACCATGGTGATGCGGTTTTGGCAGTACATCAATGGGCGTGGATAGCGGTTTGACTCACGGGGATTTCC
AAGTCTCCACCCCATTGACGTCAATGGGAGTTTGTTTTGGCACCAAAATCAACGGGACTTTCCAAAATGT
CGTAACAACTCCGCCCCATTGACGCAAATGGGCGGTAGGCGTGTACGGTGGGAGGTCTATATAAGCAGA
GCTCGTTTAGTGAACCGTCAGATCGCCTGGAGACGCCATCCACGCTGTTTTGACCTCCATAGAAGACACC
GGGACCGATCCAGCCTCCGGAGCGGCCGCCACCATGGGCAAGCCCATCCCTAACCCCCTGTTGGGGCTG
GACAGCACCGCTCCCAAAAAGAAAAGGAAGGTGGGCATTCACGGCGTGCCTGCGGCCGACAAAAAGTA
CAGCATCGGCCTTGATATCGGCACCAATAGCGTGGGCTGGGCCGTTATCACAGACGAATACAAGGTACC
CAGCAAGAAGTTCAAGGTGCTGGGGAATACAGACAGGCACTCTATCAAGAAAAACCTTATCGGGGCTCT
GCTGTTTGACTCAGGCGAGACCGCCGAGGCCACCAGGTTGAAGAGGACCGCAAGGCGAAGGTACACCC
GGAGGAAGAACAGGATCTGCTATCTGCAGGAGATCTTCAGCAACGAGATGGCCAAGGTGGACGACAGC
TTCTTCCACAGGCTGGAGGAGAGCTTCCTTGTCGAGGAGGATAAGAAGCACGAACGACACCCCATCTTC
GGCAACATAGTCGACGAGGTCGCTTATCACGAGAAGTACCCCACCATCTACCACCTGCGAAAGAAATTG
GTGGATAGCACCGATAAAGCCGACTTGCGACTTATCTACTTGGCTCTGGCGCACATGATTAAGTTCAGGG
GCCACTTCCTGATCGAGGGCGACCTTAACCCCGACAACAGTGACGTAGACAAATTGTTCATCCAGCTTGT
ACAGACCTATAACCAGCTGTTCGAGGAAAACCCTATTAACGCCAGCGGGGTGGATGCGAAGGCCATACT
TAGCGCCAGGCTGAGCAAAAGCAGGCGCTTGGAGAACCTGATAGCCCAGCTGCCCGGTGAAAAGAAGA
ACGGCCTCTTCGGTAATCTGATTGCCCTGAGCCTGGGCCTGACCCCCAACTTCAAGAGCAACTTCGACCT
GGCAGAAGATGCCAAGCTGCAGTTGAGTAAGGACACCTATGACGACGACTTGGACAATCTGCTCGCCCA
AATCGGCGACCAGTACGCTGACCTGTTCCTCGCCGCCAAGAACCTTTCTGACGCAATCCTGCTTAGCGAT
ATCCTTAGGGTGAACACAGAGATCACCAAGGCCCCCCTGAGCGCCAGCATGATCAAGAGGTACGACGAG
CACCATCAGGACCTGACCCTTCTGAAGGCCCTGGTGAGGCAGCAACTGCCCGAGAAGTACAAGGAGATC
TTTTTCGACCAGAGCAAGAACGGCTACGCCGGCTACATCGACGGCGGAGCCAGCCAAGAGGAGTTCTAC
AAGTTCATCAAGCCCATCCTGGAGAAGATGGATGGCACCGAGGAGCTGCTGGTGAAGCTGAACAGGGA
AGATTTGCTCCGGAAGCAGAGGACCTTTGACAACGGTAGCATCCCCCACCAGATCCACCTGGGCGAGCT
GCACGCAATACTGAGGCGACAGGAGGATTTCTACCCCTTCCTCAAGGACAATAGGGAGAAAATCGAAAA
GATTCTGACCTTCAGGATCCCCTACTACGTGGGCCCTCTTGCCAGGGGCAACAGCCGATTCGCTTGGATG
ACAAGAAAGAGCGAGGAGACCATCACCCCCTGGAACTTCGAGGAAGTGGTGGACAAAGGAGCAAGCGC
GCAGTCTTTCATCGAACGGATGACCAATTTCGACAAAAACCTGCCTAACGAGAAGGTGCTGCCCAAGCA
CAGCCTGCTTTACGAGTACTTCACCGTGTACAACGAGCTCACCAAGGTGAAATATGTGACCGAGGGCAT
GCGAAAACCCGCTTTCCTGAGCGGCGAGCAGAAGAAGGCCATCGTGGACCTGCTGTTCAAGACCAACAG
GAAGGTGACCGTGAAGCAGCTGAAGGAGGACTACTTCAAGAAGATCGAGTGCTTTGATAGCGTGGAAAT
AAGCGGCGTGGAGGACAGGTTCAACGCCAGCCTGGGCACCTACCACGACTTGTTGAAGATAATCAAAGA
CAAGGATTTCCTGGATAATGAGGAGAACGAGGATATACTCGAGGACATCGTGCTGACTTTGACCCTGTTT
GAGGACCGAGAGATGATTGAAGAAAGGCTCAAAACCTACGCCCACCTGTTCGACGACAAAGTGATGAA
ACAACTGAAGAGACGAAGATACACCGGCTGGGGCAGACTGTCCAGGAAGCTCATCAACGGCATTAGGG
ACAAGCAGAGCGGCAAGACCATCCTGGATTTCCTGAAGTCCGACGGCTTCGCCAACCGAAACTTCATGC
AGCTGATTCACGATGACAGCTTGACCTTCAAGGAGGACATCCAGAAGGCCCAGGTTAGCGGCCAGGGCG
ACTCCCTGCACGAACATATTGCAAACCTGGCAGGCTCCCCTGCGATCAAGAAGGGCATACTGCAGACCG
TTAAGGTTGTGGACGAATTGGTCAAGGTCATGGGCAGGCACAAGCCCGAAAACATAGTTATAGAGATGG
CCAGAGAGAACCAGACCACCCAAAAGGGCCAGAAGAACAGCCGGGAGCGCATGAAAAGGATCGAGGA
GGGTATCAAGGAACTCGGAAGCCAGATCCTCAAAGAGCACCCCGTGGAGAATACCCAGCTCCAGAACG
AGAAGCTGTACCTGTACTACCTGCAGAACGGCAGGGACATGTACGTTGACCAGGAGTTGGACATCAACA
GGCTTTCAGACTATGACGTGGATCACATAGTGCCCCAGAGCTTTCTTAAAGACGATAGCATCGACAACA
AGGTCCTGACCCGCTCCGACAAAAACAGGGGCAAAAGCGACAACGTGCCAAGCGAAGAGGTGGTTAAA
AAGATGAAGAACTACTGGAGGCAACTGCTCAACGCGAAATTGATCACCCAGAGAAAGTTCGATAACCTG
ACCAAGGCCGAGAGGGGCGGACTCTCCGAACTTGACAAAGCGGGCTTCATAAAGAGGCAGCTGGTCGA
GACCCGACAGATCACGAAGCACGTGGCCCAAATCCTCGACAGCAGAATGAATACCAAGTACGATGAGA
ATGACAAACTCATCAGGGAAGTGAAAGTGATTACCCTGAAGAGCAAGTTGGTGTCCGACTTTCGCAAAG
ATTTCCAGTTCTACAAGGTGAGGGAGATCAACAACTACCACCATGCCCACGACGCATACCTGAACGCCG
TGGTCGGCACCGCCCTGATTAAGAAGTATCCAAAGCTGGAGTCCGAATTTGTCTACGGCGACTACAAAG
TTTACGATGTGAGGAAGATGATCGCTAAGAGCGAACAGGAGATCGGCAAGGCCACCGCTAAGTATTTCT
TCTACAGCAACATCATGAACTTTTTCAAGACCGAGATCACACTTGCCAACGGCGAAATCAGGAAGAGGC
CGCTTATCGAGACCAACGGTGAGACCGGCGAGATCGTGTGGGACAAGGGCAGGGACTTCGCCACCGTGA
GGAAAGTCCTGAGCATGCCCCAGGTGAATATTGTGAAAAAAACTGAGGTGCAGACAGGCGGCTTTAGCA
AGGAATCCATCCTGCCCAAGAGGAACAGCGACAAGCTGATCGCCCGGAAGAAGGACTGGGACCCTAAG
AAGTATGGAGGCTTCGACAGCCCCACCGTAGCCTACAGCGTGCTGGTGGTCGCGAAGGTAGAGAAGGGG
AAGAGCAAGAAACTGAAGAGCGTGAAGGAGCTGCTCGGCATAACCATCATGGAGAGGTCCAGCTTTGA
GAAGAACCCCATTGACTTTTTGGAAGCCAAGGGCTACAAAGAGGTCAAAAAGGACCTGATCATCAAACT
CCCCAAGTACTCCCTGTTTGAATTGGAGAACGGCAGAAAGAGGATGCTGGCGAGCGCTGGGGAACTGCA
AAAGGGCAACGAACTGGCGCTGCCCAGCAAGTACGTGAATTTTCTGTACCTGGCGTCCCACTACGAAAA
GCTGAAAGGCAGCCCCGAGGACAACGAGCAGAAGCAGCTGTTCGTGGAGCAGCACAAGCATTACCTGG
ACGAGATAATCGAGCAAATCAGCGAGTTCAGCAAGAGGGTGATTCTGGCCGACGCGAACCTGGATAAG
GTCCTCAGCGCCTACAACAAGCACCGAGACAAACCCATCAGGGAGCAGGCCGAGAATATCATACACCTG
TTCACCCTGACAAATCTGGGCGCACCTGCGGCATTCAAATACTTCGATACCACCATCGACAGGAAAAGG
TACACTAGCACTAAGGAGGTGCTGGATGCCACCTTGATCCACCAGTCCATTACCGGCCTGTATGAGACCA
GGATCGACCTGAGCCAGCTTGGAGGCGACTCTAGGGCGGACCCAAAAAAGAAAAGGAAGGTGGAATTC
TCTAGAGGCAGTGGAGAGGGCAGAGGAAGTCTGCTAACATGCGGTGACGTCGAGGAGAATCCTGGCCC
AATGAACCTGAGCAAAAACGTGAGCGTGAGCGTGTATATGAAGGGGAACGTCAACAATCATGAGTTTGA
GTACGACGGGGAAGGTGGTGGTGATCCTTATACAGGTAAATATTCCATGAAGATGACGCTACGTGGTCA
AAATTCCCTACCCTTTTCCTATGATATCATTACCACGGCATTTCAGTATGGTTTCCGCGTATTTACAAAAT
ACCCTGAGGGAATTGTTGACTATTTTAAGGACTCGCTTCCCGACGCATTCCAGTGGAACAGACGAATTGT
GTTTGAAGATGGTGGAGTACTAAACATGAGCAGTGATATCACATATAAAGATAATGTTCTGCATGGTGA
CGTCAAGGCTGAGGGAGTGAACTTCCCGCCGAATGGGCCAGTGATGAAGAATGAAATTGTGATGGAGG
AACCGACTGAAGAAACATTTACTCCAAAAAACGGGGTTCTTGTTGGCTTTTGTCCCAAAGCGTACTTACT
TAAAGACGGTTCCTATTACTATGGAAATATGACAACATTTTACAGATCCAAGAAATCTGGCCAGGCACCT
CCTGGGTATCACTTTGTTAAGCATCGTCTCGTCAAGACCAATGTGGGACATGGATTTAAGACGGTTGAGC
AGACTGAATATGCCACTGCTCATGTCAGTGATCTTCCCAAGTTCGAAGCTTGATAATGAGTTTAAACGGG
GGAGGCTAACTGAAACACGGAAGGAGACAATACCGGAAGGAACCCGCGCTATGACGGCAATAAAAAGA
CAGAATAAAACGCACGGGTGTTGGGTCGTTTGTTCATAAACGCGGGGTTCGGTCCCAGGGCTGGCACTC
TGTCGATACCCCACCGAGACCCCATTGGGGCCAATACGCCCGCGTTTCTTCCTTTTCCCCACCCCACCCCC
CAAGTTCGGGTGAAGGCCCAGGGCTCGCAGCCAACGTCGGGGCGGCAGGCCCTGCCATAGCAGATCTGC
GCAGCTGGGGCTCTAGGGGGTATCCCCACGCGCCCTGTAGCGGCGCATTAAGCGCGGCGGGTGTGGTGG
TTACGCGCAGCGTGACCGCTACACTTGCCAGCGCCCTAGCGCCCGCTCCTTTCGCTTTCTTCCCTTCCTTT
CTCGCCACGTTCGCCGGCTTTCCCCGTCAAGCTCTAAATCGGGGGCTCCCTTTAGGGTTCCGATTTAGTGC
TTTACGGCACCTCGACCCCAAAAAACTTGATTAGGGTGATGGTTCACGTAGTGGGCCATCGCCCTGATAG
ACGGTTTTTCGCCCTTTGACGTTGGAGTCCACGTTCTTTAATAGTGGACTCTTGTTCCAAACTGGAACAAC
ACTCAACCCTATCTCGGTCTATTCTTTTGATTTATAAGGGATTTTGCCGATTTCGGCCTATTGGTTAAAAA
ATGAGCTGATTTAACAAAAATTTAACGCGAATTAATTAAGGTCGGGCAGGAAGAGGGCCTATTTCCCAT
GATTCCTTCATATTTGCATATACGATACAAGGCTGTTAGAGAGATAATTAGAATTAATTTGACTGTAAAC
ACAAAGATATTAGTACAAAATACGTGACGTAGAAAGTAATAATTTCTTGGGTAGTTTGCAGTTTTAAAAT
TATGTTTTAAAATGGACTATCATATGCTTACCGTAACTTGAAAGTATTTCGATTTCTTGGCTTTATATATC
TTGTGGAAAGGACGAAACACCGNNNNNNNNNNNNNNNNNNNNGTTTTAGAGCTAGAAATAGCAAGTTA
AAATAAGGCTAGTCCGTTATCAACTTGAAAAAGTGGCACCGAGTCGGTGCTTTTTTCTAGTATACCGTCG
ACCTCTAGCTAGAGCTTGGCGTAATCATGGTCATAGCTGTTTCCTGTGTGAAATTGTTATCCGCTCACAAT
TCCACACAACATACGAGCCGGAAGCATAAAGTGTAAAGCCTGGGGTGCCTAATGAGTGAGCTAACTCAC
ATTAATTGCGTTGCGCTCACTGCCCGCTTTCCAGTCGGGAAACCTGTCGTGCCAGCTGCATTAATGAATC
GGCCAACGCGCGGGGAGAGGCGGTTTGCGTATTGGGCGCTCTTCCGCTTCCTCGCTCACTGACTCGCTGC
GCTCGGTCGTTCGGCTGCGGCGAGCGGTATCAGCTCACTCAAAGGCGGTAATACGGTTATCCACAGAAT
CAGGGGATAACGCAGGAAAGAACATGTGAGCAAAAGGCCAGCAAAAGGCCAGGAACCGTAAAAAGGC
CGCGTTGCTGGCGTTTTTCCATAGGCTCCGCCCCCCTGACGAGCATCACAAAAATCGACGCTCAAGTCAG
AGGTGGCGAAACCCGACAGGACTATAAAGATACCAGGCGTTTCCCCCTGGAAGCTCCCTCGTGCGCTCT
CCTGTTCCGACCCTGCCGCTTACCGGATACCTGTCCGCCTTTCTCCCTTCGGGAAGCGTGGCGCTTTCTCA
TAGCTCACGCTGTAGGTATCTCAGTTCGGTGTAGGTCGTTCGCTCCAAGCTGGGCTGTGTGCACGAACCC
CCCGTTCAGCCCGACCGCTGCGCCTTATCCGGTAACTATCGTCTTGAGTCCAACCCGGTAAGACACGACT
TATCGCCACTGGCAGCAGCCACTGGTAACAGGATTAGCAGAGCGAGGTATGTAGGCGGTGCTACAGAGT
TCTTGAAGTGGTGGCCTAACTACGGCTACACTAGAAGAACAGTATTTGGTATCTGCGCTCTGCTGAAGCC
AGTTACCTTCGGAAAAAGAGTTGGTAGCTCTTGATCCGGCAAACAAACCACCGCTGGTAGCGGTGGTTTT
TTTGTTTGCAAGCAGCAGATTACGCGCAGAAAAAAAGGATCTCAAGAAGATCCTTTGATCTTTTCTACGG
GGTCTGACGCTCAGTGGAACGAAAACTCACGTTAAGGGATTTTGGTCATGAGATTATCAAAAAGGATCT
TCACCTAGATCCTTTTAAATTAAAAATGAAGTTTTAAATCAATCTAAAGTATATATGAGTAAACTTGGTC
TGACAGTTACCAATGCTTAATCAGTGAGGCACCTATCTCAGCGATCTGTCTATTTCGTTCATCCATAGTTG
CCTGACTCCCCGTCGTGTAGATAACTACGATACGGGAGGGCTTACCATCTGGCCCCAGTGCTGCAATGAT
ACCGCGAGACCCACGCTCACCGGCTCCAGATTTATCAGCAATAAACCAGCCAGCCGGAAGGGCCGAGCG
CAGAAGTGGTCCTGCAACTTTATCCGCCTCCATCCAGTCTATTAATTGTTGCCGGGAAGCTAGAGTAAGT
AGTTCGCCAGTTAATAGTTTGCGCAACGTTGTTGCCATTGCTACAGGCATCGTGGTGTCACGCTCGTCGT
TTGGTATGGCTTCATTCAGCTCCGGTTCCCAACGATCAAGGCGAGTTACATGATCCCCCATGTTGTGCAA
AAAAGCGGTTAGCTCCTTCGGTCCTCCGATCGTTGTCAGAAGTAAGTTGGCCGCAGTGTTATCACTCATG
GTTATGGCAGCACTGCATAATTCTCTTACTGTCATGCCATCCGTAAGATGCTTTTCTGTGACTGGTGAGTA
CTCAACCAAGTCATTCTGAGAATAGTGTATGCGGCGACCGAGTTGCTCTTGCCCGGCGTCAATACGGGAT
AATACCGCGCCACATAGCAGAACTTTAAAAGTGCTCATCATTGGAAAACGTTCTTCGGGGCGAAAACTC
TCAAGGATCTTACCGCTGTTGAGATCCAGTTCGATGTAACCCACTCGTGCACCCAACTGATCTTCAGCAT
CTTTTACTTTCACCAGCGTTTCTGGGTGAGCAAAAACAGGAAGGCAAAATGCCGCAAAAAAGGGAATAA
GGGCGACACGGAAATGTTGAATACTCATACTCTTCCTTTTTCAATATTATTGAAGCATTTATCAGGGTTAT
TGTCTCATGAGCGGATACATATTTGAATGTATTTAGAAAAATAAACAAATAGGGGTTCCGCGCACATTTC
CCCGAAAAGTGCCACCTGACGTCGACGGATCGGGAGATCTCCCGATCCCCTATGGTGCACTCTCAGTAC
AATCTGCTCTGATGCCGCATAGTTAAGCCAGTATCTGCTCCCTGCTTGTGTGTTGGAGGTCGCTGAGTAG
TGCGCGAGCAAAATTTAAGCTACAACAAGGCAAGGCTTGACCGACAATTGCATGAAGAATCTGCTTAGG

### Example 2: Rapid and Highly Efficient Mammalian Cell Engineering Via Cas9 Protein Transfection

Abstract

CRISPR-Cas9 systems provide a platform for high efficiency genome editing that are enabling innovative applications of mammalian cell engineering. However, the delivery of Cas9 and synthesis of guide RNA (gRNA) remain as steps that can limit overall efficiency and general ease of use. Described here are methods for rapid synthesis of gRNA and for delivery of Cas9 protein/gRNA ribonucleoprotein complexes (Cas9 RNPs) into a variety of mammalian cells through liposome-mediated transfection or electroporation. Using these methods, nuclease-mediated indel rates of up to 94% in Jurkat T cells and 87% in induced pluripotent stem cells (iPSC) for a single target are reported. When this approach is used for multigene targeting in Jurkat cells, it was found that two-locus and three-locus indels were achieved in approximately 93% and 65% of the resulting isolated cell lines, respectively. Further, in this study, it was found that the off-target cleavage rate is significantly reduced using Cas9 protein when compared to plasmid DNA transfection. Taken together, a streamlined cell engineering workflow is presented that enables gRNA design to analysis of edited cells in as little as four days and results in highly efficient genome modulation in hard-to-transfect cells. The reagent preparation and delivery to cells requires no plasmid manipulation, and is thus amenable to high throughput, multiplexed genome-wide cell engineering.

Introduction

CRISPR-Cas9 mediated genome engineering enables researchers to modify genomic DNA in vivo directly and efficiently (Cho et al., “Targeted genome engineering in human cells with the Cas9 RNA-guided endonuclease,” Nat. Biotechnol. 31:230-232 (2013); Mali et al., “RNA-guided human genome engineering via Cas9,” Science 339:823-826 (2013); Jiang et al., “RNA-guided editing of bacterial genomes using CRISPR-Cas systems,” Nat. Biotechnol. 31:233-239 (2013); Wang et al., “One-step generation of mice carrying mutations in multiple genes by CRISPR/Cas-mediated genome engineering,” Cell 153:910-918 (2013)). Three components (Cas9, mature crRNA and tracrRNA) are essential for functional activity. Although the mature crRNA and tracrRNA can be synthesized chemically, the quality of the synthetic RNA is not sufficient for in vivo cell engineering due to the presence of truncated by-products (data not shown). Therefore, templates for the mature crRNA and tracrRNA or a combined single gRNA are often cloned into a Cas9 expression plasmid or built into separate plasmids driven by either U6 or H1 promoters for transcription after transfection of mammalian cells. Because the constructs are relatively large, delivery rates can be low, which would limit genomic cleavage efficiency, especially for hard-to-transfect cells. Recently, the use of Cas9 delivered as mRNA has led to increases in the rate of genomic cleavage in some cells. For example, a mixture of Cas9 mRNA and a single species of gRNA were co-injected into mouse embryonic stem (ES) cells resulting in biallelic mutations in 95% of newborn mice (Wang et al., “One-step generation of mice carrying mutations in multiple genes by CRISPR/Cas-mediated genome engineering,” Cell 153:910-918 (2013)). To make guide RNA, often precloned plasmid is used directly or a linear template is created via PCR amplification of the targeting sequence from a plasmid. If a 5′ T7 promoter does not appear in the plasmid, it is often added at this step and the resulting PCR product can be used in an in vitro transcription reaction. Alternatively, a synthetic DNA fragment containing a T7 promoter, crRNA and tracerRNA can be used as a template to prepare a gRNA by in vitro transcription. Overall, these represent a labor-intensive and time-consuming workflow, which led us to seek a simpler method to synthesize high quality gRNA. To that, describe here is a streamlined modular approach for gRNA production in vitro. Starting with two short single stranded oligos, the gRNA template is assembled in a ‘one pot’ PCR reaction. The product is then used as template in an in vitro transcription (IVT) reaction which is followed by a rapid purification step, yielding transfection-ready gRNA in as little as four hours.

To streamline the cell engineering workflow further, it was sought to eliminate any remaining cellular transcription or translation by directly introducing Cas9/gRNA ribonucleoprotein (RNP) complexes directly to the cells. Microinjection of Cas9 protein and gRNA complexes into C. elegans was first described in 2013 (Cho et al., “Heritable gene knockout in Caenorhabditis elegans by direct injection of Cas9-sgRNA ribonucleoproteins,” Genetics 195:1177-1180 (2013)) and was subsequently used to generate gene-knockout mice and zebrafish with mutation rates of up to 93% in newborn mice (Sung et al., “Highly efficient gene knockout in mice and zebrafish with RNA-guided endonucleases,” Genome Res. 24:125-131 (2014)). Following that report, Cas9 protein/gRNA complexes were delivered into cultured human fibroblasts and induced pluripotent stem cells (iPSC) via electroporation with high efficiency and relatively low off-target effects (Kim et al., “Highly efficient RNA-guided genome editing in human cells via delivery of purified Cas9 ribonucleoproteins” Genome Res. 24:1012-1019 (2014)). In that study, a large amount of Cas9 protein (4.5 to 45 μg) and gRNA (6 to 60 μg) were necessary for efficient genome modification (up to 79% indel efficiency). Most recently, delivery of Cas9 protein-associated gRNA complexes via liposomes was reported, in which RNAiMAX was used to deliver Cas9:sgRNA nuclease complexes into cultured human cells and into the mouse inner ear in vivo with up to 80% and 20% genome modification efficiency respectively (Zuris et al., “Cationic lipid-mediated delivery of proteins enables efficient protein-based genome editing in vitro and in vivo” Nat Biotechnol. October 30. doi: 10.1038/nbt.3081 (2014)).

The CRISPR/Cas system has been demonstrated as an efficient gene-targeting tool for multiplexed genome editing (Wang et al., “One-step generation of mice carrying mutations in multiple genes by CRISPR/Cas-mediated genome engineering,” Cell 153:910-918 (2013); Kabadi et al., “Multiplex CRISPR/Cas9-based genome engineering from a single lentiviral vector” Nucleic Acids Res. October 29; 42(19):e147. doi: 10.1093/nar/gku749 (2014); Sakuma et al., “Multiplex genome engineering in human cells using all-in-one CRISPR/Cas9 vector system,” Sci Rep. June 23; 4:5400. doi: 10.1038/srep05400 (2014); Cong et al., “Multiplex genome engineering using CRISPR/Cas systems. Science. 339: 819-823 (2013)). For example, co-transfections of mouse ES cells with constructs expressing Cas9 and three sgRNAs targeting Tet1, 2, and 3 resulted in 20% of cells having mutations in all six alleles of the three genes based on restriction fragment length polymorphism (RFLP) assay (Wang et al., “One-step generation of mice carrying mutations in multiple genes by CRISPR/Cas-mediated genome engineering,” Cell 153:910-918 (2013)). Lentiviral delivery of a single vector expressing Cas9 and four sgRNAs into primary human dermal fibroblasts resulted in about 30% simultaneous editing of four genomic loci among ten clonal populations based upon genomic cleavage detection assays (Kabadi et al., “Multiplex CRISPR/Cas9-based genome engineering from a single lentiviral vector” Nucleic Acids Res. October 29; 42(19):e147. doi: 10.1093/nar/gku749 (2014)). In one recent study, ‘all-in-one’ expression vectors containing seven guide RNA expression cassettes and a Cas9 nuclease/nickase expression cassette were delivered into 293T cells with genome cleavage efficiency ranging from 4 to 36% for each individual target (Sakuma et al., “Multiplex genome engineering in human cells using all-in-one CRISPR/Cas9 vector system,” Sci Rep. June 23; 4:5400. doi: 10.1038/srep05400 (2014)). In general, the efficiency of editing multiple genes in the human genome using plasmid-based delivery methods remains relatively low which subsequently increases the workload for downstream clonal isolation.

An in vitro gRNA production system has been developed and used a systematic approach to optimize the conditions for delivery of Cas9:gRNA complexes via lipid-mediated transfection or electroporation. A variety of mammalian cell lines were tested, including primary cells and other hard-to-transfect cells. Plasmid DNA, mRNA and Cas9 protein transfections were evaluated side by side. Using Cas9 protein transfection via electroporation, a superior genome editing efficiencies even in hard-to-transfect cells was achieved. In addition, the genome editing of multiple targets simultaneously using the Cas9 RNPs delivery system were assessed and are described here. It was found that delivery of Cas9 RNPs not only led to high indel production at single locus, but supports highly efficient biallelic modulation of at least two genes in a single transfection.

Materials and Methods

Materials:

293FT cells, The Gibco® Human Episomal iPSC Line, DMEM medium, RPMI 1640 medium, IMDM, DMEM/F-12, Fetal Bovine Serum (FBS), Knockout™ Serum Replacement, Non-Essential Amino Acid solution, basic fibroblast growth factor, Collagenase IV, TrypLE™ Express Enzyme, Geltrex, Opti-MEM Medium, FluoroBrite™ DMEM, Lipofectamine 2000, Lipofectamine 3000, RNAiMAX, Lipofectamine® MessengerMAX, GeneArt® CRISPR Nuclease Vector with OFP Reporter, 2% E-Gel® EX Agarose Gels, PureLink® PCR Micro Kit, TranscriptAid T7 High Yield Transcription Kit, MEGAclear™ Transcription Clean-Up Kit, Zero Blunt® TOPO® PCR Cloning Kit, PureLink® Pro Quick96 Plasmid Purification Kit, Endotoxin Quantitation Kit, Qubit® RNA BR Assay Kit, TRA-1-60 ALEXA FLUOR® 488 conjugated antibodies, SSEA4 ALEXA FLUOR® 647, and Phusion Flash High-Fidelity PCR Master Mix were from Thermo Fisher Scientific. Jurkat T cells and K562 cells were obtained from the American Type Culture Collection (ATCC). MEF feeder cells and ROCK inhibitor Y-27632 were purchased from EMD Millipore. Monoclonal Cas9 antibody was ordered from Diagenode. Recombinant Cas9 protein was purified as described by Kim et al. (7). All oligonucleotides used for gRNA synthesis were from Thermo Fisher Scientific (Supplementary Table 1s).

One Step Synthesis of gRNA Template

The 80 nt constant region of tracrRNA from a GeneArt® CRISPR Nuclease Vector was amplified by PCR and purified via agarose gel extraction. The concentration of PCR product was measured by Nanodrop (Thermo Fisher Scientific) and the molarity was calculated based on the molecular weight of 49.6 kDa. To prepare a pool of oligonucleotides, an aliquot of the 80 nt PCR product was mixed with two end primers and target-specific forward and reverse primers, with a final concentration of 0.15 μM for the 80 nt PCR product and 10 μM for each of the end primers. For a specific target, a 34 nt forward primer consisting of the T7 promoter sequence and 5′ end target sequence, and a 34 nt reverse primer consisting of the target sequence and 5′ end tracrRNA sequence were chemically synthesized with a 15 nt overlap. To set up the synthesis of gRNA template, aliquots of the pooled oligonucleotides were added to a Phusion Flash High-Fidelity PCR Master Mix and amplified using manufacturer's recommended reaction conditions. The PCR product was analyzed by a 2% E-Gel® EX Agarose Gel, followed by purification using Purelink PCR micro column. The gRNA template was eluted with 13 μl water and the concentration was determined by Nanodrop instrument.

To determine the error rate, the PCR product was cloned into Zero Blunt® TOPO® vector, followed by plasmid DNA isolation and sequencing with a 3500×1 DNA analyzer (Thermo Fisher Scientific).

In Vitro Transcription

The in vitro transcription of gRNA template was carried out using TranscriptAid T7 High Yield Transcription Kit using the manufacturer's recommended conditions. The gRNA product was purified using MEGAclear™ Transcription Clean-Up kit as described in the manual. The concentration of RNA was determined using Qubit® RNA BR Assay Kit.

Cell Culture

HEK 293FT cells were maintained in DMEM medium supplemented with 10% FBS. Jurkat T cells were propagated in RPMI medium containing 10% FBS, whereas K562 cells were cultured in IMDM medium supplemented with 10% FBS. Feeder-dependent human episomal iPSC were cultured on mitotically inactivated MEF feeder cells in human ESC (hESC) media containing 20% Knockout™ Serum Replacement, 10 μM Non-Essential Amino Acid solution, 55 μM 2-Mercaptoethanol, and 4 ng/ml basic fibroblast growth factor in DMEM/F-12. All cultures were maintained in a 5% CO₂, 37° C. humidified incubator. iPSC cultures were maintained with daily media changes and were passaged regularly using Collagenase IV.

Lipid-Mediated Cell Transfection

One day prior to transfection, the cells were seeded in a 24-well plate at a cell density of 2.5×10⁵ cells per well. For plasmid DNA transfection, 0.5 μg DNA was added to 25 μl of Opti-MEM medium, followed by addition of 25 μl of Opti-MEM containing 2 μl of Lipofectamine 2000. The mixture was incubated at room temperature for 15 minutes and then added to the cells. For Cas9 mRNA tranfection, 0.5 μg Cas9 mRNA (Thermo Fisher Scientific) was added to 25 μl of Opti-MEM, followed by addition of 50-100 ng gRNA. Meanwhile, 2 μl of Lipofectamine 3000 was diluted into 25 μl of Opti-MEM and then mixed with mRNA/gRNA sample. The mixture was incubated for 15 minutes prior to addition to the cells. For Cas9 protein transfection, 500 ng of purified Cas9 protein (Thermo Fisher Scientific) was added to 25 μl of Opti-MEM medium, followed by addition of 120 ng gRNA. The molar ratio of gRNA over Cas9 protein was approximately 1:1.2. The sample was mixed by gently tapping the tubes a few times and then incubated at room temperature for 10 minutes. To a separate test tube, 2 μl of RNAiMAX or Lipofectamine 3000 was added to 25 μl of Opti-MEM medium. The diluted transfection reagent was transferred to the tube containing Cas9 protein/gRNA complexes, followed by incubation at room temperature for 15 minutes. The entire solution was then added to the cells in a 24-well plate and mixed by gently swirling the plate. The plate was incubated at 37° C. for 48 hours in a 5% CO₂ incubator. The percentage of locus-specific indel formation was measured by GeneArt® Genomic Cleavage Detection Kit. The band intensities were quantitated using built-in software in Alpha Imager (Bio-Rad).

Electroporation

For suspension cells, such as Jurkat T cells or K562 cells, 2×10⁵ cells were used per electroporation using Neon® Transfection System 10 μL Kit (Thermo Fisher Scientific). To maximize the genome cleavage efficiency, the Neon 24 optimized protocol was applied according to the manufacturer's instruction. To set up a master mix, 24 μg of purified Cas9 protein was added to 240 μL of Resuspension Buffer R provided in the kit, followed by addition of 4.8 μg of gRNA. The mixture was incubated at room temperature for 10 minutes. Meanwhile, 4.8×10⁶ cells were transferred to a sterile test tube and centrifuged at 500×g for 5 minutes. The supernatant was aspirated and the cell pellet was resuspended in 1 ml of PBS without Ca²⁺ and Mg²⁺. Upon centrifugation, the supernatant was carefully aspirated so that almost all the PBS buffer was removed with no or minimum loss of cells. The Resuspension Buffer R containing the Cas9 protein/gRNA complexes was then used to resuspend the cell pellets. A 10 μl cell suspension was used for each of the 24 optimized conditions, which varied in pulse voltage, pulse width and the number of pulses. The electroporated cells were transferred immediately to a 24 well containing 0.5 ml of the corresponding growth medium and then incubated for 48 hours in a 5% CO₂ incubator. The cells were harvested by centrifugation and then washed once with PBS, followed by Genomic Cleavage and Detection assay as described by the manual. Upon optimization of electroporation condition, a higher amount of Cas9 protein (1.5 to 2 μg) and gRNA (300 to 400 ng) could be applied to further increase the genome editing efficiency. For each target in the multiplexing assays, 1 to 2 μg of Cas9 protein and 200-400 ng of gRNA were pre-incubated separately prior to mixing with cell pellet for electroporation. For clonal isolation, the cell number of transfected cells was counted upon 48 hour incubation, followed by a serial of dilution to 96 well plates with a cell density of 10-20 cells per plate based on the cell count. After clonal expansion for three weeks, cells from each individual well were harvested, followed by PCR amplification of the target locus. The PCR fragments were then cloned using a TOPO vector and transformed into TOP10 competent cells. Approximately 8 E. coli colonies were randomly picked for sequencing for each individual target locus. The single cell population was determined by the homogeneity of sequences for each allele. Single cells containing bi-allelic mutations on all desired targets were considered homozygotic indels. Downstream sequence analysis to confirm frame-shift induced stop codon introduction was not done.

For transfection of feeder free adaptation of iPSC, feeder dependent iPSC were grown to 80% confluency prior to harvest with collagenase. Following removal of the cell clusters from the feeder layer, they were gravity sedimented to prevent MEF contamination. The cell clusters were then seeded on to tissue culture dishes coated with Geltrex® in MEF conditioned media supplemented with 4 ng/mL bFGF. MEF conditioned media was produced using inactivated feeder cells, which was harvested on 7 continuous days, sterile filtered and frozen until usage. The cultures were allowed to reach 80-90% confluence. The day prior to transfection, the cultures were pretreated with 5 μM ROCK inhibitor Y-27632. On the day of harvest the cultures were inspected for signs of differentiation and any contamination differentiated cells were removed via micro-dissection. The cultures were washed once with DPBS and then harvested using TrypLE™ Express Enzyme. Single cells suspensions were counted using the Countess® automated cell counter. Following transfections, the cells were seeded onto multi-well (24 well) tissue culture dish coated with Geltrex® and incubated overnight with MEF conditioned media containing 5 μM ROCK. Media was replaced daily, without ROCK inhibitor, prior to analysis.

Cell Surface Immunostaining

To ensure maintenance of pluripotency post transfection and genome editing, iPSC cells were tested for expression of cell surface markers of self-renewal. The wells to be probed were washed with DMEM/F12 basal media. TRA-1-60 ALEXA FLUOR® 488 conjugated antibodies and SSEA4 ALEXA FLUOR®647 were multiplexed in basal DMEM/F-12 media. Both antibodies were added at a concentration of 2 μl of each antibody into 0.5 mL of pre-warmed DMEM/F-12 media and incubated at 37° C. for 45 minutes. Following the incubation, the antibody solution was removed and the wells were washed twice with DMEM/F-12. Prior to observation the media was exchanged with pre-warmed FluoroBrite™ DMEM. Images were taken using a Zeiss AxioVision microscope using a FITC and Cy5 laser/filter combination.

Analysis of Pluripotency Markers

Cultures were detached and dissociated using TrypLE™ Select and trituration. Single cell suspensions were incubated with TRA-1-60 ALEXA FLUOR® 488 conjugated antibodies and SSEA4 ALEXA FLUOR®647 for 1 hour at room temperature with gentle agitation. Two microliters (50×) of each antibody were added to 0.5 mL of DMEM/F-12. Following the incubation, the cells were centrifuged and washed once with Dulbecco's Phosphate-Buffered Saline (DPBS). After the removal of the DPBS wash, the pelleted cells were gently re-suspended in 1 mL of DPBS and stained through a strainer capped tube. The cells were then measured for the expression of both markers using the ATTUNE® Acoustic Focusing Cytometer and the data was analyzed using FlowJo® software.

Western Blot Analysis

293FT cells were transfected with either plasmid DNA, mRNA or Cas9 protein as described above. Cells were harvested at indicated times to perform both Genome Cleavage and Detection assay and Western Blot analysis. The cell lysate was fractionated using a 4-12% Novex Bis-tris gel. The proteins were transferred to a PVDF membrane using an IBLOT® following the manufacturer's protocol. Upon blocking, the membrane was incubated for 2 hours with monoclonal mouse Cas9 antibody at 1:3000 dilution. After washing, the membrane was incubated for 1 hour with rabbit anti-mouse antibody-HRP conjugate at 1:2000 dilution. Upon extensive washing, the membrane was developed with Pierce ECL reagent, followed by imaging using a Fuji imager LAS 4000 instrument.

Results

Three Day Cell Engineering Workflow

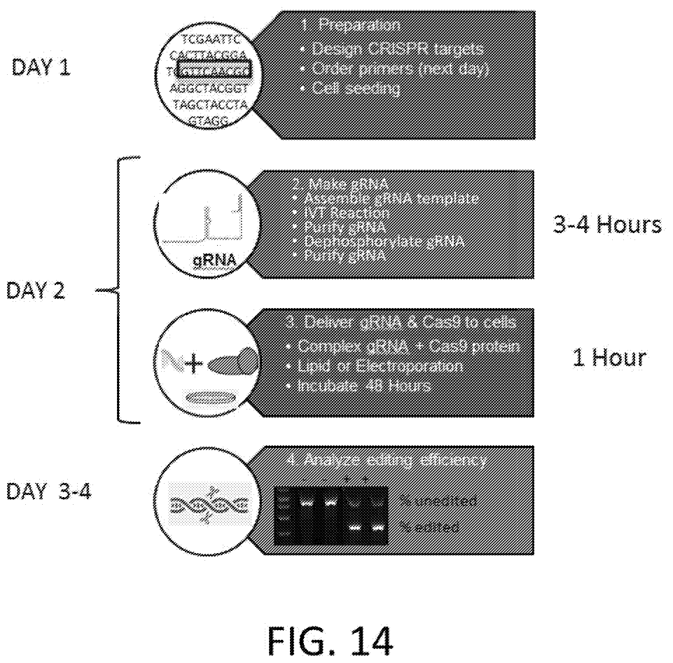

To streamline the genome engineering workflow, it was sought to simplify the gRNA synthesis procedure and shorten the time from experimental design to initial analysis as much as possible. Presented herein is a process where on day 1, the researcher designs and orders short DNA oligonucleotides and seeds the cells of interest for next day transfection (FIG. 14). Upon receiving the oligonucleotides on day 2, the researcher assembles the gRNA template in less than 1 hour by ‘one pot’ PCR. The resulting PCR product is then subjected to in vitro transcription to synthesize gRNA in approximately 3 hours. Upon association of gRNA with purified Cas9 protein, the Cas9 protein/gRNA complexes (Cas9 RNPs) are used to transfect cells via lipid-mediated delivery or electroporation. As early as day 3 (24 hours post transfection), the cells can be harvested for analysis of locus-specific genome modification efficiency.

To assemble the DNA template for gRNA production, a total of 4 synthetic DNA oligonucleotides and a purified PCR product representing the constant (non-targeting) crRNA region and tracrRNA sequence (gRNA lacking target sequence) are used. A pair of 34 nt forward and reverse oligonucleotide primers were designed by an online web tool (Beta Testing Version, Thermo Fisher Scientific), and share 15 nt homology with the CRISPR and tracer RNA regions respectively. The oligonucleotide pool concentrations as well as the PCR conditions were optimized such that the template was amplified in less than 40 minutes in a single tube with no obvious by-products. The gRNA template was used directly to prepare gRNA via in vitro transcription (IVT). The resulting gRNA was purified yielding high levels of gRNA with no detectable by-products. This approach was validated by synthesis of more than 96 distinct gRNAs. To determine the error rate in the synthetic DNA template, the PCR fragments were cloned and sequenced and it was found that approximately 7% of gRNA templates harbored mutations, mainly small deletions occurring at the extreme 3′ end and 5′ ends of the mature template. The use of HPLC-purified end primers further decreased the error rate to 3.6% with no mutations detected in the target region, which was similar to what was observed with the control template prepared from an ‘all-in-one’ plasmid with a 2% error rate. Taken together, this optimized process facilitates the conversion of a small set of DNA oligonucleotides into purified gRNA in approximately 4 hours with an accuracy of 96% and no errors detected in the targeting or Cas9 complexing (cr/tracrRNA) regions. Given that the process consists solely of liquid handling PCR, transcription, and RNA isolation steps, it is well suited for high throughput gRNA production and screening.

Liposome-Mediated Cas9 Protein Transfection

To examine the activity of synthetic gRNA, pre-complexed purified synthetic IVT gRNA with Cas9 protein were produced. It was hypothesizing that creating complexes of purified gRNAs with Cas9 protein prior to delivery to the cells might lead to higher genome editing efficiency due to the protection of the gRNA as it transits to the nucleus during the transfection process. To examine in vivo functionality of the system, human embryonic kidney (HEK293) cells were transfected with pre-complexed Cas9/gRNA ribonucleoproteins (Cas9 RNPs) using a set of cationic lipid reagents, followed by a genomic cleavage detection assay. Interestingly, the commonly-used plasmid DNA or RNA transfection reagents were able to efficiently deliver Cas9 RNPs. Lipofectamine 3000 and RNAiMAX outperformed Lipofectamine 2000 in HEK 293 cells (data not shown), which is in agreement with the recent finding that RNAiMAX performed better than Lipofectamine 2000 for delivery of Cas9 mRNA (Zuris et al., “Cationic lipid-mediated delivery of proteins enables efficient protein-based genome editing in vitro and in vivo” Nat Biotechnol. October 30. doi: 10.1038/nbt.3081 (2014)). For protein transfection, serum-free medium is generally used to avoid serum protein inference. In this study however, it was observed that the complete medium containing 10% FBS facilitated protein transfection and genome modification. The efficiencies of genome editing via plasmid DNA, mRNA and Cas9 RNP transfection were evaluated using three different target loci, HPRT, AAVS and RelA. Plasmid DNA and mRNA were delivered into HEK293 cells by Lipofectamine 3000, whereas Cas9 RNPs were delivered with RNAiMAX. The efficiencies of genome modification were similar among three target loci in DNA, mRNA and Cas9 protein-transfected cells.

Next examined was the kinetics of genome cleavage by transfecting cells with either plasmid DNA, mRNA or Cas9 RNPs, followed by genome cleavage assays and Western Blot analysis of cell lysates. In this study, it was observed similar cleavage kinetics between Cas9 delivered as plasmid DNA, mRNA and protein with efficient cleavage seen at 24 hours plateauing at 48 to 72 hours post-transfection in HEK293 cells. It was found that the kinetics of Cas9 RNP and mRNA encoded Cas9 appearance and turnover inside the transfected cells was quite different from that seen with Cas9 delivered via plasmid DNA. Measuring by Western Blot, it was found that Cas9 protein accumulated over time as expected in plasmid DNA-transfected cells, whereas the relatively low expression of Cas9 in mRNA-transfected cells seemed to peak as early as four hours post transfection and remained relatively stable for approximately 44 hours before diminishing. In the Cas9 RNP-transfected cells, the level of Cas9 protein peaked in four hours or less then rapidly decreased and was barely detectable in our assay at 48 hours. As a control, the blot membrane was stripped and re-probed with anti-actin antibody. Similar levels of actin expression were observed among samples (data not shown).

Because of the difference in protein appearance and apparent turnover rates, it was hypothesized that the off-target cleavage activity for Cas9 RNP transfection would be lower than that of plasmid DNA transfection. This was tested by targeting a locus in the VEGFA gene which has been identified as having several high activity off-target sites (Tsai et al., “GUIDE-seq enables genome-wide profiling of off-target cleavage by CRISPR-Cas nucleases,” Nat Biotechnol. doi:10.1038/nbt.3117 (2014)) via DNA, mRNA, and Cas9 RNP protein transfection followed by genome cleavage and locus sequencing analysis. Among the six potential off-target sites that have been studied previously (OT3-1, OT3-2, OT3-4, OT3-9, OT3-17 and OT3-18), only OT3-2 and OT3-18 were detected to harbor off-target mutation based on genome cleavage analysis. Further analysis of locus OT3-2 by sequencing indicated that the ratio of indel mutation of OT3-2 over on target in mRNA and Cas9 RNP transfected cells was 2 fold and 2.5 fold lower than that in DNA-transfected cells, respectively. The ratio of indel mutation of OT3-18 over on on-target was 1.6 fold and 28 fold lower in mRNA or Cas9 RNP-transfected cells respectively than in DNA-transfected cells. The on-target editing efficiency increased with an increased dose of Cas9 RNP, reaching plateau at around 2 μg of Cas9 protein, while the off-target modification at the loci examined remained low and constant (data not shown). Taken together, these data suggest that Cas9 delivery as mRNA and pre-complexed protein supports increased genomic cleavage specificity compared with standard DNA plasmid transfection.

Electroporation-Mediated Cas9 Protein Transfection

Many biologically and physiologically relevant cell lines, such as patient derived iPSC and progenitor cells, are refractory to efficient transfection by lipid-based reagents. Any improvement in the efficiency of genome modulation would facilitate isolation of appropriately engineered cells for experimentation and therapy so alternate means of delivering Cas9 RNPs and Cas9 mRNA/gRNA formulations and their effect on indel generation were explored. Using Jurkat T cells as an initial model, the delivery of Cas9 and gRNA plasmid DNA, Cas9 mRNA/gRNA formulations and Cas9 RNPs were compared using microporation (described in Materials and Methods, data not shown). Our results showed that, compared with plasmid DNA and mRNA deliveries, superior genome editing efficiency was achieved via delivery of Cas9 RNPs with approximately 90% HPRT locus-specific modification under several electroporation conditions. In general, Cas9 RNP delivery was more robust than DNA or mRNA delivery over most of the electroporation conditions tested. The cleavage efficiency was dose-dependent, reaching a maximum at approximately 1.5 μg Cas9 protein and 300 ng gRNA (˜1:1 molar ratio) per transfection. After sequencing the cell pools it was found that 94% of target loci harbored mutations at a cleavage site located at 3 bases upstream of NGG PAM sequence (Supplementary sequencing data). In agreement with previous work, the majority of mutations were distinct from each other with 73% insertion, 18% deletion and 3% base substitution. Given the high single-locus cleavage efficiency measured with the Cas9 RNP system, the ability to efficiently lesion multiple genes in a single transfection was tested. Here the capability of multiplexing Cas9 RNP transfection at three loci (AAVS1, RelA and HPRT) were examined. After pooling and delivering multiple species of Cas9 RNP (differing only by gRNA target), it was found that the efficiency of simultaneous editing of AAVS1/HPRT or AAVS1/RelA/HPRT loci was significantly greater at all loci compared with either plasmid or mRNA delivery of Cas9. To gain insight into the molecular level of multiplexing, one round of clonal isolation by serial dilution was performed. After clonal expansion each of the loci was PCR amplified, followed by DNA cloning and sequencing. In the case of two gene editing, it was found that all of 16 isolated clonal cell lines harbored bi-allelic indel mutations on single AAVS1 loci and 93.7% (15 of 16) of clonal cells harbored one allelic indel mutation at the HPRT locus as the HPRT target was located on the X chromosome of a male Jurkat T cell line. Overall, 93.7% of the clonal cell populations carried indel mutations on both the AAVS1 and HPRT loci. For multiplexing of three genes, three individual cell transfections and clonal isolation were performed with a total of 53 single cell lines analyzed. In this experiment, 90% and 65% of the clonal cell lines analyzed harbored bi-allelic indel mutations at the AAVS1 and RelA loci respectively, whereas 80% of the clonal cells carried indel mutations at the HPRT locus. Approximately 65% of the clonal cells carried bi-allelic indel mutations on both AAVS1 and RelA loci, whereas 80% and 65% of the clonal cells harbored indel mutations on AAVS1/HPRT loci and RelA/HPRT loci respectively. Overall, 65% of the clonal cell lines harbored indel mutations on all three targets. Further, 100% of the Jurkat T cell clones were edited at least once, suggesting that the transfection efficiency reached nearly 100%. Taken together, Cas9 RNP delivery via electroporation under the conditions used here achieved exceptionally high mutagenesis frequencies. This represents a substantial improvement in Cas9-mediated genome editing and significantly reduces the workload needed for clonal isolation by substantially reducing the number of cells that must be screened in order to identify and isolate the desired cell line.

Discussion

The ability to easily modulate the sequence specificity of the Cas9 nuclease by simply changing the 20 nucleotide targeting sequence of the gRNA offers significant versatility in delivery options over other nucleases that have been utilized for genome editing, such as zinc finger nucleases and TAL effectors. Now, researchers are able to choose from cost-effective and rapid design options by formulating the nuclease as either plasmid DNA, pre-made mRNA or purified protein. The design versatility is enabled by rapid production of the guide RNA component. Until recently, the gRNA was generally produced via cloning of a template sequence into a plasmid vector or vectors and expressing the Cas9 and gRNA in vivo. Described here is a streamlined protocol where gRNA design and template construction is facilitated by synthesis of two short single stranded oligonucleotides. The oligonucleotides are incorporated into gRNA templates via a short PCR reaction followed by conversion to gRNA by in vitro transcription. Target-specific oligos can be designed, ordered, and converted to purified gRNA in as little as two days. On the second day, the gRNA is formulated with either Cas9 mRNA or protein, and immediately used to transfect cells. The entire process consists completely of liquid handling and enzymatic reaction steps, which make it amenable to higher throughput gRNA production and transfection in multi-well plates.

The streamlined gRNA workflow was compared across the three delivery options and found that in general, Cas9/gRNA ribonucleoprotein complexes (Cas9 RNPs) offered superior indel production efficiency in most of the cell lines was used as a test bed. It is currently not clear why Cas9 RNP and total RNA formulations perform as they do but a factor could be overall size of the lipid complexes, the ability of Cas9 protein to protect the gRNA from cellular degradation, and the elimination of DNA-based cellular toxicity. In relation to plasmid delivery, Cas9 introduced as a Cas9 RNP or mRNA appears in the cell at low but evidently functional levels and is cleared rapidly which could also reduce the opportunity for off-target binding and cleavage. The data presented above suggests that this could be the case but a significantly more detailed evaluation is needed for confirmation.

Much progress has been made to reduce or eliminate off-target cleavage in CRISPR systems, such as use of paired Cas9 nickases and dimeric ‘dead Cas9’ FokI fusions, which has been shown to reduce off-target activity by 50- to 1,500-fold (Tsai et al., “GUIDE-seq enables genome-wide profiling of off-target cleavage by CRISPR-Cas nucleases,” Nat Biotechnol. doi:10.1038/nbt.3117 (2014); Fu et al., “High frequency off-target mutagenesis induced by CRISPR-Cas nucleases in human cells,” Nat. Biotechnol. 31:822-826 (2013)). Perhaps delivery of these tools via Cas9 RNPs would lead to even higher specificity while retaining high activity levels.

In this work, it was shown that it is possible to multiplex three Cas9 RNP species targeting separate loci in Jurkat T cells while achieving high levels indel production at all three loci. Further, it was observed high rates of biallelic modification at two diploid alleles (AAVS1 and RelA) in these experiments even when also modifying a haploid locus (HPRT) at similarly high levels. Taken together, the high rates of biallelic modification in cell populations suggest that employing Cas9 RNP delivery would significantly simplify the workflow by facilitating the selection of multigene knockout cell lines from a single experiment.

A survey was performed of eleven commonly used mammalian cell lines comparing CRISPR delivery via plasmid, Cas9 mRNA/gRNA, and Cas9 RNP (Table 10) and found that Cas9 mRNA/gRNA or Cas9 RNPs were superior to plasmid delivery in all cell lines tested. Delivery of these reagents via microporation offered the highest target-specific indel production under the conditions tested. In all but one case (NHEK cells), Cas9 RNP out performed Cas9 mRNA/gRNA and in human CD34+ cord blood cells, Cas9 RNP delivered via microporation was the only method that yielded a significantly robust editing solution.

TABLE 10
Transfection efficiency in variety of cell lines

| | DNA | | RNA | | Protein | |
|---|---|---|---|---|---|---|
| Cell lines | Lipid | Elect. | Lipid | Elect. | Lipid | Elect. |
| 293FT | 49.4 | 48.7 | 70 | 40.3 | 51.4 | 88 |
| U2OS | 15.0 | 50.3 | 21.4 | 23.6 ^{#} | 18.4 | 69.5 |
| Mouse ESCs | 30 | 45 | 45 | 20 | 25 | 70 |
| Human ESCs | 0 | 8 | 20 | 50 | 0 | 64 |
| (H9) | | | | | | |
| Human iPSCs | 0 | 20 | 66 | 31.6 | 0 | 87* |
| N2A | 65.8 | 75.7 | 65.6 | 80.2 | 66.3 | 82.3 |
| Jurkat | 0 | 63 | 0 | 42 | 0 | 94* |
| K562 | 0 | 45 | 0 | 27 | 0 | 72 |
| A549 | 15.0 | 44.3 | 23.1 | 28.7 | 19.7 | 65.5 |
| Human keratin. | 0 | 30 | 0 | 50 | 0 | 35 |
| (NHEK) | | | | | | |
| Human Cord | n/a | 5 | n/a | 0 | n/a | 24 |
| blood cells | | | | | | |
| CD34+ | | | | | | |

Notes:
HPRT for human cell lines and Rosa 26 for mouse cell lines
*confirmed by sequencing
^{#} Cleavage efficiency could be increased to 68% when Lipid was added into reaction before electroporation.

Described here is a streamlined approach to the mammalian genome engineering workflow that takes as few three days to modify mammalian genomes from CRISPR target design to evaluation of genome editing. To achieve a high mutagenesis efficiency in hard-to-transfect cells, a systematic approach was used to optimize transfection conditions and compare delivery of CRISPR editing tools via plasmid DNA, Cas9 mRNA/purified guide RNA (gRNA) formulations, and pre-complexed Cas9 protein and gRNA ribonucleoproteins (Cas9 RNPs). It was found Cas9 mRNA/gRNA and Cas9 RNP performance superior to ‘all-in-one’ plasmid DNA constructs in the variety of cell lines analyzed in this work. Most likely due to the high efficiency of Cas9 RNP delivery, it was possible to efficiently modify the genome at multiple loci simultaneously, thereby reducing the workload for downstream clonal isolation in schemes where more than one gene knock-out is desired. Further, it was found that delivery of Cas9 RNPs to cell lines considered hard to transfect (Jurkat, iPSC, CD4+) via electroporation yielded high levels of locus specific modification.

TABLE 11
Structure of Donor DNA Molecules

| Oligo | Sequence | SEQ ID |
|---|---|---|
| BT1/ | OOCTGGCCCACCCTCGTGACCACCTTCACCTFOG | |
| T8 | GEEACCGGGTGGGAGCACTGGTGGAAGTGGATEO | |
| 3OT1/ | OOCTGGCCCACCCTCGTGACCACCTTCACCTACGGCGZEC | |
| T8 | GEECACGGGACCGGGTGGGAGCACTGGTGGAAGTGGATEO | |
| 5OT1/ | OOCGTGCCCTGGCCCACCCTCGTGACCACCTTCACCTFOG | |
| T8 | GEEACCGGGTGGGAGCACTGGTGGAAGTGGATGCCGCAOE | |
| 3O/ | OZTCACCTACGGCGZEC | |
| T8 | CFOTGGTGGAAGTGGATEO | |
| 5O/ | EZGACCACCTTCACCTFOG | |
| T8 | GFFGTGGATGCCGCAOE | |
| BT2/ | OFCCGGCAAGCTGCCCGTGCCCTGGCCCACCCTCGTGACCACCTTCACCTFO | |
| T8 | G | |
| | GZEGCCGTTCGACGGGCACGGGACCGGGTGGGAGCACTGGTGGAAGTGGAT | |
| | EO | |
| 3OT/ | OFCCGGCAAGCTGCCCGTGCCCTGGCCCACCCTCGTGACCACCTTCACCTAC | |
| T8 | GGCGZEC | |
| | GFOGTGGTGGCCGTTCGACGGGCACGGGACCGGGTGGGAGCACTGGTGGAA | |
| | GTGGATEO | |
| 5OT2/ | OZGCACCACCGGCAAGCTGCCCGTGCCCTGGCCCACCCTCGTGACCACCTTC | |
| T8 | ACCTFOG | |
| | GZEGCCGTTCGACGGGCACGGGACCGGGTGGGAGCACTGGTGGAAGTGGAT | |
| | GCCGCAOE | |
| SS | P-OFTCTGCACCACCGGCAAGCTGCCCGTGCCCTGGCCCACCCTCGTGACCA | |
| | CCTTCACCTACGGCGTGCAGTGCTTCGCCCGCTACCCCGACCACFZG | |
| DS DNA | ATGGTGAGCAAGGGCGAGGAGCTGTTCACCGGGGTGGTGCCCATCCTGGTC | |
| | GAGCTGGACGGCGACGTAAACGGCCACAAGTTCAGCGTGTCCGGCGAGGG | |
| | CGAGGGCGATGCCACCTACGGCAAGCTGACCCTGAAGTTCATCTGCACCAC | |
| | CGGCAAGCTGCCCGTGCCCTGGCCCACCCTCGTGACCACCTTCACCTACGGC | |
| | GTGCAGTGCTTCGCCCGCTACCCCGACCACATGAAGCAGCACGACTTCTTCA | |
| | AGTCCGCCATGCCCGAAGGCTACGTCCAGGAGCGCACCATCTTCTTCAAGG | |
| | ACGACGGCAACTACAAGACCCGCGCCGAGGTGAAGTTCGAGGGCGACACC | |
| | CTGGTGAACCGCATCGAGCTGAAGGGCATCGACTTCAAGGAGG | |

Legend: F = Phosphorothioate-A, O = Phosphorothioate-C, E = Phosphorothioate-G, Z = Phosphorothioate-T
Regions of sequence homology are underlined

### Example 3: Preparation of Dried Reagents

Spray Drying:

A dry formulation of guide RNA is prepared from guide RNA, 1,2-Dipalmitoyl-sn-Glycero-3-Phosphocholine (DPPC), sucrose, and albumin (20:40:20:20 by weight). An aqueous solution containing 15 mg of siRNA, 15 mg of albumin, and 15 mg of sucrose (total volume 7.5 ml) is mixed with 17.5 ml of ethanol containing 30 mg of DPPC. Prior to combining the solutions they are mixed with a magnetic stir bar. After the aqueous solution is added to the organic solution, the combined solution was mixed by magnetic stir bar, at room temperature for about 6 minutes before the solution is spray dried. Conditions for spray drying are Tᵢₙₗₑₜ=95° C., Tₒᵤₜₗₑₜ=55° C., atomization/drying gas flow rate is 600 L/hr.

### Example 4

Background

For genome editing applications utilizing CRISPR/Cas9, it has been observed that the use of in vitro transcribed guide RNAs (gRNAs) can elicit toxic effects in a variety of cell types. This was possibly the result of immune activation through retinoic acid-inducible receptor I (RIG-I) activation, owing to the presence of triphosphate moieties found on the 5′ termini of gRNAs. Removal of these 5′ triphosphates with a phosphatase was tested to determine whether it would decrease gRNA cytotoxicity potentially by circumventing activation of RIG-I.

Methods

gRNA Synthesis and Dephosphorylation with CIAP

gRNAs were synthesized by in vitro transcription (IVT) using the GENEART™ Precision gRNA Synthesis Kit (Thermo Fisher Scientific, cat no. A29377) according to manufacturer's protocol. Following DNase I treatment, gRNAs were extracted with acid phenol:chloroform:isoamyl alcohol (PCI) and precipitated with NH₄OAc/isopropanol, washed twice with ice-cold 70% EtOH and re-suspended in RNase-free water. RNA concentrations were determined using the NanoDrop ND-1000 spectrophotometer (Thermo Scientific). Removal of 5′ triphosphates was carried out in reactions containing 20 μg gRNA, 1× Dephosphorylation buffer, and 40 U of Calf Intestinal Alkaline Phosphatase (CIAP, Thermo Fisher Scientific, cat. no. 18009019) in a total volume of 20 μL for 1 hour at 37° C. RNAs were then purified by PCI extraction and precipitation with NaOAc/EtOH and quantitated by NanoDrop spectrophotometry.

Transfection

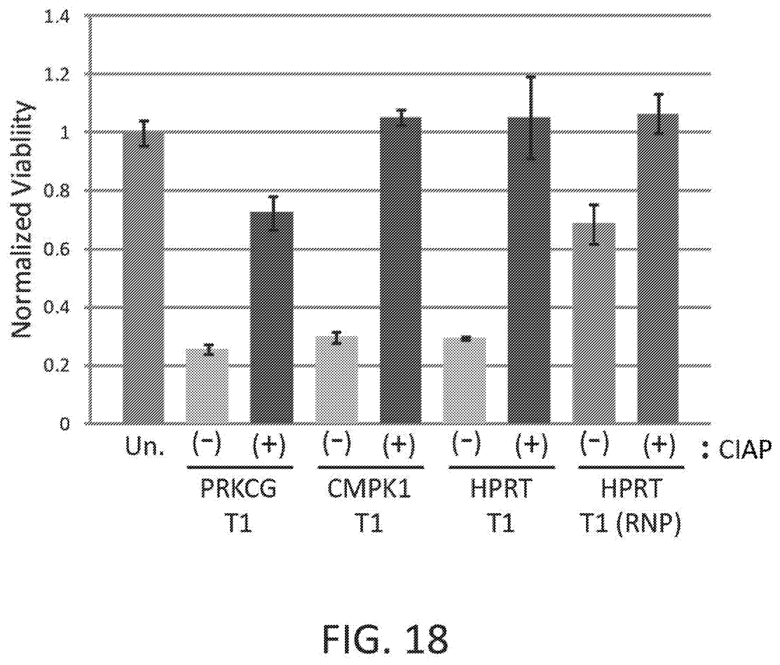

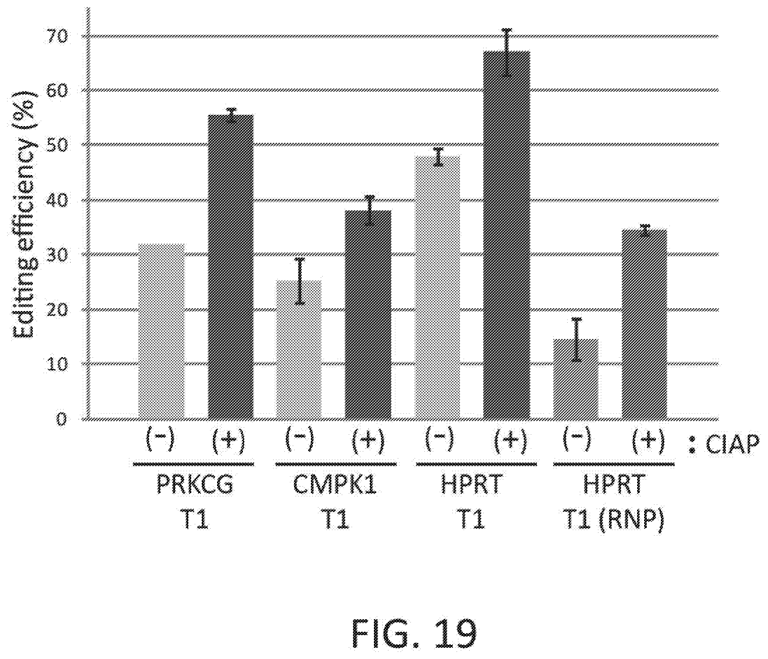

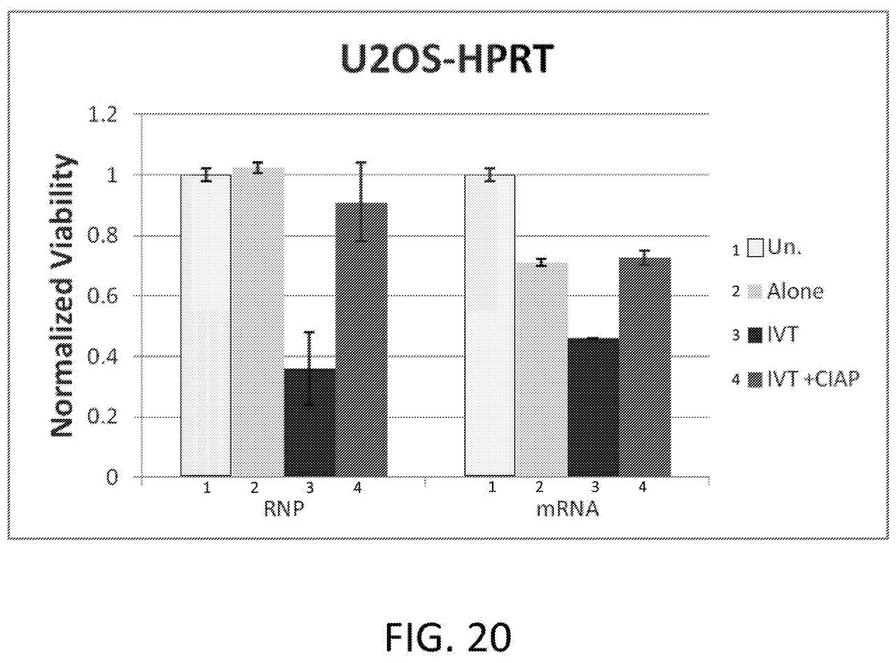

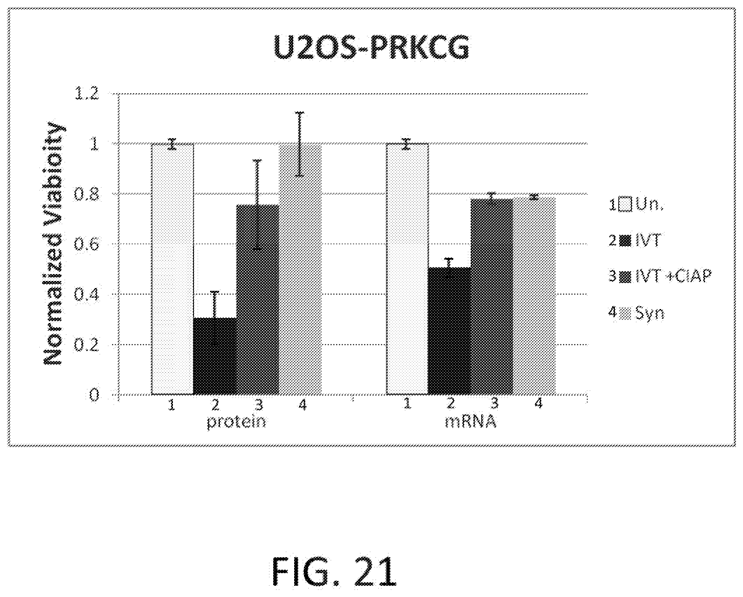

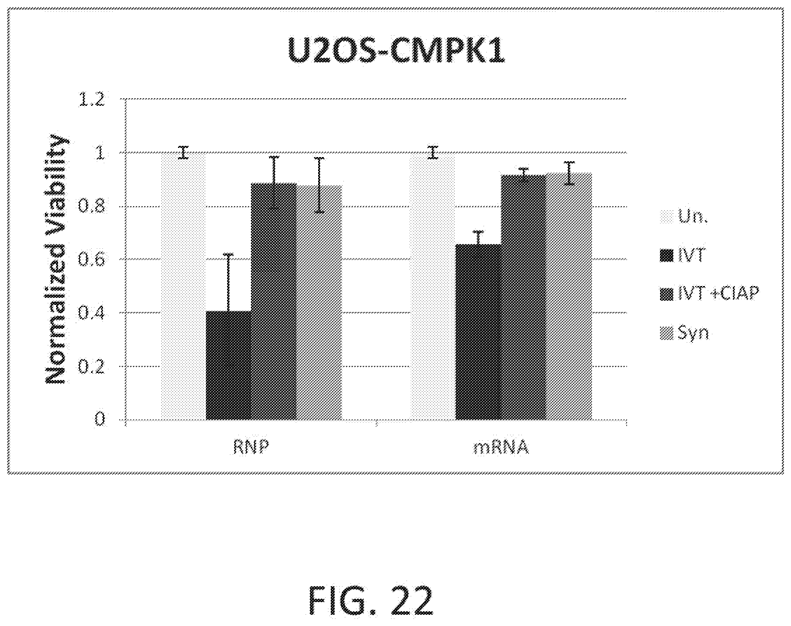

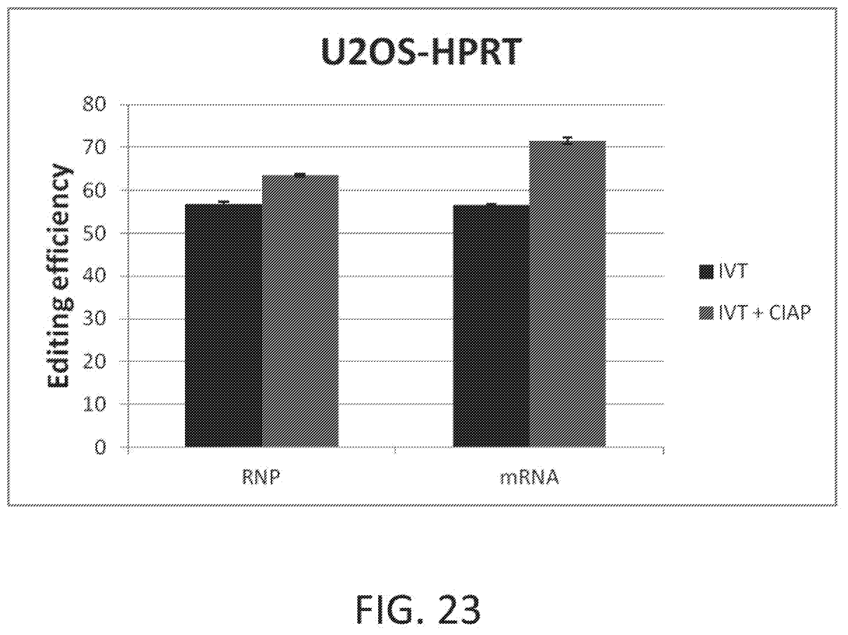

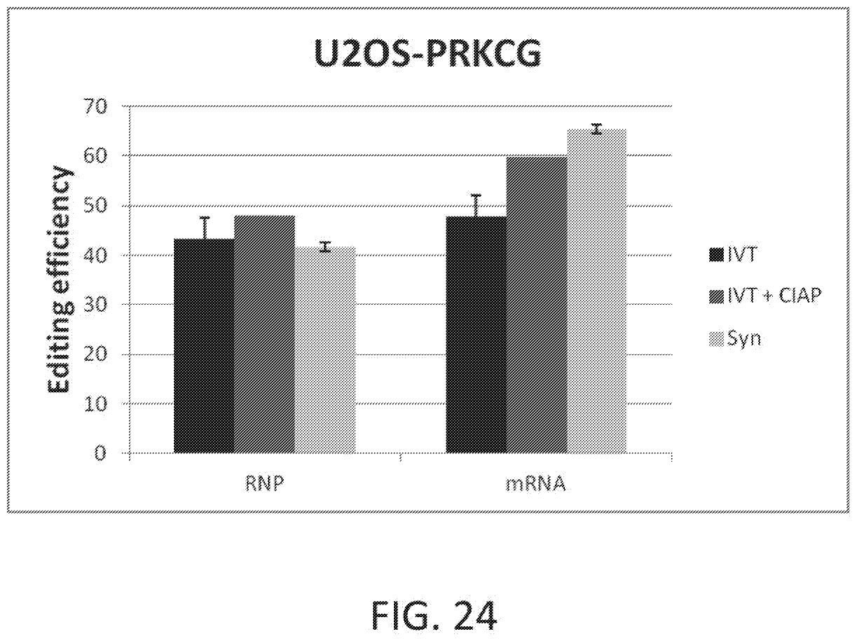

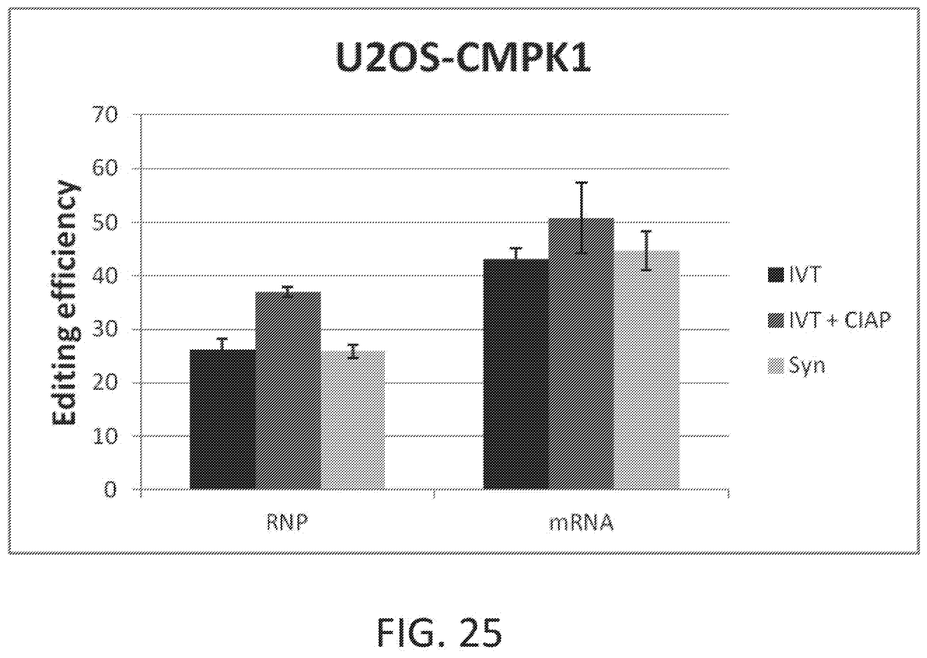

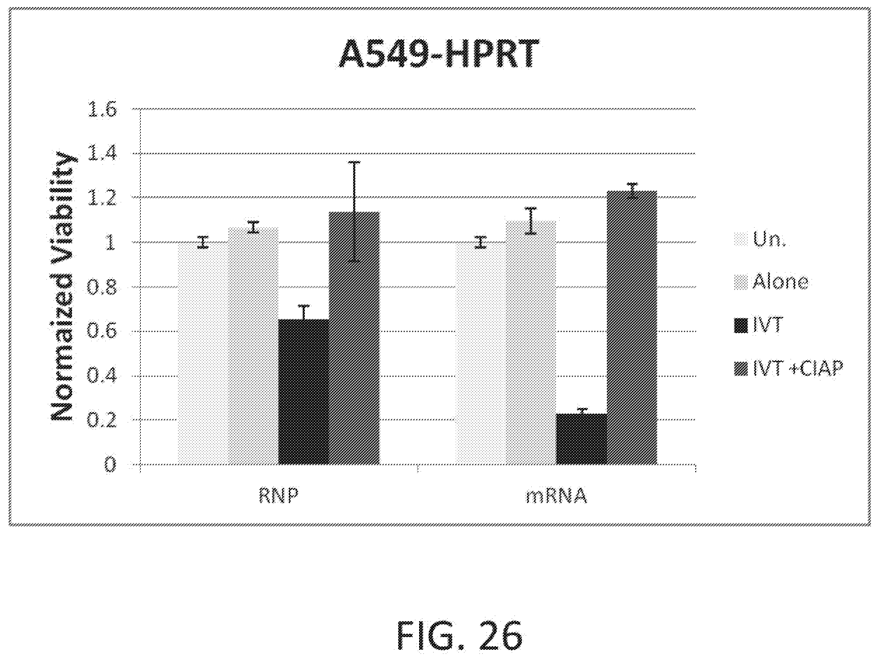

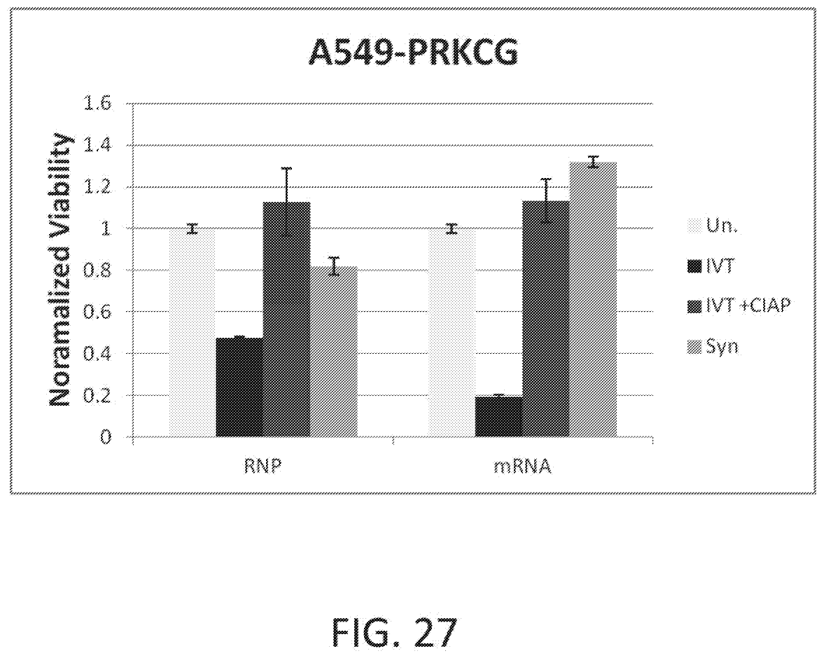

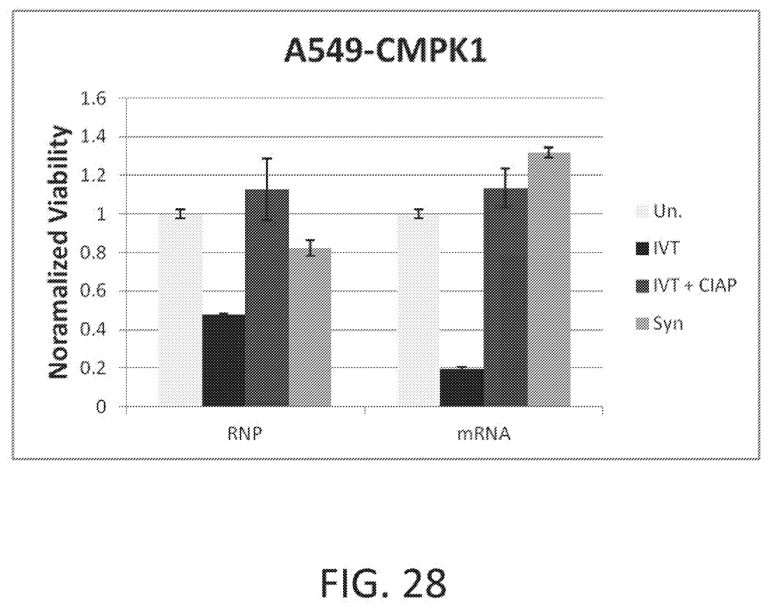

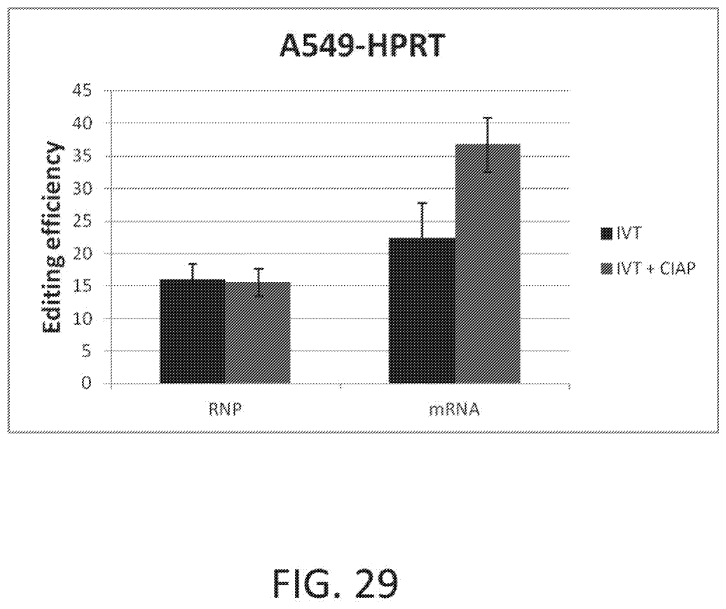

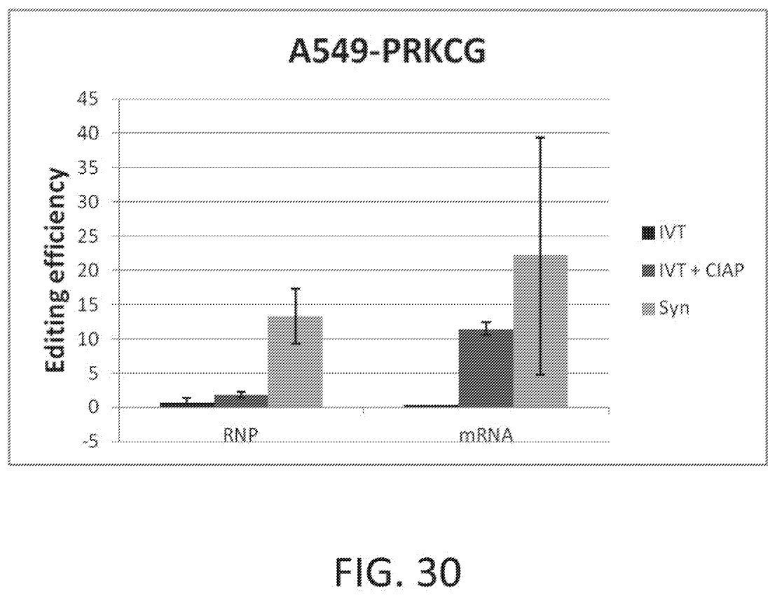

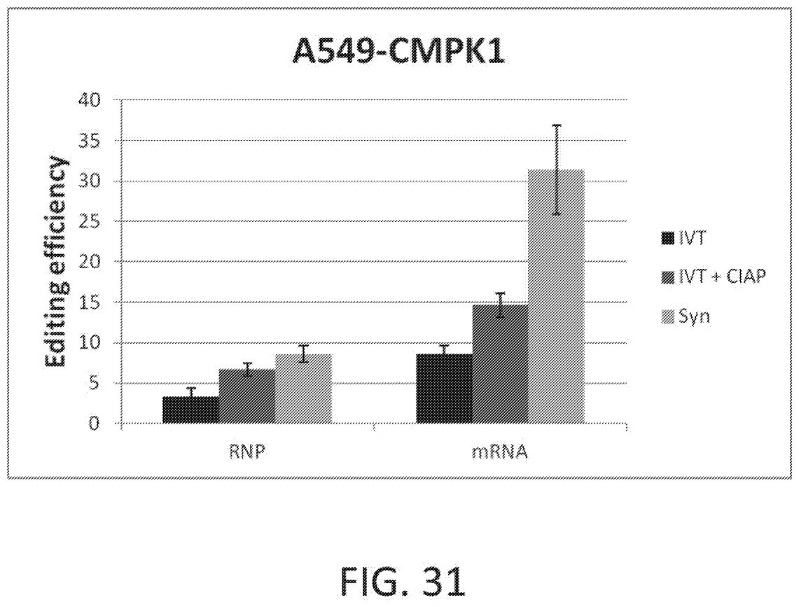

U2OS cells (10,000) in McCoy's 5 A+10% FBS (both Thermo Fisher Scientific) or A549 cells (16,000) in DMEM+10% FBS (Thermo Fisher Scientific) were seeded on 96 well plates. Twelve to fourteen hours later, the cells were transfected with 100 ng GENEART™ Platinum Cas9 Nuclease (Thermo Fisher Scientific, cat. no. B25640) or 125 ng of GENEART™ CRISPR nuclease mRNA (Thermo Fisher Scientific, cat. no. B29378) along with 20 ng of in vitro transcribed CIAP- or un-treated gRNAs targeting human HPRT1, PRKCG, or CMPK1 using LIPOFECTAMINE® RNAiMAX (Thermo Fisher Scientific, cat. no. 13778100) according to the manufacturers' recommendations. The in vitro transcribed gRNAs were compared to synthetic RNA oligo (IDT). Cell viability was assessed 48-72 hours later using PRESTOBLUE™ Cell Viability Reagent (Thermo Fisher Scientific, cat. no. A13261) according to manufacturers' recommendations. Cells were then harvested and genome editing was evaluated using GENEART® Genomic Cleavage Detection kit (GCD, Thermo Fisher Scientific, cat. no. 14372) according to the manufacturers' recommendations. gRNAs that had been “mock” CIAP-treated (incubated in the absence of CIAP) were tested to control for the effects of de-phosphorylation on cell viability and genome editing. Data obtained are set out in Tables 13 through 16 and FIGS. 18-31.

TABLE 12
Sequences of gRNA molecules and primers

HPRT T1
gRNA
Gcatttctcagtcctaaaca
gttttagagctagaaatagcaagttaaaataaggctagtccgttatcaac
ttgaaaaagtggcaccgagtcggtgctttt
HPRT GCD primers
Forward
ACATCAGCAGCTGTTCTG
Reverse
GGCTGAAAGGAGAGAACT
PRKCG
gRNA
ACTCGAAGGTCACAAATTCG
gttttagagctagaaatagcaagttaaaataaggctagtccgttatcaac
ttgaaaaagtggcaccgagtcggtgctttt
GCD primers
Forward
GACTCTGAGCCCATCTCTTGG
Reverse
CCATCTTCAGTCTCCTCACC
CMPK1
GTATTGAACGATGTCTTGAG
gttttagagctagaaatagcaagttaaaataaggctagtccgttatcaac
ttgaaaaagtggcaccgagtcggtgctttt
GCD primers
Forward
GTTTGAGGCATGTAATTCATACC
Reverse
GAGCTCACAGAACACTGGATCTG

TABLE 13
A549 (Normalized Viability)

| | | RNP | | mRNA | |
|---|---|---|---|---|---|
| | gRNA | Avg. | Stdev. | Avg. | Stdev. |
| HPRT | Un. | 1.000 | 0.020 | 1.000 | 0.020 |
| | Alone | 1.023 | 0.018 | 0.710 | 0.012 |
| | IVT | 0.358 | 0.120 | 0.458 | 0.003 |
| | IVT + CIAP | 0.911 | 0.129 | 0.727 | 0.023 |
| PRKCG | IVT | 0.307 | 0.104 | 0.506 | 0.036 |
| | IVT + CIAP | 0.757 | 0.179 | 0.782 | 0.022 |
| | Syn | 0.998 | 0.127 | 0.789 | 0.008 |
| | Mod Syn | 0.975 | 0.037 | 0.877 | 0.090 |
| CMPK1 | IVT | 0.408 | 0.210 | 0.658 | 0.046 |
| | IVT + CIAP | 0.888 | 0.097 | 0.918 | 0.024 |
| | Syn | 0.878 | 0.100 | 0.923 | 0.039 |
| | Mod Syn | 0.880 | 0.127 | 0.801 | 0.001 |

TABLE 14
U2OS (Normalized Viability)

| | | RNP | | mRNA | |
|---|---|---|---|---|---|
| | gRNA | Avg. | Stdev. | Avg. | Stdev. |
| HPRT | Un. | 1.000 | 0.022 | 1.000 | 0.022 |
| | Alone | 1.068 | 0.023 | 1.096 | 0.059 |
| | IVT | 0.652 | 0.062 | 0.227 | 0.022 |
| | IVT + CIAP | 1.139 | 0.223 | 1.230 | 0.031 |
| PRKCG | IVT | 0.475 | 0.006 | 0.192 | 0.012 |
| | IVT + CIAP | 1.127 | 0.160 | 1.134 | 0.102 |
| | Syn | 0.822 | 0.042 | 1.319 | 0.026 |
| | Mod Syn | 0.889 | 0.091 | 1.200 | 0.081 |
| CMPK1 | IVT | 0.787 | 0.148 | 0.175 | 0.002 |
| | IVT + CIAP | 1.121 | 0.107 | 1.158 | 0.110 |
| | Syn | 0.982 | 0.053 | 0.954 | 0.022 |
| | Mod Syn | 0.875 | 0.062 | 0.519 | 0.118 |

TABLE 15
A549 (% change in viability)

| | | RNP | | mRNA | |
|---|---|---|---|---|---|
| | gRNA | Avg. | Stdev. | Avg. | Stdev. |
| HPRT | Un. | 100.0% | 2.0% | 100.0% | 2.0% |
| | Alone | 2.3% | 1.8% | −29.0% | 1.2% |
| | IVT | −64.2% | 12.0% | −54.2% | 0.3% |
| | IVT + CIAP | −8.9% | 12.9% | −27.3% | 2.3% |
| PRKCG | IVT | −69.3% | 10.4% | −49.4% | 3.6% |
| | IVT + CIAP | −24.3% | 17.9% | −21.8% | 2.2% |
| | Syn | −0.2% | 12.7% | −21.1% | 0.8% |
| | Mod Syn | −2.5% | 3.7% | −12.3% | 9.0% |
| CMPK1 | IVT | −59.2% | 21.0% | −34.2% | 4.6% |
| | IVT + CIAP | −11.2% | 9.7% | −8.2% | 2.4% |
| | Syn | −12.2% | 10.0% | −7.7% | 3.9% |
| | Mod Syn | −12.0% | 12.7% | −19.9% | 0.1% |

TABLE 16
U2OS (% change in viability)

| | | RNP | | mRNA | |
|---|---|---|---|---|---|
| | gRNA | Avg. | Stdev. | Avg. | Stdev. |
| HPRT | Un. | 100.0% | 2.2% | 100.0% | 2.2% |
| | Alone | 6.8% | 2.3% | 9.6% | 5.9% |
| | IVT | −34.8% | 6.2% | −77.3% | 2.2% |
| | IVT + CIAP | 13.9% | 22.3% | 23.0% | 3.1% |
| PRKCG | IVT | −52.5% | 0.6% | −80.8% | 1.2% |
| | IVT + CIAP | 12.7% | 16.0% | 13.4% | 10.2% |
| | Syn | −17.8% | 4.2% | 31.9% | 2.6% |
| | Mod Syn | −11.1% | 9.1% | 20.0% | 8.1% |
| CMPK1 | IVT | −21.3% | 14.8% | −82.5% | 0.2% |
| | IVT + CIAP | 12.1% | 10.7% | 15.8% | 11.0% |
| | Syn | −1.8% | 5.3% | −4.6% | 2.2% |
| | Mod Syn | −12.5% | 6.2% | −48.1% | 11.8% |

While the foregoing embodiments have been described in some detail for purposes of clarity and understanding, it will be clear to one skilled in the art from a reading of this disclosure that various changes in form and detail can be made without departing from the true scope of the embodiments disclosed herein. For example, all the techniques, apparatuses, systems and methods described above can be used in various combinations.

The invention is further represented by the following clauses:

Clause 1. A method for introducing a dephosphorylated RNA molecule into a cell, the method comprising:
(a) performing in vitro transcription on a DNA molecule to form an RNA molecule,
(b) removing one or more terminal phosphate groups from the RNA molecule formed in (b) to produce a dephosphorylated RNA molecule, and
(c) contacting a cell with the dephosphorylated RNA molecule under conditions that allow for uptake of the dephosphorylated RNA molecule by the cell,
wherein the RNA molecule participates in gene editing or encode a protein that participates in gene editing.

Clause 2. The method of clause 1, wherein the RNA molecule is a guide RNA molecule or a messenger RNA (mRNA) molecule.

Clause 3. The method of clause 2, wherein the mRNA molecule encodes a protein selected from the group consisting of:
(a) a zinc finger protein,
(b) a TAL effector protein, and
(c) a Cas9 protein.

Clause 4. The method of clause 1, wherein the cell is an animal cell.

Clause 5. The method of any of clauses 1-4, wherein the animal cell is a human cell.

Clause 6. The method of any of clauses 1-4, wherein the animal cell is a mouse cell.

Clause 7. The method of any of clauses 1-4, wherein the dephosphorylated RNA molecule is contacted with the cell in the presence of a transfection reagent.

Clause 8. A method for producing a dephosphorylated RNA molecule, the method comprising:
(a) generating a DNA molecule by performing polymerase chain reactions (PCR) in a reaction mixture containing:
   (i) a double-stranded nucleic acid segment and
   (ii) at least one oligonucleotide capable of hybridizing to nucleic acid at one terminus of the double-stranded nucleic acid segment,
wherein the DNA molecule is produced by the PCR reaction, and
wherein the DNA molecule contains at or near one terminus a promoter suitable for in vitro transcription,
(b) performing in vitro transcription to form an RNA molecule, and
(c) removing one or more terminal phosphate groups from the RNA molecule formed in (b) to produce the dephosphorylated RNA molecule.

Clause 9. The method of clause 8, wherein the nucleic acid molecule produced by the PCR reaction encodes an RNA molecule from 35 to 150 nucleotides in length.

Clause 10. The method of clause 8, wherein the nucleic acid molecule produced by the PCR reaction is from 70 to 150 base pairs in length.

Clause 11. The method of any of clauses 8-10, wherein the nucleic acid molecule produced by the PCR reaction encodes an RNA molecule with at least two hairpin turns.

Clause 12. The method of any of clauses 8-11, wherein the nucleic acid molecule produced by the PCR reaction encodes a CRISPR RNA (crRNA).

Clause 13. The method of any of clauses 8-11, wherein the nucleic acid molecule produced by the PCR reaction encodes a guide RNA.

Clause 14. A method for producing a dephosphorylated RNA molecule, the method comprising:
(a) performing polymerase chain reaction (PCR) in a reaction mixture comprising:
   (i) a double-stranded nucleic acid segment comprising a first terminus and a second terminus,
   (ii) a first oligonucleotide comprising a first terminus and a second terminus, wherein the second terminus of the first oligonucleotide is capable of hybridizing to the first terminus of the double-stranded nucleic acid segment, and
   (iii) a second oligonucleotide comprising a first terminus and a second terminus, wherein the second terminus of the second oligonucleotide is capable of hybridizing to the first terminus of the first oligonucleotide, to produce the nucleic acid molecule,
wherein the product nucleic acid molecule contains a promoter suitable for in vitro transcription at or near one terminus and encodes a CRISPR RNA,
(b) performing in vitro transcription to form an RNA molecule, and
(c) removing one or more terminal phosphate groups from the RNA molecule formed in (b) to produce the dephosphorylated RNA molecule.

Clause 15. The method of clause 14, wherein the nucleic acid molecule is produced by the PCR reaction encodes a guide RNA.

Clause 16. The method of clause 14, wherein the reaction mixture further comprises a first primer and a second primer, wherein the first primer is capable of hybridizing at or near the first terminus of the second oligonucleotide and the second primer is capable of hybridizing at or near the second terminus of the double-stranded nucleic acid segment.

Clause 17. A method for producing a dephosphorylated RNA molecule, the method comprising:
(a) performing polymerase chain reaction in a reaction mixture containing
   (i) a first double-stranded nucleic acid segment comprising a first terminus and a second terminus,
   (ii) a second double-stranded nucleic acid segment comprising a first terminus and a second terminus, and
   (iii) at least one oligonucleotide comprising a first terminus and a second terminus,
wherein the first terminus of the oligonucleotide is capable of hybridizing to nucleic acid at the first terminus of the first double-stranded nucleic acid segment to produce the nucleic acid molecule,
wherein the second terminus of the oligonucleotide is capable of hybridizing to nucleic acid at the second terminus of the second double-stranded nucleic acid segment to produce the nucleic acid molecule, and
wherein the product nucleic acid molecule contains a promoter suitable for in vitro transcription at or near one terminus,
(b) performing in vitro transcription to form an RNA molecule, and
(c) removing one or more terminal phosphate groups from the RNA molecule formed in (b) to produce the dephosphorylated RNA molecule.

Clause 18. A method for producing a nucleic acid molecule, the method comprising:
(a) performing polymerase chain reaction in a reaction mixture containing:
   (i) a first double-stranded nucleic acid segment comprising a first terminus and a second terminus,
   (ii) a second double-stranded nucleic acid segment comprising a first terminus and a second terminus,
   (iii) a first oligonucleotide comprising a first terminus and a second terminus, and
   (iv) a second oligonucleotide comprising a first terminus and a second terminus,
wherein the second terminus of the first oligonucleotide is capable of hybridizing to nucleic acid at the first terminus of the second double-stranded nucleic acid segment, wherein the second terminus of the second oligonucleotide is capable of hybridizing to the first terminus of the first oligonucleotide,
wherein the second terminus of the second oligonucleotide is capable of hybridizing to the first terminus of the second double-stranded nucleic acid segment, and
wherein the product nucleic acid molecule contains a promoter suitable for in vitro transcription at or near one terminus,
(b) performing in vitro transcription to form an RNA molecule, and
(c) removing one or more terminal phosphate groups from the RNA molecule formed in (b) to produce the dephosphorylated RNA molecule.

Clause 19. A method for producing a CRISPR RNA molecule, the method comprising contacting two or more linear RNA segments with each other under conditions that allow for the 5′ terminus of a first RNA segment to be covalently linked with the 3′ terminus of a second RNA segment to form the CRISPR RNA molecules,
wherein terminal phosphate groups are removed from one or both termini of the CRISPR RNA.

Clause 20. The method of clause 19, wherein the CRISPR RNA molecule is separated from reaction mixture components.

Clause 21. The method of clause 20, wherein the CRISPR RNA molecules is separated from reaction mixture components by high-performance liquid chromatography.

Clause 22. A method for producing a guide RNA molecule, the method comprising:
(a) separately producing a crRNA molecule and a tracrRNA molecule, and
(b) contacting the crRNA molecule and the tracrRNA molecule with each other under conditions that allow for the covalently linking of the 3′ terminus of the crRNA to the 5′ terminus of the tracrRNA to produce the guide RNA molecule,
wherein terminal phosphate groups are removed from one or both termini of the guide RNA molecule.

Clause 23. The method of clause 22, wherein guide RNA molecule has a region of sequence complementarity of at least 10 nucleotides to a target locus.

Clause 24. The method of clause 23, wherein the target locus is a naturally occurring chromosomal locus in a eukaryotic cell.

Clause 25. A composition comprising two RNA molecules connected by a triazole group, wherein one of the RNA molecules has a region of sequence complementarity of at least 10 nucleotides to a target locus,
wherein terminal phosphate groups are removed from one or both termini of the two RNA molecules connected by a triazole group.

Clause 26. A method for gene editing at a target locus within a cell, the method comprising introducing into the cell at least one CRISPR protein and at least one CRISPR RNA molecule,
wherein the at least one CRISPR RNA molecule has a region of sequence complementarity of at least 10 base pairs to the target locus, and
wherein terminal phosphate groups are removed from one or both termini of the at least one CRISPR RNA molecule.

Clause 27. The method of clause 26, wherein a linear DNA segment that has sequence homology at both termini to the target locus is also introduced into the cell.

Clause 28. The method of any of clauses 26-27, wherein the at least one CRISPR protein is Cas9 protein.

Clause 29. The method of clause 28, wherein the Cas9 protein has the ability to make a double-stranded cut in DNA.

Clause 30. The method of clause 28, wherein two Cas9 proteins are introduced into the cell and each Cas9 protein has the ability to nick double-stranded DNA.

Clause 31. The method of clause 28, wherein one of the Cas9 proteins has a mutation that renders to HNH domain inactive and the other Cas9 protein has a mutation that renders to RuvC domain rendering that domain inactive.

Clause 32. The method of clause 28, wherein the Cas9 proteins have a mutation that renders either the HNH domain or the RuvC domain inactive.

Clause 33. The method of clause 26, wherein two RNA molecules, each with sequence complementarity to different target sequences, are introduced into the cell.

Clause 34. The method of clause 33, wherein the different target sequences are located within twenty base pairs of each other.

Clause 35. A composition comprising a mixture of capped mRNA molecules and uncapped mRNA molecules having a hydroxyl group on the 5′ terminus, wherein less than 3% of the mRNA molecules in the mixture contain a phosphate group at the 5′ terminus.

Clause 36. The composition of clause 35, wherein less than 1% of the mRNA molecules in the mixture contain a phosphate group at the 5′ terminus.

Clause 37. The composition of any of clauses 35-36, further comprising a transfection reagent.

Clause 38. The composition of any of clauses 35-37, wherein mRNA molecules in the mixture encode one or more of a Cas9 protein, a transcription activator-like effector protein, or a zinc finger protein.

Clause 39. The composition of clause 38, wherein the protein is a fusion protein.

Clause 40. The composition of clause 39, wherein the fusion protein contains one or more of a nuclear localization signal or a heterologous nuclease domain.

Clause 41. A method for preparing a population of mRNA molecules, the method comprising:
(a) performing in vitro transcription to generate a mixture containing capped mRNA molecules, and
(b) treating the mixture generated in (a) with a phosphatase under condition suitable for the removal of 5′ phosphate groups from mRNA molecules present in the mixture.

Clause 42. The method of clause 41, wherein the phosphatase is calf intestinal alkaline phosphatase.

1. A method for introducing a dephosphorylated RNA molecule into a cell, the method comprising:
(a) performing in vitro transcription on a DNA molecule to form an RNA molecule,
(b) removing one or more terminal phosphate groups from the RNA molecule formed in (b) to produce a dephosphorylated RNA molecule, and
(c) contacting a cell with the dephosphorylated RNA molecule under conditions that allow for uptake of the dephosphorylated RNA molecule by the cell,
wherein the RNA molecule participates in gene editing or encode a protein that participates in gene editing.

2. The method of claim 1, wherein the RNA molecule is a guide RNA molecule or a messenger RNA molecule.

3. The method of claim 2, wherein the mRNA molecule encodes a protein selected from the group consisting of:
(a) a zinc finger protein,
(b) a TAL effector protein, and
(c) a Cas9 protein.

4. The method of claim 1, wherein the cell is an animal cell.

5. The method of claim 4, wherein the animal cell is a human cell.

6. (canceled)

7. The method of claim 4, wherein the dephosphorylated RNA molecule is contacted with the cell in the presence of a transfection reagent.

8. A method for producing a dephosphorylated RNA molecule, the method comprising:
(a) generating a DNA molecule by performing polymerase chain reactions (PCR) in a reaction mixture containing:
(i) a double stranded nucleic acid segment and
(ii) at least one oligonucleotide capable of hybridizing to nucleic acid at one terminus of the double stranded nucleic acid segment,
wherein the DNA molecule is produced by the PCR reaction, and
wherein the DNA molecule contains at or near one terminus a promoter suitable for in vitro transcription,
(b) performing in vitro transcription to form an RNA molecule, and
(c) removing one or more terminal phosphate groups from the RNA molecule formed in (b) to produce the dephosphorylated RNA molecule.

9. The method of claim 8, wherein the nucleic acid molecule produced by the PCR reaction encodes an RNA molecule from 35 to 150 nucleotides in length.

10. The method of claim 8, wherein the nucleic acid molecule produced by the PCR reaction is from 70 to 150 base pairs in length.

11. The method of claim 8, wherein the nucleic acid molecule produced by the PCR reaction encodes an RNA molecule with at least two hairpin turns.

12. The method of claim 8, wherein the nucleic acid molecule produced by the PCR reaction encodes a CRISPR RNA (crRNA).

13. The method of claim 12, wherein the nucleic acid molecule produced by the PCR reaction encodes a guide RNA.

14.-25. (canceled)

26. A method for gene editing at a target locus within a cell, the method comprising introducing into the cell at least one CRISPR protein and at least one CRISPR RNA molecule,
wherein the at least one CRISPR RNA molecule has a region of sequence complementarity of at least 10 base pairs to the target locus, and
wherein terminal phosphate groups are removed from one or both termini of the at least one CRISPR RNA molecule.

27. The method of claim 26, wherein a linear DNA segment that has sequence homology at both termini to the target locus is also introduced into the cell.

28. The method of claim 26, wherein the at least one CRISPR protein is Cas9 protein.

29. The method of claim 28, wherein the Cas9 protein has the ability to make a double stranded cut in DNA.

30. The method of claim 28, wherein two Cas9 proteins are introduced into the cell and each Cas9 protein has the ability to nick double stranded DNA.

31. The method of claim 28, wherein one of the Cas9 proteins has a mutation that renders to HNH domain inactive and the other Cas9 protein has a mutation that renders to RuvC domain rendering that domain inactive.

32. (canceled)

33. The method of claim 26, wherein two RNA molecules, each with sequence complementarity to different target sequences, are introduced into the cell.

34. The method of claim 33, wherein the different target sequences are located within twenty base pairs of each other.

35.-42. (canceled)
